(19) United States
(12) Patent Application Publication
(10) Pub. No.: US 2009/0075281 A1
(43) Pub. Date: Mar. 19, 2009

(54) MTBE GENES

(75) Inventors: Krassimira R. HRISTOVA, Woodland, CA (US); Radomir Schmidt, Davis, CA (US); Anu Y. Chakicherla, Danville, CA (US); Kate M. Scow, Davis, CA (US); Staci R. Kane, Livermore, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: REGENTS OF THE UNIVERSITY OF CALIFORNIA, OAKLAND, CA (US)

(21) Appl. No.: 12/171,244

(22) Filed: Jul. 10, 2008

Related U.S. Application Data
(60) Provisional application No. 60/959,071, filed on Jul. 10, 2007.

(51) Int. Cl. C12Q 1/68; C07H 21/00; C12N 15/63; C12N 1/21; C07K 14/00

(52) U.S. Cl. 435/6; 435/262; 536/23.1; 435/320.1; 435/252.33; 530/350

(57) ABSTRACT

The present invention provides methods and compositions for modulating MTBE degradation. The invention also provides methods for identifying compounds that modulate MTBE expression.

### CROSS-REFERENCES TO RELATED APPLICATIONS

This application claims benefit of U.S. Provisional Patent Application No. 60/959,071, filed Jul. 10, 2007, the disclosure of which is incorporated by reference.

### STATEMENT AS TO RIGHTS TO INVENTIONS MADE UNDER FEDERALLY SPONSORED RESEARCH OR DEVELOPMENT

This work was supported by Grant No. 5 P42 ES04699-16 from the National Institute of Environmental Health Sciences (NIEHS), NIH. The government has certain rights in this invention.

### BACKGROUND OF THE INVENTION

Methylibium petroleiphilum strain PM1, a newly described genus and species (Nakatsu, C. H. et al., J Sys. Evol. Microbiol., 56:983-989 (2006)), is a motile bacterium belonging to the Comamonadaceae family of the beta-Proteobacteria and an important member of subsurface microbial communities in many gasoline-contaminated aquifers. Furthermore, PM1 is a methylotroph that can grow aerobically on the fuel oxygenate methyl tert-butyl ether (MTBE) and oxidize it completely to carbon dioxide (Bruns, M. A. et al., Environ. Microbiol., 3:220-225; Hanson, J. R. et al., Appl. Environ. Microbiol., 65:4788-4792 (1999)). MTBE is a suspected carcinogen that has contaminated drinking water wells throughout the US due to the preponderance of underground leaking storage tanks, the widespread usage of MTBE and its recalcitrance and mobility in groundwater. PM1 can also oxidize aromatic hydrocarbons (toluene, benzene, o-xylene, and phenol) (Deeb, R. A. et al., Environ. Sci. Technol., 35:312-317 (2001)) and n-alkanes (C₅-C₁₂) (Nakatsu, C. H. et al., J. Sys. Evol. Microbiol., 56:983-989 (2006); K. Hristova, unpublished data), and has been used in two bioaugmentation field trials in gasoline-contaminated aquifers in California (Smith, A. E. et al., Environ. Health Prospect., 113:317-332 (2005)) and Montana (Davis-Hoover, W. J. et al., BTEX/MTBE bioremediation: Bionets containing Isolite, PM1, SOS or air. E-25. In: V. S. Magar and M. E. Kelley (Eds.) Proceedings of the Seventh International In situ and On-site Bioremediation Symposium, Battelle Press, Columbus, Ohio (2003); Stavnes S. A. et al., MTBE bioremediation with BioNets containing Isolite, PM1, SOS or air. 2B-66. In: A. R. Gavaskar and A. S. C. Chen (eds.), Proceedings of the Third International Conference of Chlorinated and Recalcitrant Compounds, Battelle Press, Columbus, Ohio (2002)). In contaminated sites amended with oxygen, in situ MTBE degradation was observed and corresponded to increases in native populations of Methylibium sp. (˜99% similarity to PM1 based on 16S rDNA) (Hristova, K. et al., Appl. Environ. Microbiol., 69:2616-2623 (2003); Smith, A. E. et al., Environ. Health Prospect., 113:317-332 (2005); White, A. K. and W. W. Metcalf, J. Bacteriol., 186:4730-4739 (2004)). PM1-like bacteria occur naturally in a number of MTBE-contaminated aquifers in the US (Kane, S. R. et al., Appl. Environ. Microbiol., 67:5824-5829 (2001); Kane, S. R. et al., Aerobic biodegradation of MTBE by aquifer bacteria from LUFT sites. E-12. In: V. S. Magar and M. E. Kelley (Eds.) Proceedings of the Seventh International In Situ and On-site Bioremediation Symposium, Battelle Press, Columbus, Ohio (2003); Wilson, R. D. et al., Environ. Sci. Technol., 36:190-199 (2001)), Mexico (De Marco, P. et al., FEMS Lett., 234:75-80 (2004)) and Europe (Moreels, D. et al., FEMS Microbiol. Ecol., 49:121-128 (2004); Rohwerder, T. et al., Appl. Environ. Microbiol., 72:4128-4135 (2006)), and their presence has been correlated with MTBE degradation activity in numerous sites (Kane, S. R. et al., Aerobic biodegradation of MTBE by aquifer bacteria from LUFT sites. E-12. In: V. S. Magar and M. E. Kelley (Eds.) Proceedings of the Seventh International In Situ and On-site Bioremediation Symposium, Battelle Press, Columbus, Ohio (2003); Smith, A. E. et al., Environ. Health Prospect., 113:317-332 (2005); Wilson, R. D. et al., Environ. Sci. Technol., 36:190-199 (2001)) using real-time PCR analysis (Higgs, P. I. et al., J. Bacteriol., 180:6031-6038 (1998)). These results suggest that PM1-like organisms may play a major role in MTBE biodegradation under aerobic conditions in contaminated aquifers. The genetic basis for MTBE metabolism is not currently understood although there is general agreement that the initial enzymatic steps are similar to cometabolic degradation pathways (Fayolle, F. et al., Appl. Microbiol. Biotechnol., 56:339-349 (2001); Smith, C. A. et al., Appl. Environ. Microbiol., 69:796-804 (2003); Steffan, R. J. et al., Appl. Environ. Microbiol., 63:4216-4222 (1997)), and recent reports have described genes involved in degradation of MTBE downstream metabolites, 2-methyl-1,2-propanediol (Ferreira, N. L. et al., Microbiol., 152:1361-1374 (2006)) and 2-hydroxyisobutyrate (Rohwerder, T. et al., Appl. Environ. Microbiol., 72:4128-4135 (2006)). The complex regulation of the metabolism of fuel hydrocarbons and MTBE, often occurring in mixtures, is relatively unknown, while limited studies showed that MTBE degradation could be inhibited in mixtures with BTEX compounds (Deeb, R. A. et al., Environ. Sci. Technol., 35:312-317 (2001); Kane, S. R. et al., Aerobic biodegradation of MTBE by aquifer bacteria from LUFT sites. E-12. In: V. S. Magar and M. E. Kelley (Eds.) Proceedings of the Seventh International In Situ and On-site Bioremediation Symposium, Battelle Press, Columbus, Ohio (2003)).

Petroleum releases are among the most ubiquitous sources of composite organic contaminants in groundwater. The majority of petroleum-associated contaminants reach aquifers via spills or leaks from underground storage tanks (USTs) at service stations. Over 300,000 releases from USTs have been confirmed with more than 150,000 remediation efforts completed in the US (Llamas, M. A. et al., J. Bacteriol. 185:4707-4716 (2003)). Fuel oxygenates, such as methyl tertiary butyl ether (MTBE), often form extensive, unattenuated “plumes” in groundwater because of their high water solubility and low biodegradation rates under oxygen-limited conditions. MTBE was one of the major oxygenates incorporated into reformulated gasoline to increase the fuel's oxygen content and decrease carbon monoxide and ozone emissions. MTBE and its primary metabolite tert-butyl alcohol (TBA) are suspected and known carcinogens, respectively. Recently, alternative oxygenates such as ethanol are being substituted for MTBE, but because of the very slow depletion of contaminant mass from spill areas under anoxic conditions, the impacts of MTBE on the subsurface will be felt for many years and likely decades to come.

Methylibium petroleiphilum PM1 is one of the best-characterized aerobic MTBE-degraders known to date, and PM1-like bacteria have been shown to be present in several MTBE contaminated aquifers in California, (Hristova, K. R. et al., Appl. Environ. Microbiol. 69:2616-2623 (2003); Hristova, K. R. et al., Appl. Environ. Microbiol. 67:5154-5160 (2001)) (Kane, S. R. et al., Appl. Environ. Microbiol. 67:5824-5829 (2001)), and Europe (De Marco, P. et al., FEMS Microbiol. Lett. 234:75-80 (2004)) (Moreels, D. et al., Commun. Agric. Appl. Biol. Sci. 69:3-6 (2004)) (Rohwerder, T. et al., Appl. Environ. Microbiol. 72:4128-4135 (2006)). M. petroleiphilum PM1 uses MTBE as a sole carbon source, oxidizing it completely to CO₂ without accumulation of TBA (Hanson, J. R. et al., Appl. Environ. Microbiol. 65:4788-4792 (1999)). Strain PM1 has been used successfully in two bioaugmentation field trials in gasoline-contaminated aquifers in California (Smith, A. E. et al., Environ. Health Persp. 113:317-332 (2005)) and Montana (Davis-Hoover, W. J. et al., In V. S. M. a. M. E. Kelley (ed.), Seventh International In Situ and On-site Bioremediation Symposium. Battelle Press, Columbus, Ohio (2003)). M. petroleiphilum PM1 has a broad range of novel metabolic capabilities, including heterotrophic growth under aerobic conditions on diverse carbon sources (ethanol, methanol, toluene, benzene, ethybenzene, phenol, and C₄-C₁₂ n-alkanes (Deeb, R. A. et al., Environ. Sci. Technol. 35:312-317 (2001)) (Nakatsu, C. H. et al., Int. J. Sys. Evol. Microbiol. 56:983-989 (2006)) (Hristova, unpublished data). Impacts of interactions of MTBE and BTEX compounds (benzene, toluene, ethylbenzene, xylenes) on biodegradation capabilities of PM1 cultures have been demonstrated showing inhibition of MTBE degradation in the presence of certain BTEX compounds (Deeb, R. A. et al., Environ. Sci. Technol. 35:312-317 (2001); Kane, S. R. et al., In V. S. Magar and M. E. Kelley (ed.), Proceedings of the Seventh International In Situ and On-site Bioremediation Symposium. Battelle Press, Columbus, Ohio (2003)). However, the underlying biochemistry and complex regulation of the different pathways involved in biodegradation of these gasoline mixtures remains unknown.

To date, limited information is available about the genetics of MTBE biodegradation. A novel ether cleavage reaction has been described as the first step in MTBE oxidation for co-metabolic MTBE-degrading bacteria (Johnson, E. L. et al., Appl. Environ. Microbiol. 70:1023-1030 (2004); Smith, C. A. et al., Appl. Environ. Microbiol. 70:4544-4550 (2004); Smith, C. A. et al., Appl. Environ. Microbiol. 69:7385-7394 (2003); Steffan, R. J. et al. Appl. Environ. Microbiol. 63:4216-4222 (1997)); whether MTBE-metabolizing bacteria use a similar reaction is not yet known. Currently, there is no genetic information available concerning the identity and function of enzymes involved in MTBE and TBA oxidation in MTBE-metabolizing bacteria. However, recent studies elucidated the enzymes responsible for degradation of the MTBE metabolites, 2-methyl-2-hydroxy-1-propanol [or 2-methyl-1,2-propanediol] and hydroxyisobutyraldehyde in Mycobacterium austroafricanum IFP2012 (Ferreira, N. L. et al., Microbiol. 152:1361-1374 (2006)), and 2-hydroxyisobutyric acid (HIBA) in an environmental isolate phylogenetically similar to PM1 (Sanishvili, R. et al., J. Biol. Chem. 278:26039-26045 (2003)).

### BRIEF SUMMARY OF THE INVENTION

The present invention provides compositions and methods for modulating Methyl tertiary-butyl ether (MTBE) degradation and methods for identifying compounds that modulate MTBE degradation.

In one embodiment the methods for modulating MTBE degradation comprise modulating expression of a polypeptide selected from the group consisting of alkane 1-monooxygenase, dehydrogenase, tert-butyl alcohol hydroxylase, 2-methyl-2-hydroxy-1-propanol dehydrogenase, hydroxyisobutyraldehyde dehydrogenase, 2-hydroxy-isobutyryl-CoA ligase, 2-hydroxy-isobutyryl-CoA mutase, 3-hydroxy-butryl-CoA dehydrogenase, and combinations thereof. In some embodiments, the MTBE-mono-oxygenase, dehydrogenase is encoded by a nucleic acid comprising the sequence set forth in SEQ ID NO: 1; the tert-butyl alcohol hydroxylase is encoded by a nucleic acid comprising the sequence set forth in SEQ ID NO: 13 or 15; the 2-methyl-2-hydroxy-1-propanol dehydrogenase is encoded by a nucleic acid comprising the sequence set forth in SEQ ID NO: 17, hydroxyisobutyraldehyde dehydrogenase is encoded by a nucleic acid comprising the sequence set forth in SEQ ID NO: 19, the 2-hydroxy-isobutyryl-CoA mutase is encoded by a nucleic acid comprising the sequence set forth in SEQ ID NO:21 or 23, and the 3-hydroxy-butryl-CoA dehydrogenase is encoded by a nucleic acid comprising the sequence set forth in SEQ ID NO:25. In some embodiments, the MTBE-mono-oxygenase, dehydrogenase is encoded by a nucleic acid encoding the sequence set forth in SEQ ID NO: 2; the tert-butyl alcohol hydroxylase is encoded by a nucleic acid encoding the sequence set forth in SEQ ID NO: 14 or 16; the 2-methyl-2-hydroxy-1-propanol dehydrogenase is encoded by a nucleic acid encoding the sequence set forth in SEQ ID NO: 18, hydroxyisobutyraldehyde dehydrogenase is encoded by a nucleic acid encoding the sequence set forth in SEQ ID NO: 20, the 2-hydroxy-isobutyryl-CoA ligase is encoded by a nucleic acid encoding the sequence set forth in SEQ ID NO:29, the 2-hydroxy-isobutyryl-CoA mutase is encoded by a nucleic acid encoding the sequence set forth in SEQ ID NO:22 or 24, and the 3-hydroxy-butryl-CoA dehydrogenase is encoded by a nucleic acid encoding the sequence set forth in SEQ ID NO:26.

Another embodiment of the invention provides methods for identifying a compound that modulates MTBE degradation. The methods comprise (i) contacting a compound with a nucleic acid encoding a polypeptide selected from the group consisting of MTBE-mono-oxygenase, dehydrogenase, tert-butyl alcohol hydroxylase, 2-methyl-2-hydroxy-1-propanol dehydrogenase, hydroxyisobutyraldehyde dehydrogenase, 2-hydroxy-isobutyryl-CoA ligase, 2-hydroxy-isobutyryl-CoA mutase, 3-hydroxy-butryl-CoA dehydrogenase; and (ii) determining the effect of the compound upon the polypeptide, wherein a compound that increases or decreases the expression of the nucleic acid is identified as a compound that modulates MTBE degradation. the MTBE-mono-oxygenase, dehydrogenase is encoded by a nucleic acid comprising the sequence set forth in SEQ ID NO: 1; the tert-butyl alcohol hydroxylase is encoded by a nucleic acid comprising the sequence set forth in SEQ ID NO: 13 or 15; the 2-methyl-2-hydroxy-1-propanol dehydrogenase is encoded by a nucleic acid comprising the sequence set forth in SEQ ID NO: 17, hydroxyisobutyraldehyde dehydrogenase is encoded by a nucleic acid comprising the sequence set forth in SEQ ID NO: 19, the 2-hydroxy-isobutyryl-CoA mutase is encoded by a nucleic acid comprising the sequence set forth in SEQ ID NO:21 or 23, and the 3-hydroxy-butryl-CoA dehydrogenase is encoded by a nucleic acid comprising the sequence set forth in SEQ ID NO:25. In some embodiments, the MTBE-mono-oxygenase, dehydrogenase is encoded by a nucleic acid encoding the sequence set forth in SEQ ID NO: 2; the tert-butyl alcohol hydroxylase is encoded by a nucleic acid encoding the sequence set forth in SEQ ID NO: 14 or 16; the 2-methyl-2-hydroxy-1-propanol dehydrogenase is encoded by a nucleic acid encoding the sequence set forth in SEQ ID NO: 18, hydroxyisobutyraldehyde dehydrogenase is encoded by a nucleic acid encoding the sequence set forth in SEQ ID NO: 20, the 2-hydroxy-isobutyryl-CoA ligase is encoded by a nucleic acid encoding the sequence set forth in SEQ ID NO:29, the 2-hydroxy-isobutyryl-CoA mutase is encoded by a nucleic acid encoding the sequence set forth in SEQ ID NO:22 or 24, and the 3-hydroxy-butryl-CoA dehydrogenase is encoded by a nucleic acid encoding the sequence set forth in SEQ ID NO:26. In some embodiments, the effect is determined in vitro. In some embodiments, the nucleic acid is expressed in a host cell (e.g., E. coli). In some embodiments, the polypeptide is recombinant. In some embodiments, the compound is a small organic molecule

A further embodiment of the invention provides an isolated polynucleotide comprising the sequence set forth in SEQ ID NOS:1, 3, 5, 7, 9, 11, 13, 15, 17, 19, 21, 23, 25, or 27; expression vectors comprising the nucleic acid operably linked to an expression control sequence; and host cells (e.g., E. coli) comprising the expression vectors. Another embodiment of the invention provides an isolated polypeptide comprising an amino acid sequence encoded by a polynucleotide comprising the sequence set forth in SEQ ID NOS:1, 3, 5, 7, 9, 11, 13, 15, 17, 19, 21, 23, 25, or 27. Yet another embodiment of the invention provides an isolated polypeptide comprising the sequence set forth in SEQ ID NO: 2, 4, 6, 8, 10, 12, 14, 16, 18, 20, 22, 24, 26, 28, or 29.

In another aspect, the invention provides methods of detecting a polynucleotide of the invention, e.g., to detect the presence in a sample of interest of a bacteria that comprises the polynucleotide. The polynucleotide can be detected using any methodology known in the art. In some embodiments, the polynucleotide is detected using an amplification reaction, e.g., a polymerase chain reaction employing primer pairs that specifically target a polynucleotide of the invention, e.g., as set forth in SEQ ID NO:1, 3, 5, 7, 9, 11, 13, 15, 17, 19, 21, 23, 25, or 27.

### BRIEF DESCRIPTION OF THE DRAWINGS

FIG. 1 is Table 1 which sets forth general features of the Methylibium petroleiphilum PM1 genome.

FIG. 2 is Table 2 which sets forth M. petroleiphilum PM1 genes putatively involved in metabolism of methanol.

FIG. 3 is Table 3 which sets forth M. petroleiphilum PM1 genes putatively involved in metabolism of aromatic hydrocarbons denoting the predicted role of the gene product and percent similarity with well-characterized homologs.

FIG. 4 is Table 4 which sets forth genomic differences between the plasmid of PM1 with those from isolates MG4 and 312.

FIG. 5 is Supplemental Table 1 which sets forth M. petroleiphilum strain PM1 putative genes coding for proteins involved in cell motility, secretion, carbon fixation (non-functional), cobalamin biosynthesis, and proteins involved in energy transduction (TonB dependent)

FIG. 6 is Supplemental Table 2 which sets forth a summary of the genes found in M. petroleiphilum PM1 that are putatively involved in metal resistance, homeostasis and inorganic ion transport.

FIG. 7 is Supplemental Table 3 which sets forth putative monooxygenases, cytochromes and proteins involved in regulation and signaling in the genome of M. petroleiphilum PM1.

FIG. 8 is a table setting forth selected genes found in M. petroleiphilum PM1 involved in MTBE degradation.

### DETAILED DESCRIPTION OF THE INVENTION

The present invention is based on an analysis of the whole genome sequence of M. petroleiphilum PM1. We present comparative sequence analysis results between PM1 and bacteria with homologous individual genes and operons as well as comparative whole genomic hybridization analysis between PM1 and PM1-like MTBE-degrading isolates (˜99% identical 16S rDNA sequences) from gasoline-contaminated sites. General genome features are discussed including interesting repeated elements, as well as genes and operons involved in methylotrophy, degradation of aromatic hydrocarbons, degradation of cyclic and straight-chain alkanes, cofactor biosynthesis, motility, secretion, and heavy metal resistance and transport. A noteworthy finding was the presence of a large ˜600 bp plasmid in PM1 that was highly conserved among PM1-like bacteria. Furthermore, plasmid-curing experiments showed that the plasmid was essential for MTBE and TBA biodegradation in PM1. The PM1 genome sequence has provided a foundation for understanding novel pathways and interactions in this important subsurface bacterium as well as those in phylogenetically similar MTBE-degrading bacteria.

MTBE-mono-oxygenase, dehydrogenase; tert-butyl alcohol hydroxylase; 2-methyl-2-hydroxy-1-propanol dehydrogenase; hydroxyisobutyraldehyde dehydrogenase; 2-hydroxy-isobutyryl-CoA ligase; 2-hydroxy-isobutyryl-CoA mutase; or 3-hydroxy-butryl-CoA dehydrogenase refers to nucleic acids and polypeptide polymorphic variants (including single nucleotide polymorphisms involving displacement, insertion, or deletion of a single nucleotide that may or may not lead to a change in an encoded polypeptide sequence), alleles, mutants, and interspecies homologs that: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of over a region of at least about 25, 50, 100, 200, 500, 1000, or more amino acids, to an amino acid sequence encoded by a MTBE-mono-oxygenase, dehydrogenase; tert-butyl alcohol hydroxylase; 2-methyl-2-hydroxy-1-propanol dehydrogenase; hydroxyisobutyraldehyde dehydrogenase; 2-hydroxy-isobutyryl-CoA ligase; 2-hydroxy-isobutyryl-CoA mutase; or 3-hydroxy-butryl-CoA dehydrogenase nucleic acid (see, e.g., SEQ ID NOS: 1, 2, 3, 4, 5, 6, or 7, respectively); (2) bind to antibodies, e.g., polyclonal antibodies, raised against an immunogen comprising an amino acid sequence of a MTBE-mono-oxygenase, dehydrogenase; tert-butyl alcohol hydroxylase; 2-methyl-2-hydroxy-1-propanol dehydrogenase; hydroxyisobutyraldehyde dehydrogenase; 2-hydroxy-isobutyryl-CoA ligase; 2-hydroxy-isobutyryl-CoA mutase; or 3-hydroxy-butryl-CoA dehydrogenase polypeptide (e.g., encoded by SEQ ID NOS: 1, 3, 5, 7, 9, 11, 13, 15, 17, 19, 21, 23, 25, or 27), and conservatively modified variants thereof; (3) specifically hybridize under stringent hybridization conditions to an anti-sense strand corresponding to a nucleic acid sequence encoding a MTBE-mono-oxygenase, dehydrogenase; tert-butyl alcohol hydroxylase; 2-methyl-2-hydroxy-1-propanol dehydrogenase; hydroxyisobutyraldehyde dehydrogenase; 2-hydroxy-isobutyryl-CoA ligase; 2-hydroxy-isobutyryl-CoA mutase; or 3-hydroxy-butryl-CoA dehydrogenase protein, and conservatively modified variants thereof; (4) have a nucleic acid sequence that has greater than about 95%, preferably greater than about 96%, 97%, 98%, 99%, or higher nucleotide sequence identity, preferably over a region of at least about 25, 50, 100, 200, 500, 1000, or more nucleotides, to a MTBE-mono-oxygenase, dehydrogenase; tert-butyl alcohol hydroxylase; 2-methyl-2-hydroxy-1-propanol dehydrogenase; hydroxyisobutyraldehyde dehydrogenase; 2-hydroxy-isobutyryl-CoA ligase; 2-hydroxy-isobutyryl-CoA mutase; or 3-hydroxy-butryl-CoA dehydrogenase nucleic acid. Positions within the MTBE-mono-oxygenase, dehydrogenase; tert-butyl alcohol hydroxylase; 2-methyl-2-hydroxy-1-propanol dehydrogenase; hydroxyisobutyraldehyde dehydrogenase; 2-hydroxy-isobutyryl-CoA ligase; 2-hydroxy-isobutyryl-CoA mutase; or 3-hydroxy-butryl-CoA dehydrogenase protein nucleic acids are counted from nucleotide 1 of SEQ ID NO: 1, 3, 5, 7, 9, 11, 13, 15, 17, 19, 21, 23, 25, or 27, i.e., from the adenosine nucleotide of the ATG start codon. A polynucleotide or polypeptide sequence is typically from a mammal including, but not limited to, domesticated equines and wild equines. The nucleic acids and proteins of the invention include both naturally occurring or recombinant molecules.

The terms “nucleic acid” and “polynucleotide” are used interchangeably herein to refer to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A nucleic acid “capable of distinguishing” as used herein refers to a polynucleotide(s) that (1) specifically hybridizes under stringent hybridization conditions to an anti-sense strand corresponding to a nucleic acid sequence encoding a MTBE-mono-oxygenase, dehydrogenase; tert-butyl alcohol hydroxylase; 2-methyl-2-hydroxy-1-propanol dehydrogenase; hydroxyisobutyraldehyde dehydrogenase; 2-hydroxy-isobutyryl-CoA ligase; 2-hydroxy-isobutyryl-CoA mutase; or 3-hydroxy-butryl-CoA dehydrogenase protein, and conservatively modified variants thereof; or (2) has a nucleic acid sequence that has greater than about 80%, 85%, 90%, 95%, preferably greater than about 96%, 97%, 98%, 99%, or higher nucleotide sequence identity, preferably over a region of at least about 25, 50, 100, 200, 500, 1000, or more nucleotides, to a MTBE-mono-oxygenase, dehydrogenase; tert-butyl alcohol hydroxylase; 2-methyl-2-hydroxy-1-propanol dehydrogenase; hydroxyisobutyraldehyde dehydrogenase; 2-hydroxy-isobutyryl-CoA ligase; 2-hydroxy-isobutyryl-CoA mutase; or 3-hydroxy-butryl-CoA dehydrogenase nucleic acid (e.g., a sequence as set forth in SEQ ID NOS: 1, 3, 5, 7, 9, 11, 13, 15, 17, 19, 21, 23, 25, or 27, or complements or a subsequences thereof. MTBE-mono-oxygenase, dehydrogenase; tert-butyl alcohol hydroxylase; 2-methyl-2-hydroxy-1-propanol dehydrogenase; hydroxyisobutyraldehyde dehydrogenase; 2-hydroxy-isobutyryl-CoA ligase; 2-hydroxy-isobutyryl-CoA mutase; or 3-hydroxy-butryl-CoA dehydrogenase nucleic acids also include a sequence encoding SEQ ID NOS: 2, 4, 6, 8, 10, 12, 14, 16, 18, 20, 22, 24, 26, 28, or 29, or complements or a subsequences thereof.

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point I for the specific sequence at a defined ionic strength Ph. The Tₘ is the temperature (under defined ionic strength, Ph, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at Ph 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, optionally 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. In particular, an isolated MTBE-mono-oxygenase, dehydrogenase; tert-butyl alcohol hydroxylase; 2-methyl-2-hydroxy-1-propanol dehydrogenase; hydroxyisobutyraldehyde dehydrogenase; 2-hydroxy-isobutyryl-CoA ligase; 2-hydroxy-isobutyryl-CoA mutase; or 3-hydroxy-butryl-CoA dehydrogenase nucleic acid is separated from open reading frames that flank the MTBE-mono-oxygenase, dehydrogenase; tert-butyl alcohol hydroxylase; 2-methyl-2-hydroxy-1-propanol dehydrogenase; hydroxyisobutyraldehyde dehydrogenase; 2-hydroxy-isobutyryl-CoA ligase; 2-hydroxy-isobutyryl-CoA mutase; or 3-hydroxy-butryl-CoA dehydrogenase gene and encode proteins other than MTBE-mono-oxygenase, dehydrogenase; tert-butyl alcohol hydroxylase; 2-methyl-2-hydroxy-1-propanol dehydrogenase; hydroxyisobutyraldehyde dehydrogenase; 2-hydroxy-isobutyryl-CoA ligase; 2-hydroxy-isobutyryl-CoA mutase; or 3-hydroxy-butryl-CoA dehydrogenase. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, α-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine I, Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, preferably 65%, 70%, 75%, 80%, 85%, 90%, or 95% identity over a specified region of SEQ ID NO: 1, 3, 5, 7, 9, 11, 13, 15, 17, 19, 21, 23, 25, or 27, a polypeptide encoded by SEQ ID NO: 1, 3, 5, 7, 9, 11, 13, 15, 17, 19, 21, 23, 25, or 27), when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Preferably, the identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins to SREBP1, SCAP, INSIG1, INSIG2, MBTPS1, MBTPS2, or SCD5 nucleic acids and proteins, the BLAST and BLAST 2.0 algorithms and the default parameters discussed below are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat.'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (www.ncbi.nlm.nih.gov/). The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

By “host cell” is meant a cell that contains an expression vector and supports the replication or expression of the expression vector. Host cells may be, for example, prokaryotic cells such as E. coli or eukaryotic cells such as yeast or CHO cells.

### EXAMPLE 1

### Analysis of the Whole Genome Sequence of M. Petroleiphilum PM1

### Materials and Methods

Bacterial strains used in genome sequence and comparative hybridization analyses. Methylibium petroleiphilum strain PM1 was used for whole genome sequencing at the Joint Genome Institute (Walnut Creek, Calif.). Strain PM1 was isolated from a sewage treatment plant biofilter used for treating discharge from oil refineries (Hamamura, N. et al., Appl. Environ. Microbiol., 67:4992-4998 (2001); Bruns, M. A. et al., Environ. Microbiol., 3:220-225). Two MTBE-degrading bacterial pure cultures (MG4 and 312) were obtained from two different gasoline-contaminated aquifers in Northern California (Kane, S. R. et al., Appl. Environ. Microbiol., 67:5824-5829 (2001); Travis Air Force Base, Travis, Calif. and San Mateo, Calif., respectively). Enrichment culturing was conducted in 10 mg/L MTBE mineral salts media (MSM; Mu, D. Y. and K. M. Scow, Appl. Environ. Microbiol., 60:2661-2665 (1994)) with shaking at 150 rpm at room temperature. Enrichment cultures were plated onto 0.1× trypticase soy agar (TSA), and individual colonies were picked and grown in MSM with 10 mg/L MTBE and analyzed for MTBE degradation activity using purge-and-trap gas chromatography/mass spectrometry with reference to d₁₂-MTBE as an internal standard (Kane, S. R. et al., Appl. Environ. Microbiol., 67:5824-5829 (2001)). Culture purity was tested by plating (0.1×TSA) and 16S rDNA sequence analysis of colony genomic DNA.

Sequencing, gene prediction and annotation. Genomic DNA was isolated and purified from M. petroleiphilum PM1 and whole genome shotgun libraries (3-kb, 8-kb, and 40-kb DNA inserts) were constructed and sequenced as previously described (Chain, P. et al., J. Bacteriol., 185:2759-2773 (2003)). After quality control of the 90,327 total initial reads of draft sequence, 83,180 sequences were assembled, producing an average of 10.7-fold coverage across the genome. The whole genome sequence was assembled using the Phred/Phrap/Consed package (P. Green, University of Washington) (Ewing, B. and P. Green, Genome Res., 8:186-194 (1998a); Ewing, B. et al., Genome Res., 8:175-185 (1998b); Gordon, D. et al., Genome Res., 8:195-202 (1998)). The reads were assembled into 24 high-quality draft sequence contigs, which were linked into 3 larger scaffolds by paired-end sequence information. Gaps in the sequence were closed by either walking on gap-spanning clones or with PCR products generated from genomic DNA. Physical (un-captured) gaps were closed by combinatorial (multiplex) PCR. Sequence finishing and polishing added 308 reads, and the final assessment of the genome assembly was completed as described previously (Chain, P. et al., J. Bacteriol., 185:2759-2773 (2003)). The final genome assembly quality of PM1 adheres to conventional standards of less than one error per 10000 bp. Each base is covered by at least 2 quality sequences, with an average of 10.7 fold coverage. Proper assembly was verified by fosmid coverage coupled with PCR data. Gene modeling and genome annotation was performed as previously described (Chain, P. et al., J. Bacteriol., 185:2759-2773 (2003)) to identify open reading frames likely encoding proteins (coding sequences [CDS]).

Nucleotide sequence annotation and accession number. The annotation is available on the Joint Genome Institute web-site (http://genome.oml.gov/microbial/rgel/) and has been deposited in the GenBank/EMBL database under accession number NZ_AAEM00000000.

Comparative genomics. Orthologs and CDSs unique to M. petroleiphilum PM1 were identified using the Integrated Microbial Genomes (IMG) system from the Joint Genome Institute. Results were based on BLASTP analysis with cutoff values of E<10⁻⁵ and 30% identity for orthologs and E<10⁻² and 20% identity for unique CDSs.

Phylogenetic tree analysis. Homologs of M. petroleiphilum PM1 translated CDSs were identified using BLASTP searches against the non-redundant (NR) GenBank database from National Center for Biotechnology Information. Sequences were aligned and alignments were refined using ClustalX version 1.8 (Jeanmougin, F. et al., Trends Biochem. Sci., 23:403-405 (1998)) along with manual adjustments. The protdist program and the neighbor program of the Phylip package (Felsenstein, J., PHYLIP (Phylogeny Inference Package) version 3.6a3. Department of Genome Sciences, University of Washington, Seattle, Wash. (2002)) were used to generate the phylogenetic tree for MpeA3393. The pairwise parameters included gap opening=35 and gap extension=0.75. The multiple alignment parameters included gap opening=15, gap extension=0.3, delay divergence=30%, and Gonnet series for the protein weight matrix.

Comparative Genomic Hybridization and Comparative Genomic Sequencing analyses. Comparative Genomic Hybridization (CGH) was conducted in order to analyze conservation of genes from MTBE-degrading isolates MG4 and 312 with PM1 across the entire genome. High-density arrays (˜400,000 oligomers) were designed and produced by NimbleGen Systems, Inc. (Madison, Wis.) using 29-mer probes every 26 bp for both strands of the entire genome and every 7 bases for both strands of the plasmid. Arrays were hybridized with labeled genomic preparations of MG4, 312 and PM1. Genomic DNA was isolated (Ausubel, F. M. et al., Current protocols in molecular biology, Wiley, New York (1987)), and digested (5 μg per array) with 0.005 U DNase I (Amersham) in 1× One-Phor-All buffer (Amersham, Piscataway, N.J.) at 37° C. for 5 min with subsequent inactivation (95° C. for 15 min). To the DNA digest were added 4 μL 5× Terminal Transferase Buffer, 1 nmol Biotin-N6-ddATP, and 25 U Terminal Transferase. The sample was incubated at 37° C. for 90 min followed by inactivation at 95° C. for 15 min. Hybridization of arrays was conducted in 1× hybridization buffer for 16 hr at 45° C. using a Hybriwheel device (NimbleGen). PM1 was used as a reference in the analysis and was hybridized to separate arrays. Duplicate arrays were processed per strain. Arrays were washed with non-stringent wash buffer (6×SSPE, 0.01% [v/v] Tween-20) followed by two 5 min washes with stringent buffer (100 mM MES, 0.1 M NaCl, 0.01% [v/v] Tween-20) at 47.5° C. Arrays were stained with Cy3-streptavidin conjugate (Amersham Piscataway, N.J.) for 10 min followed by washing in nonstringent buffer. Signal amplification was achieved by secondary labeling with biotinylated goat anti-streptavidin (Vector Laboratories, Burlingame Calif.), washing in nonstringent buffer and restaining with Cy3-streptavidin. Finally, arrays were washed in non-stringent wash buffer, in 0.5×SSC two times for 30 sec and in 70% ethanol for 15 sec. Arrays were spun dry by centrifugation. Scanning was conducted at 5-┌m resolution with a Genepix 4000b scanner (Axon Instruments, Union City Calif.), and NimbleScan software (NimbleGen) was used to obtain pixel intensities. For higher resolution resequencing of the MG4 and 312 plasmids, arrays were synthesized and hybridized with genomic DNA from each strain and scanned as above. Single nucleotide polymorphism (SNP) positions were tested for uniqueness in the genome using custom algorithms (NimbleGen). The PM1 annotation (http://genome.ornl.gov/microbial/rgel/) was used to generate the output file in SignalMap analysis software (NimbleGen). The predicted SNPs were confirmed by producing and sequencing amplicons using PCR primers located external to the SNP location.

Random mutagenesis, mutant characterization and plasmid curing of PM1. In order to label the megaplasmid with a selectable marker, random transposon mutagenesis was employed using the mini transposon derivative, pTnMod-SmO containing the streptomycin/spectinomycin adenylyltransferase gene (aadA) and an oriR origin of replication between the inverted repeats (Dennis, J. J and J. G. Zylstra, Appl. Environ. Microbiol., 64:2710-2715 (1998)). Electrocompetent PM1 cells were prepared by culturing in 0.5× Tryptic Soy Broth (TSB) at 27° C. with shaking to log phase. Cells were collected by centrifugation, washed in 10% glycerol four times, and suspended in 10% glycerol to a final volume of 100 ┌1. A mixture of 50 ┌1 cells and 2 ┌1 pTnMod-SmO DNA (1 μg/μl) was electroporated in 0.1 mm gap cuvettes using 1.8 kV, 200 Ohms, and 25 ┌F capacitance settings (Dennis, J. J and J. G. Zylstra, Appl. Environ. Microbiol., 64:2710-2715 (1998)) in a BioRad Gene Pulser Electroporator (BioRad, Hercules, Calif.). Following a 4 h recovery in 0.5×TSB at 27° C. with shaking, transposon mutants were selected on 0.5×TSA plates with 50 ┌g/ml streptomycin (Sm). Sm-resistant colonies were present after incubation for 5-6 days at 27° C. and stable transposon integration was confirmed by PCR analysis of genomic DNA using pTnMod-SmO specific primers.

Using the rapid cloning strategy outlined by Dennis et al. (Dennis, J. J and J. G. Zylstra, Appl. Environ. Microbiol., 64:2710-2715 (1998)), the <SmO> insert location was mapped in several PM1 subclones containing the oriR within the transposon. Briefly genomic DNA was extracted, digested with Ava II restriction endonuclease, self-ligated, and transformed into E. coli TOP 10 cells (Invitrogen, Carlsbad, Calif.). The resulting transformants were selected on LB agar containing 50 ┌g/ml Sm. Sequencing with primers against the ends of the <SmO> insert was used to determine the exact insert location. One transposon-mutant MP0005 was shown to have the <SmO> insert on the megaplasmid (in MpeB636). MP0005 was subjected to plasmid curing by heat stress as described by Trevors (Trevors, J. T., FEMS Microbiol. Rev., 32:149-157 (1986)). Specifically, strain MP0005 was incubated at 37° C. for 6-8 h before plating on 0.5×TSA. Following replica plating on 0.5×TSA with and without 50 ┌g/ml Sm, Sm-sensitive colonies were selected and megaplasmid loss was confirmed by PCR analysis. MTBE and TBA degradation activity by strain MP0005 and a megaplasmid-free strain MP0007 were determined by gas chromatography analysis as previously described (Hanson, J. R. et al., Appl. Environ. Microbiol., 65:4788-4792 (1999)).

### Results and Discussion

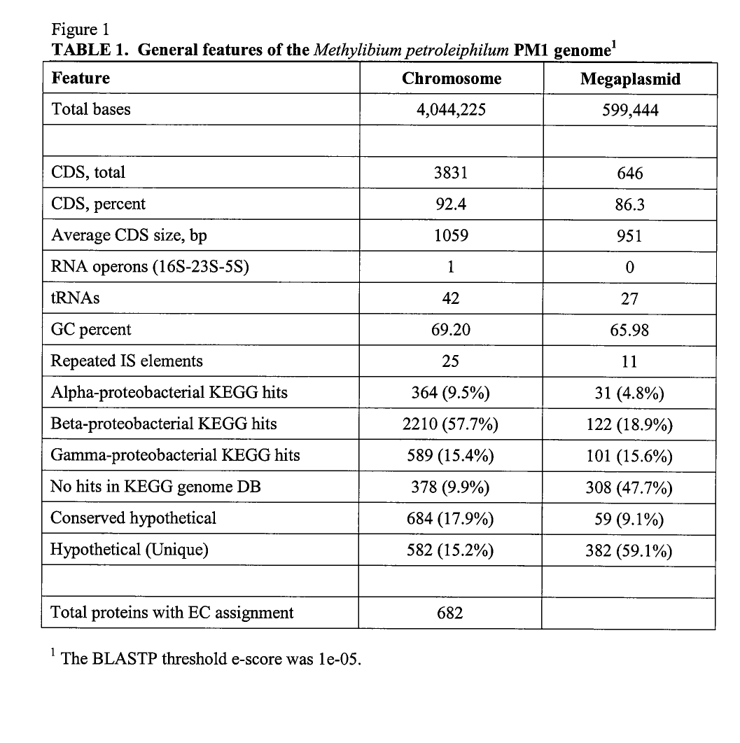

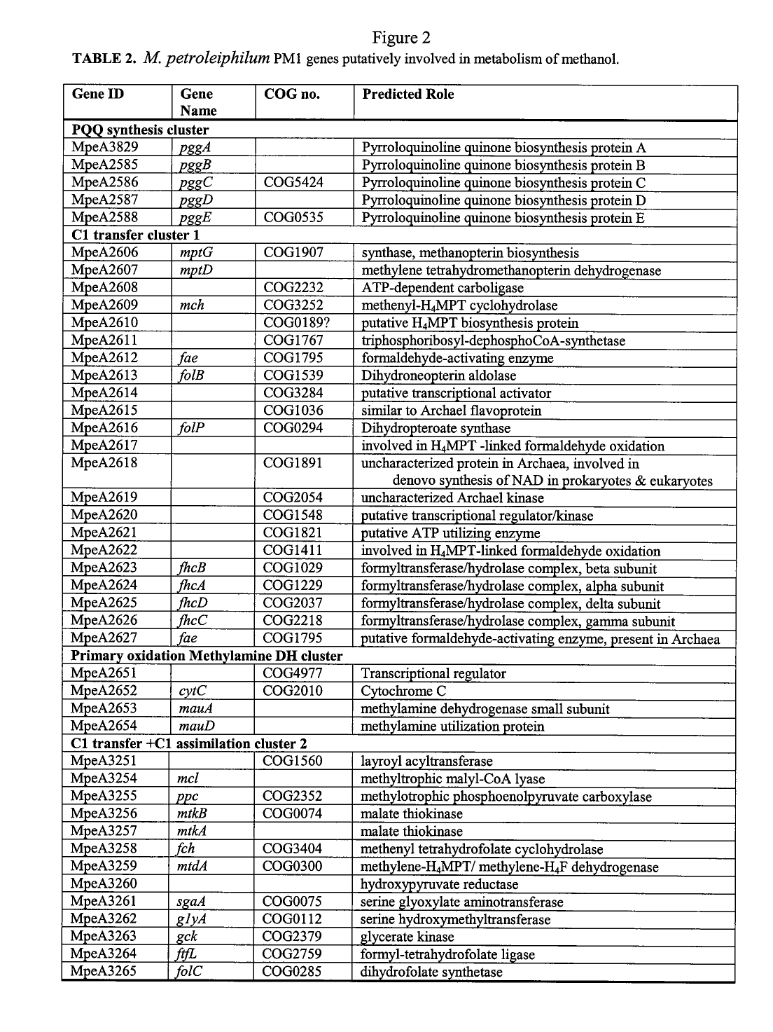

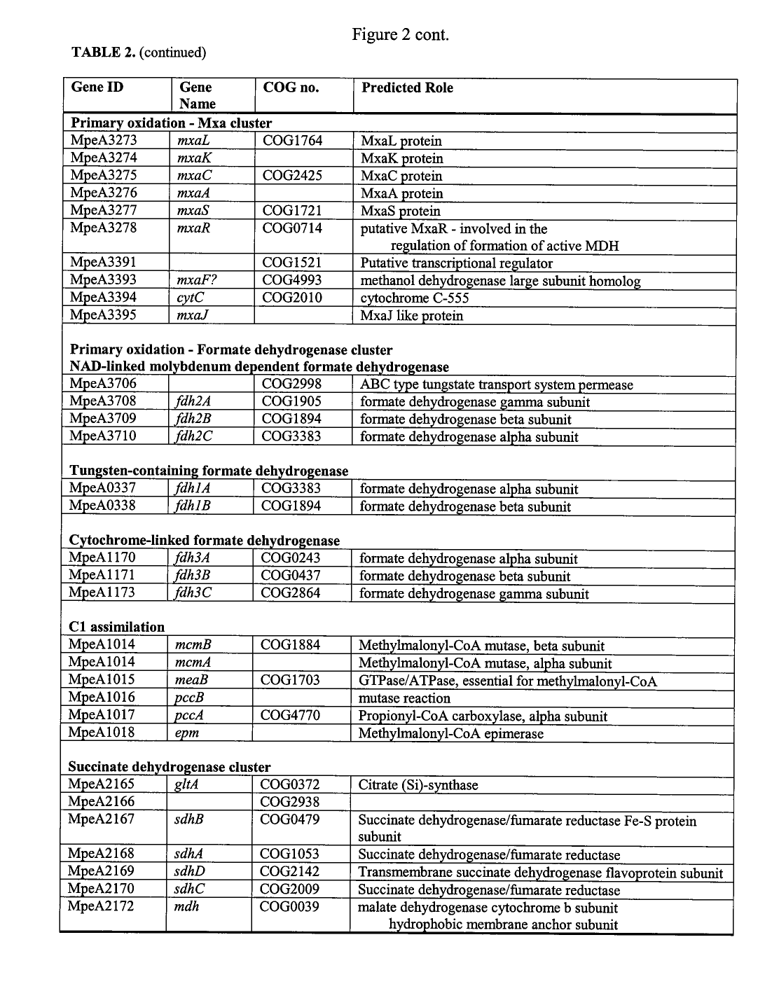

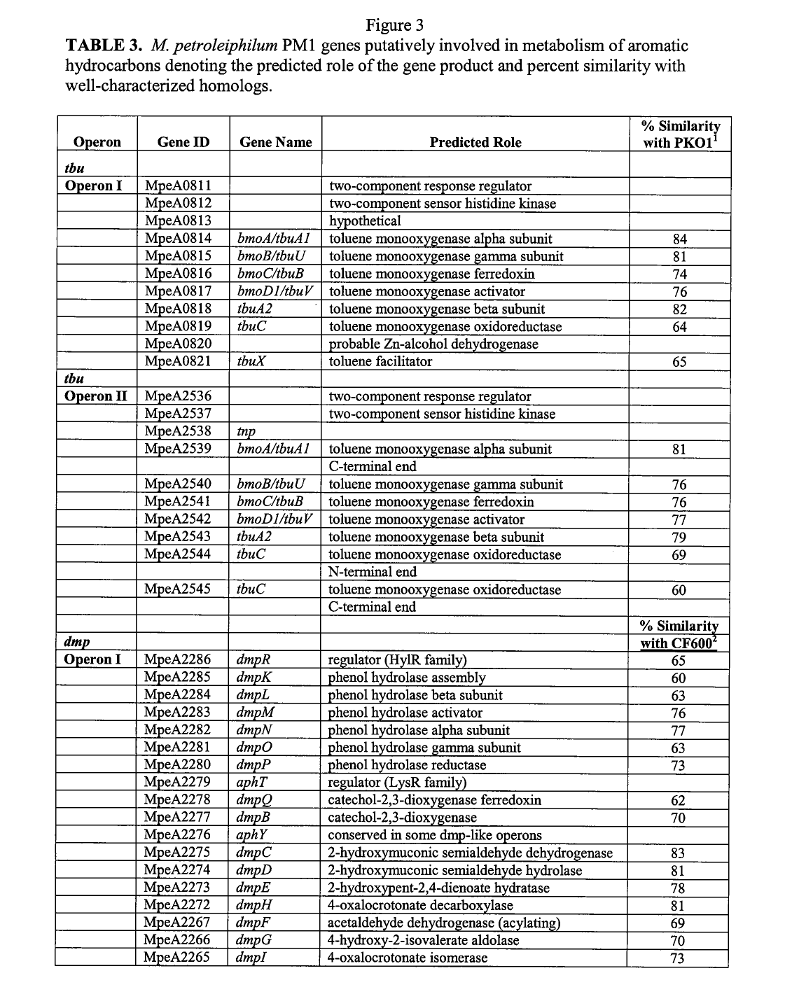

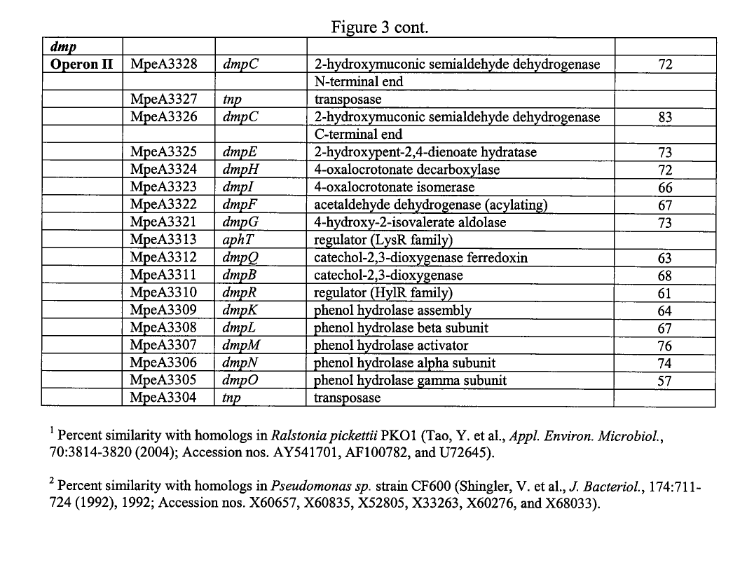

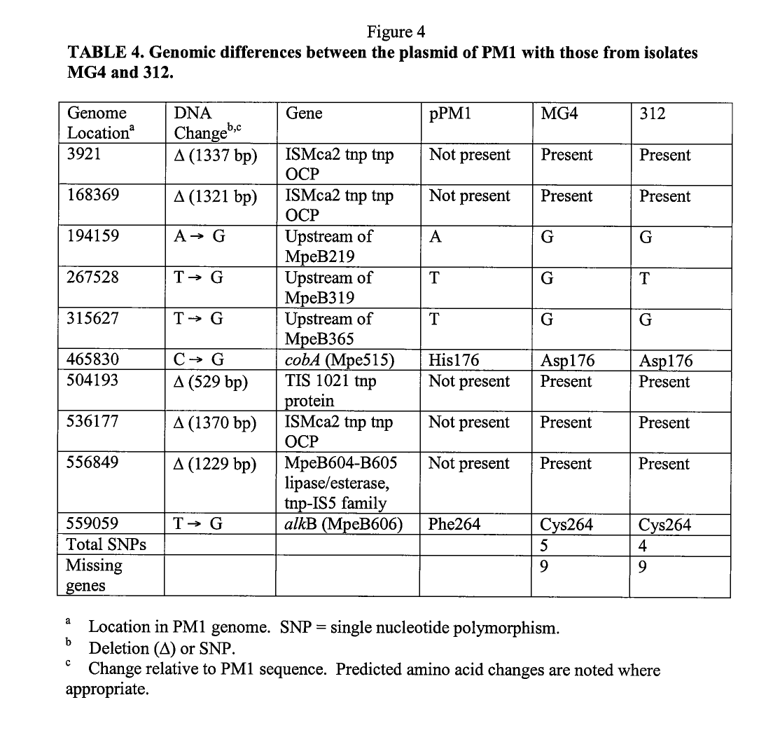

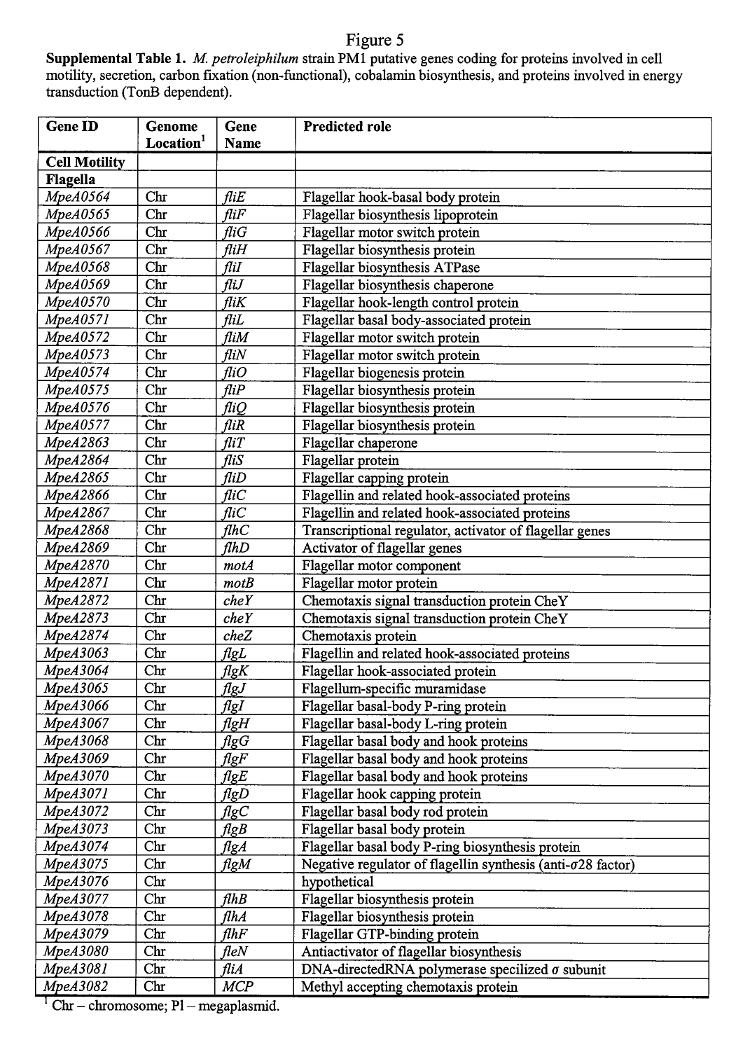

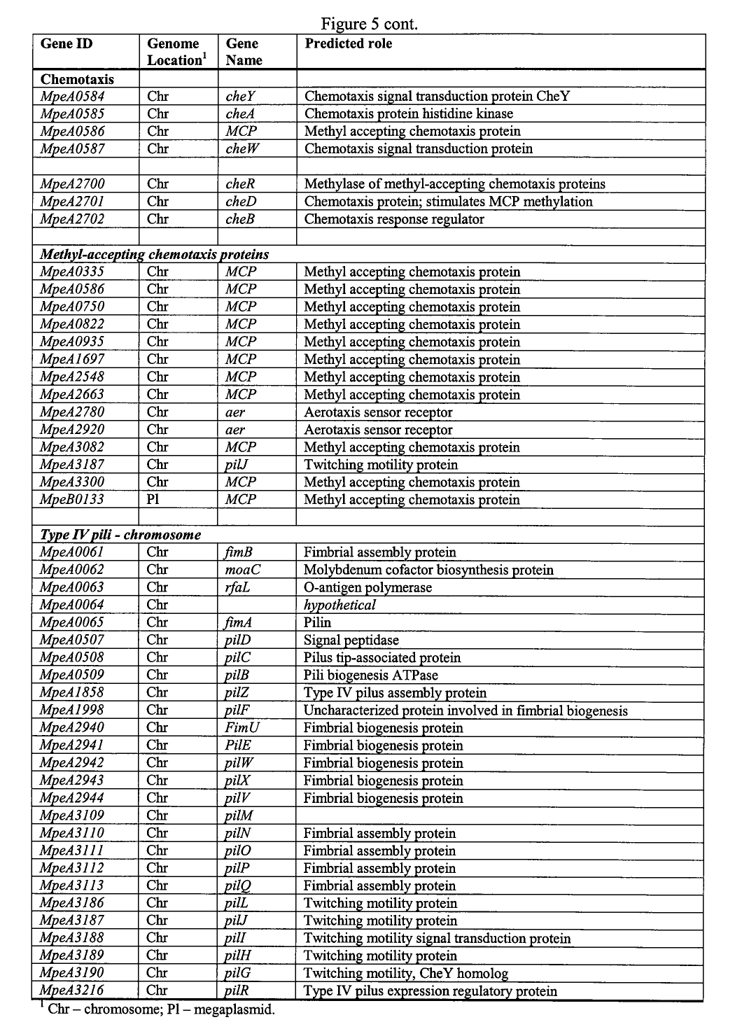

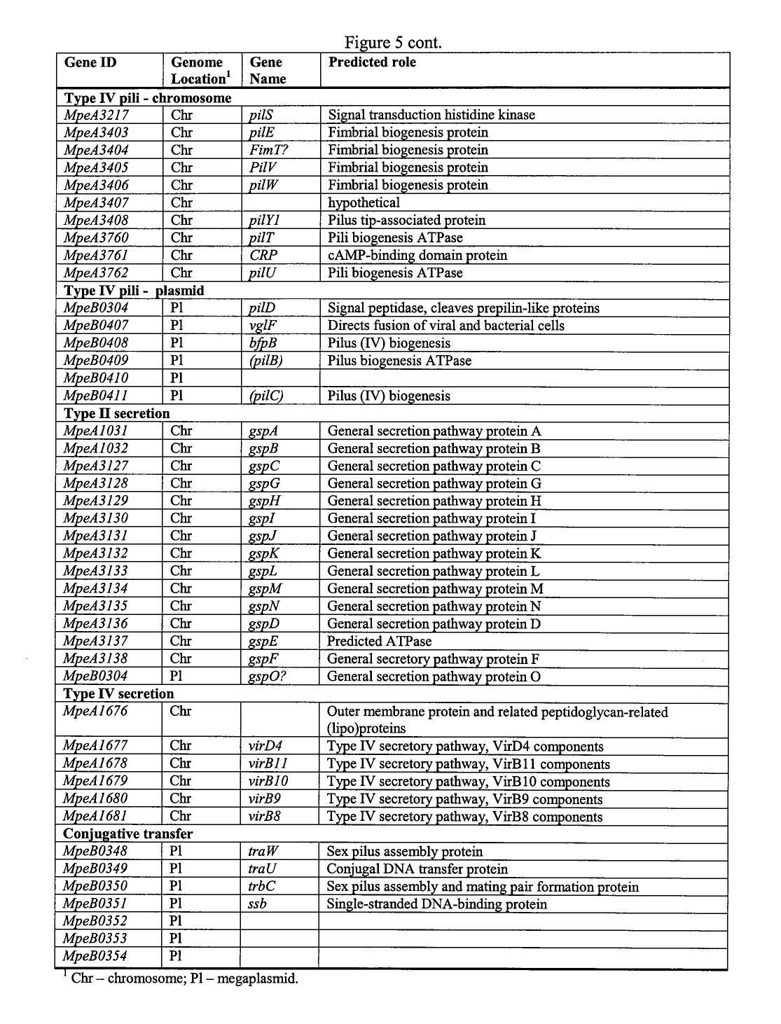

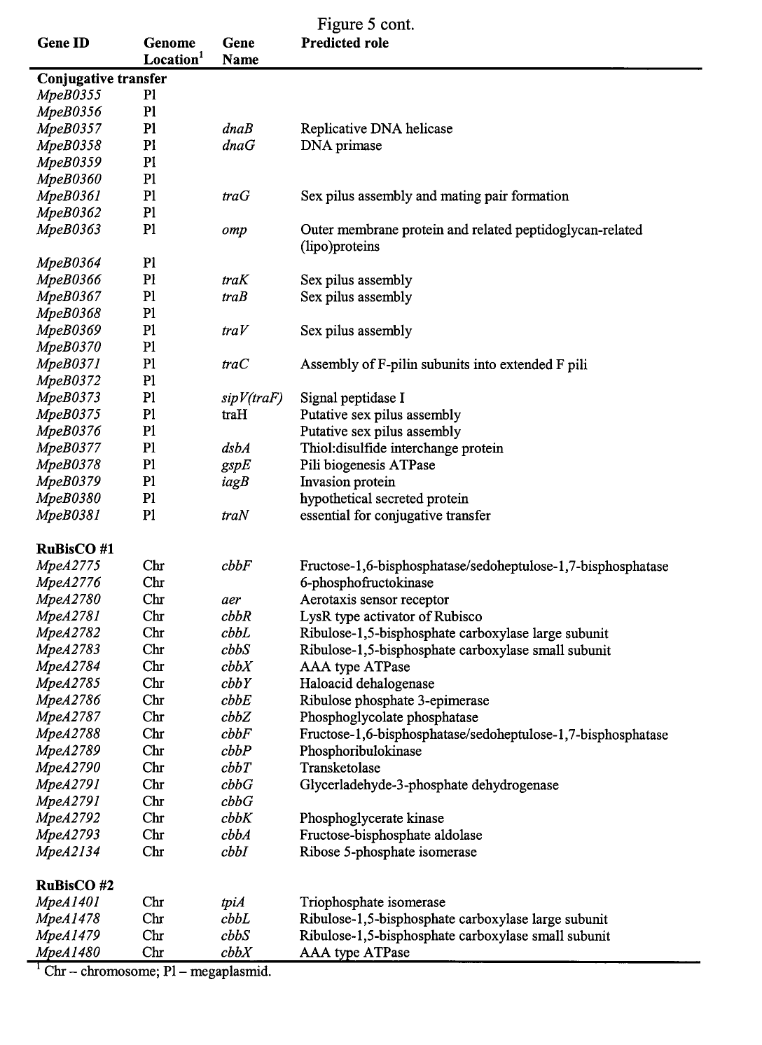

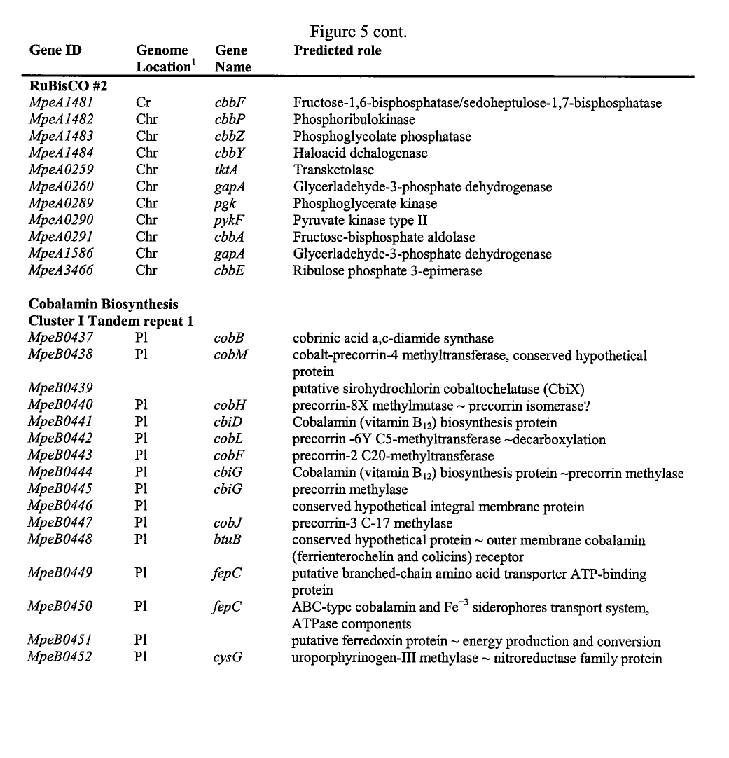

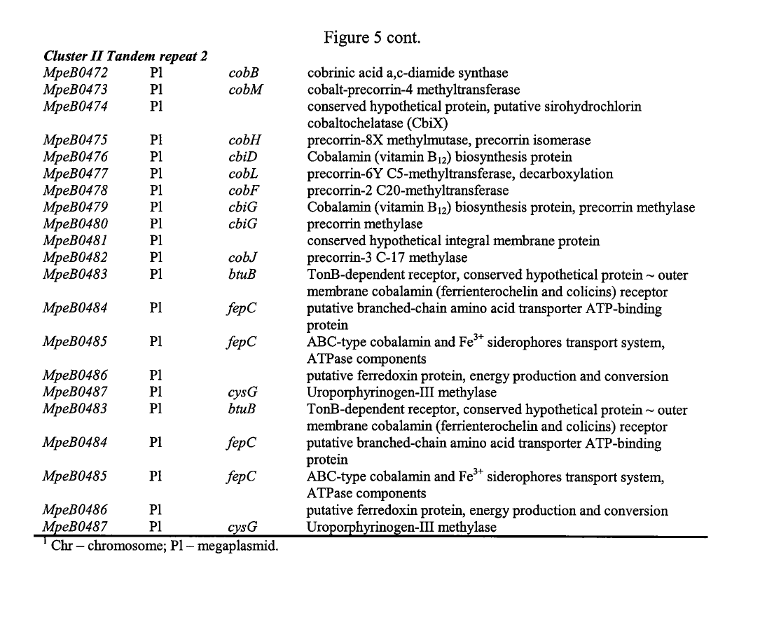

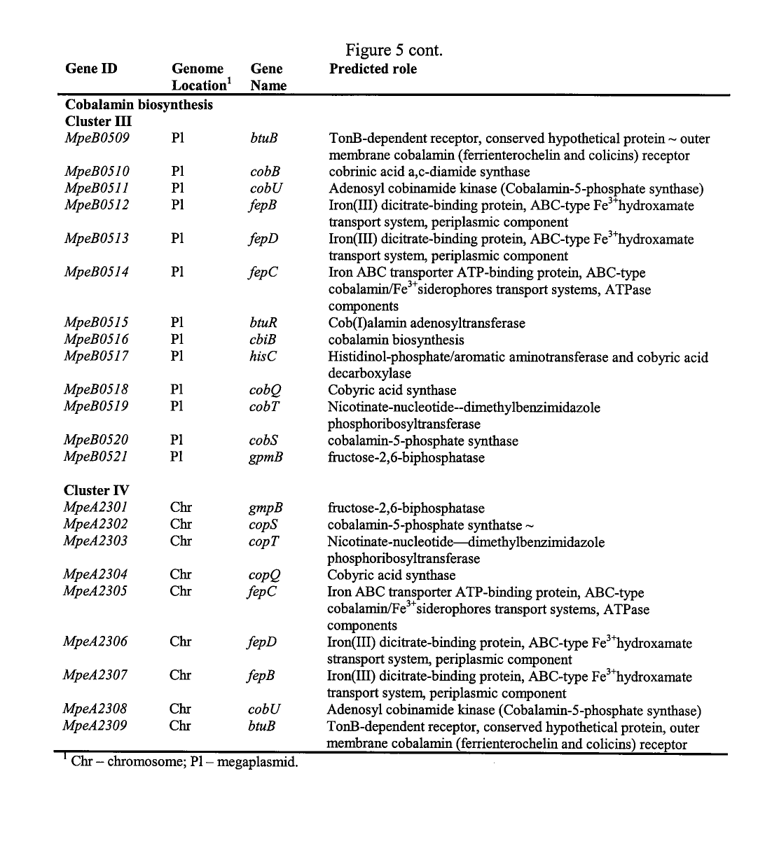

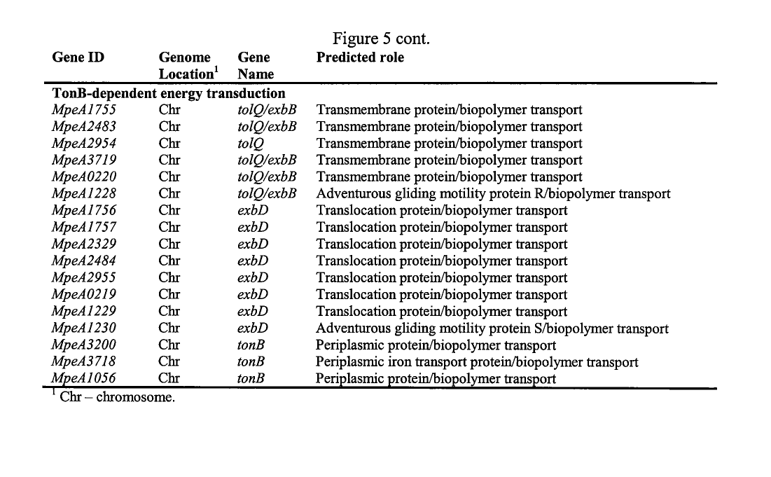

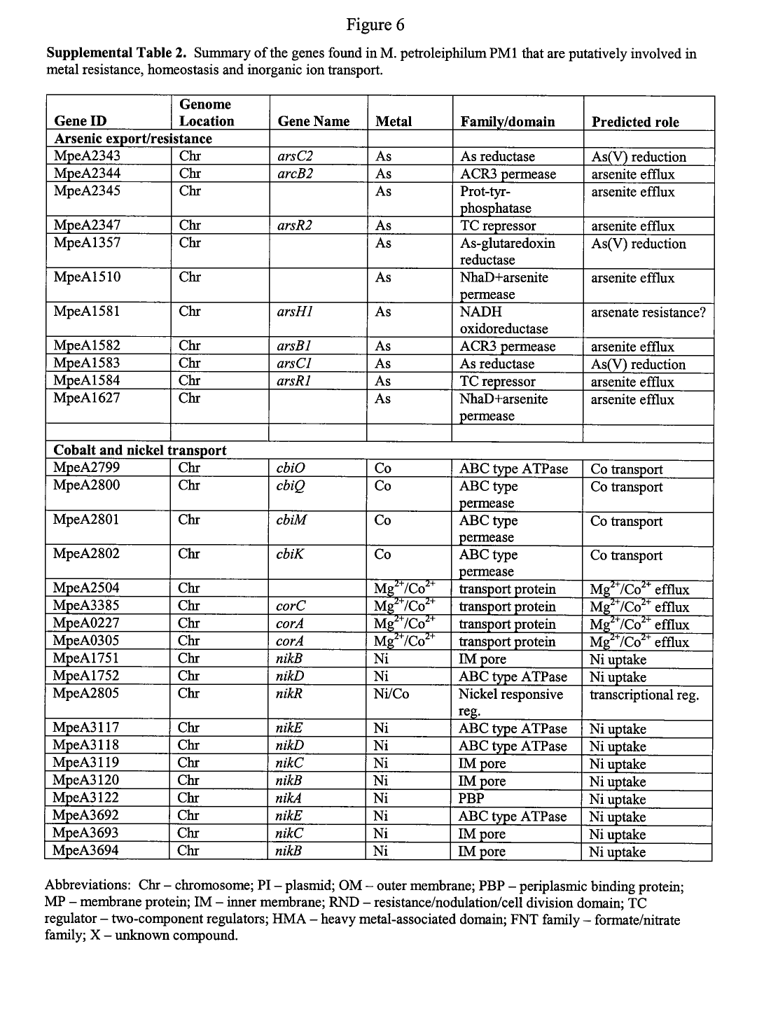

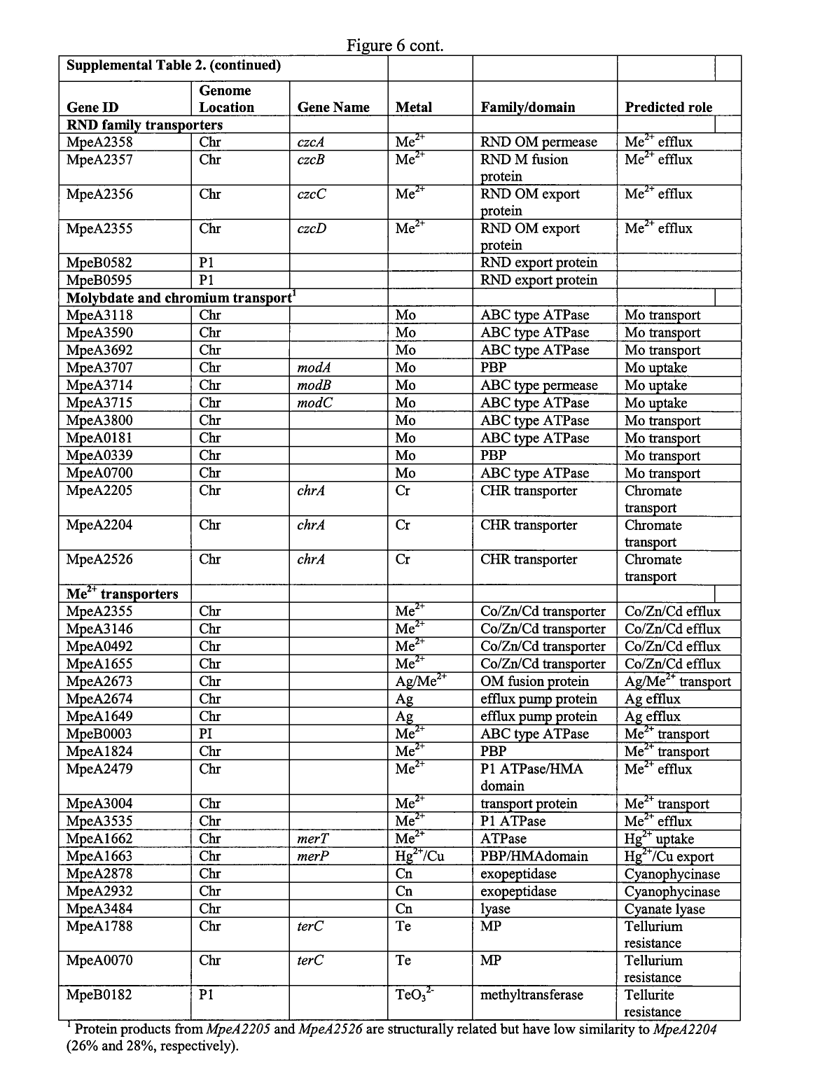

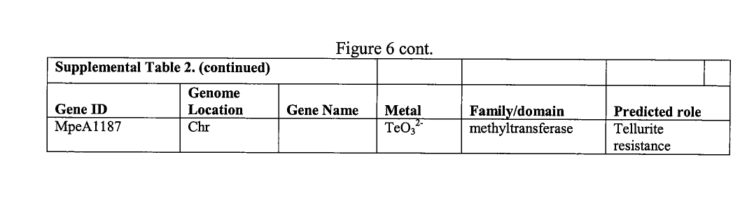

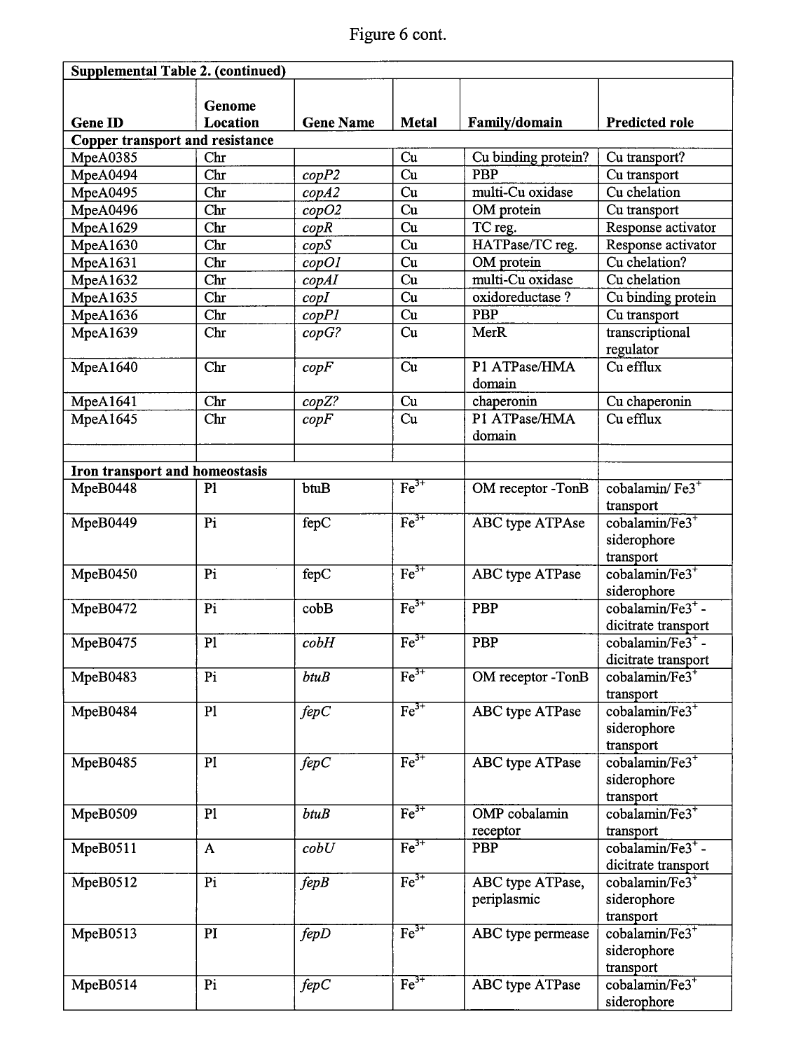

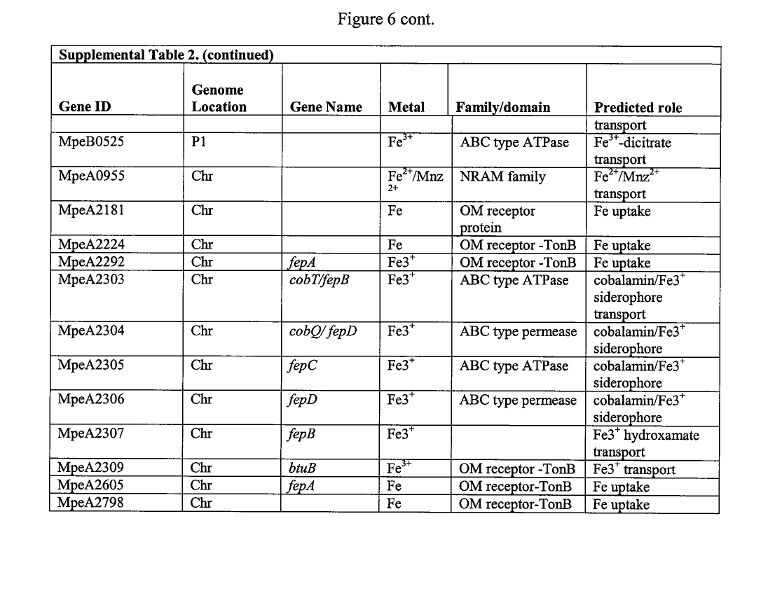

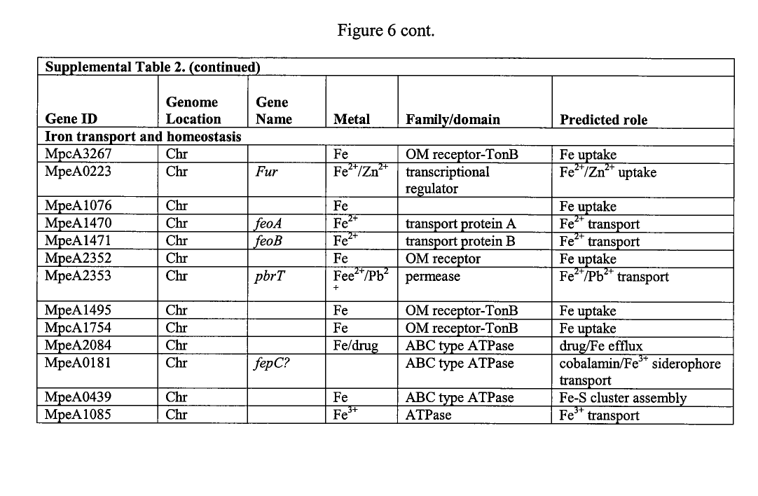

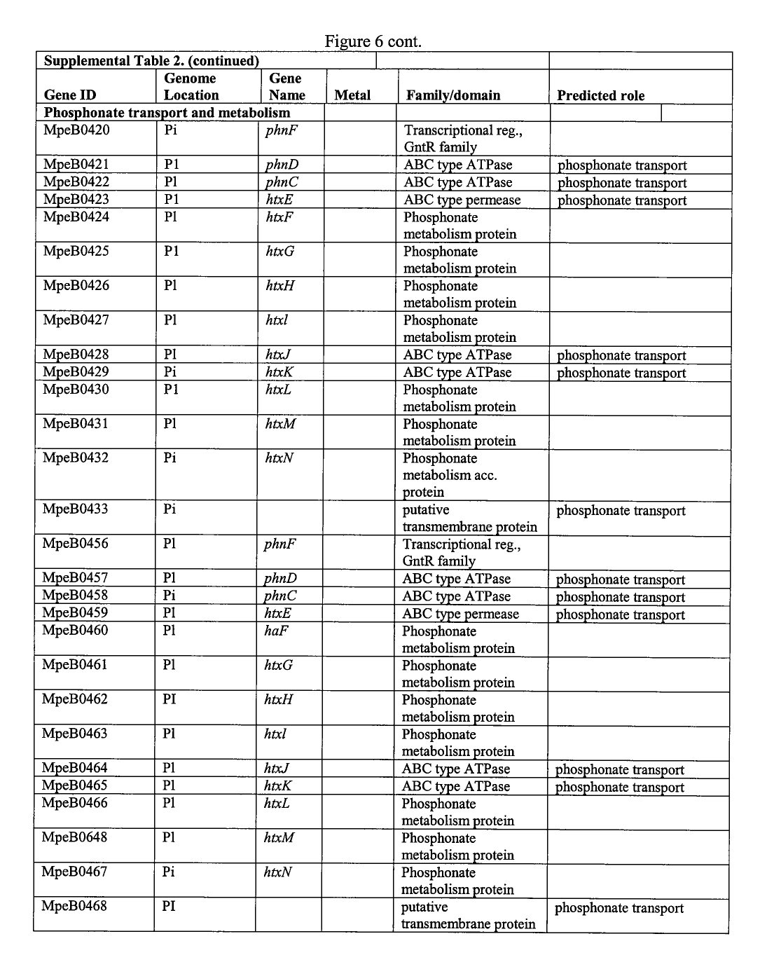

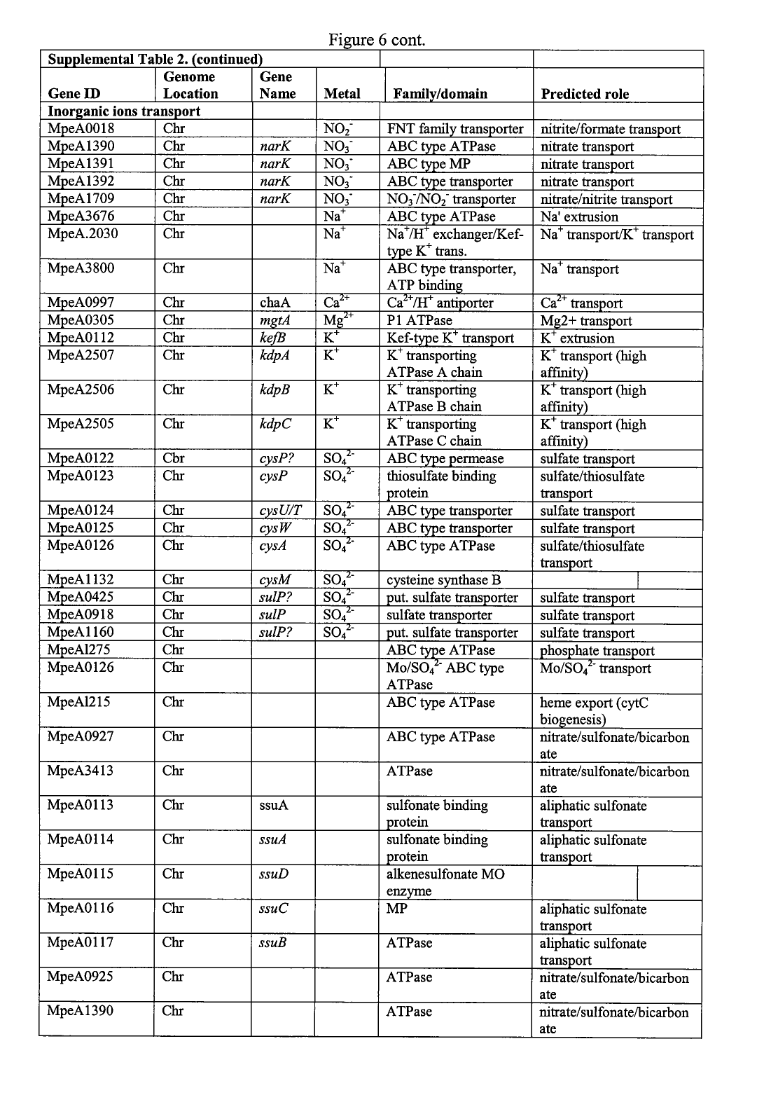

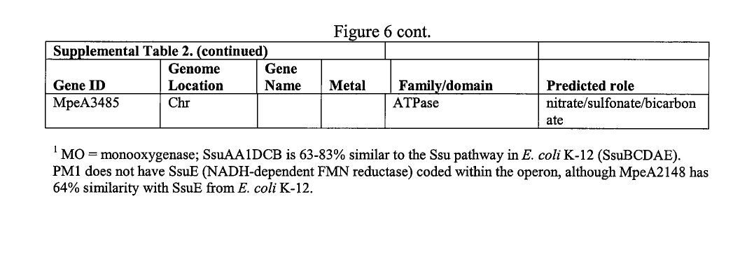

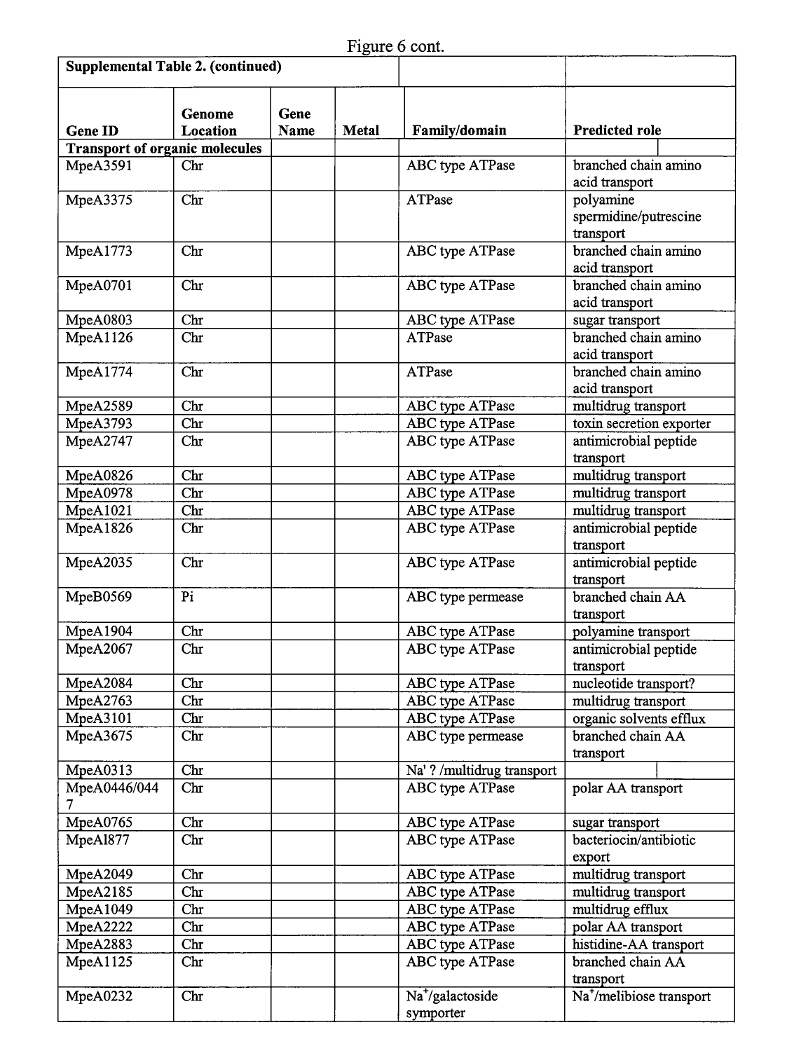

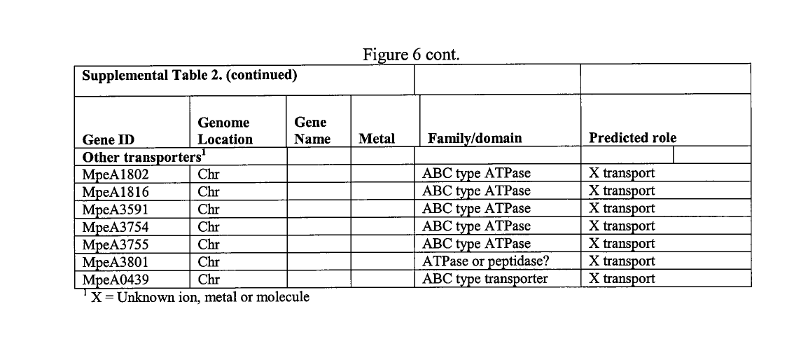

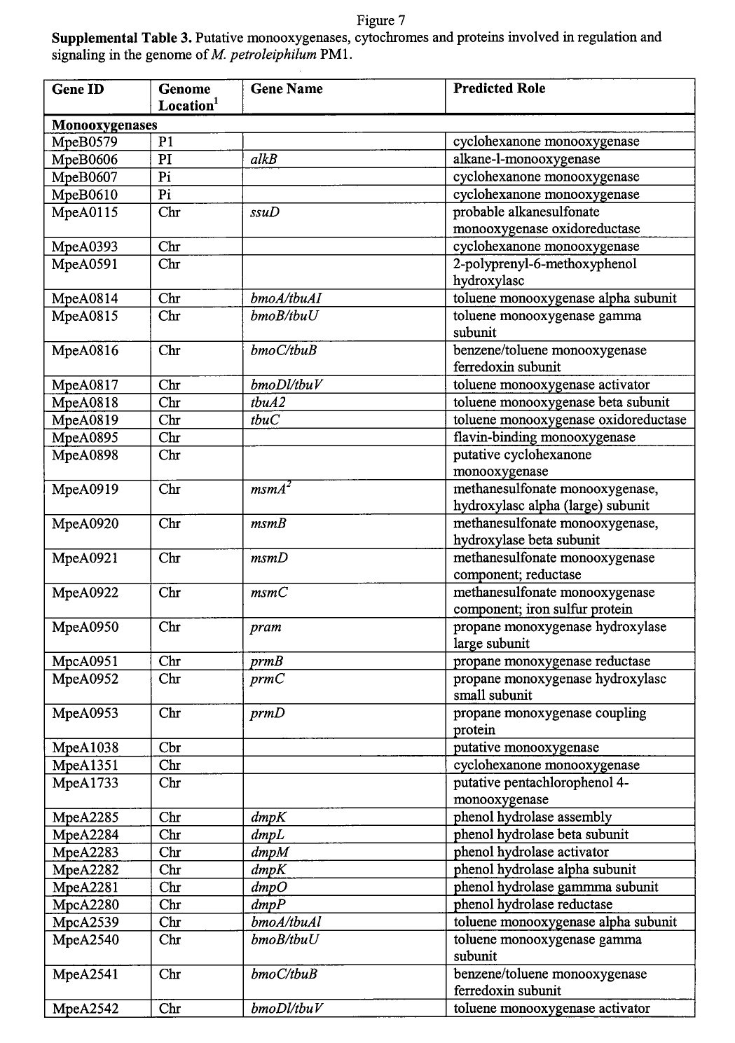

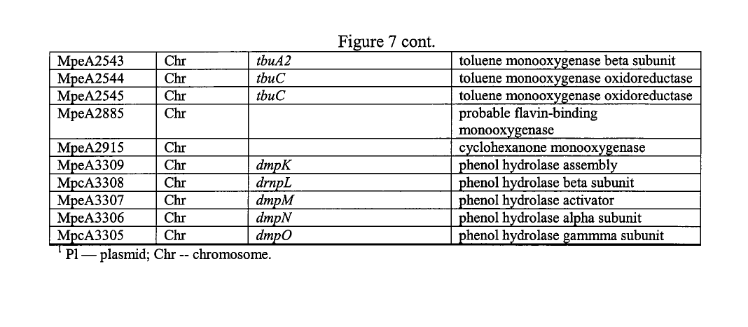

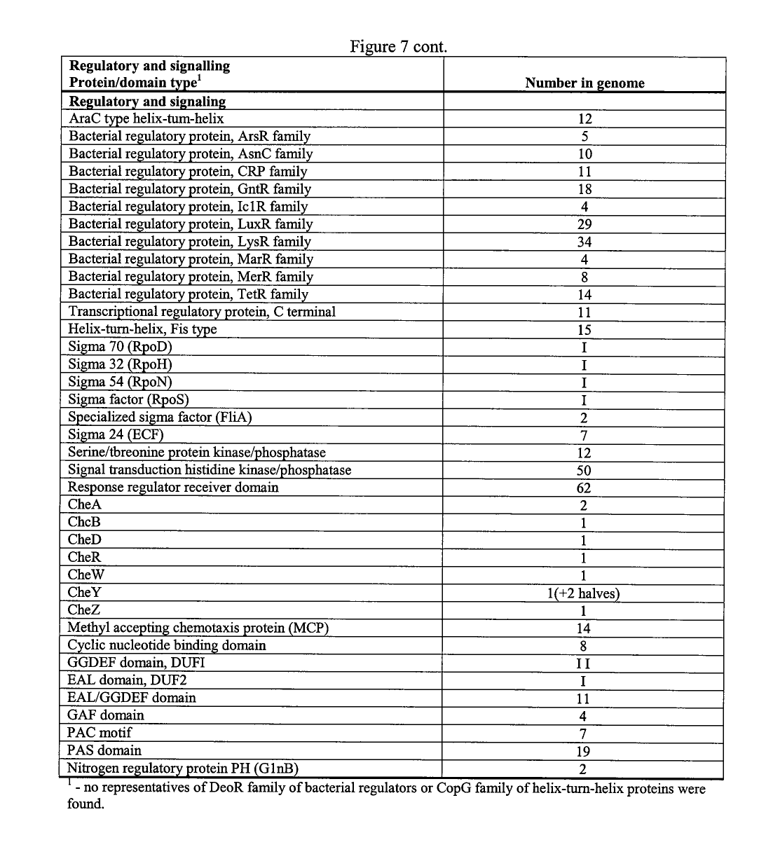

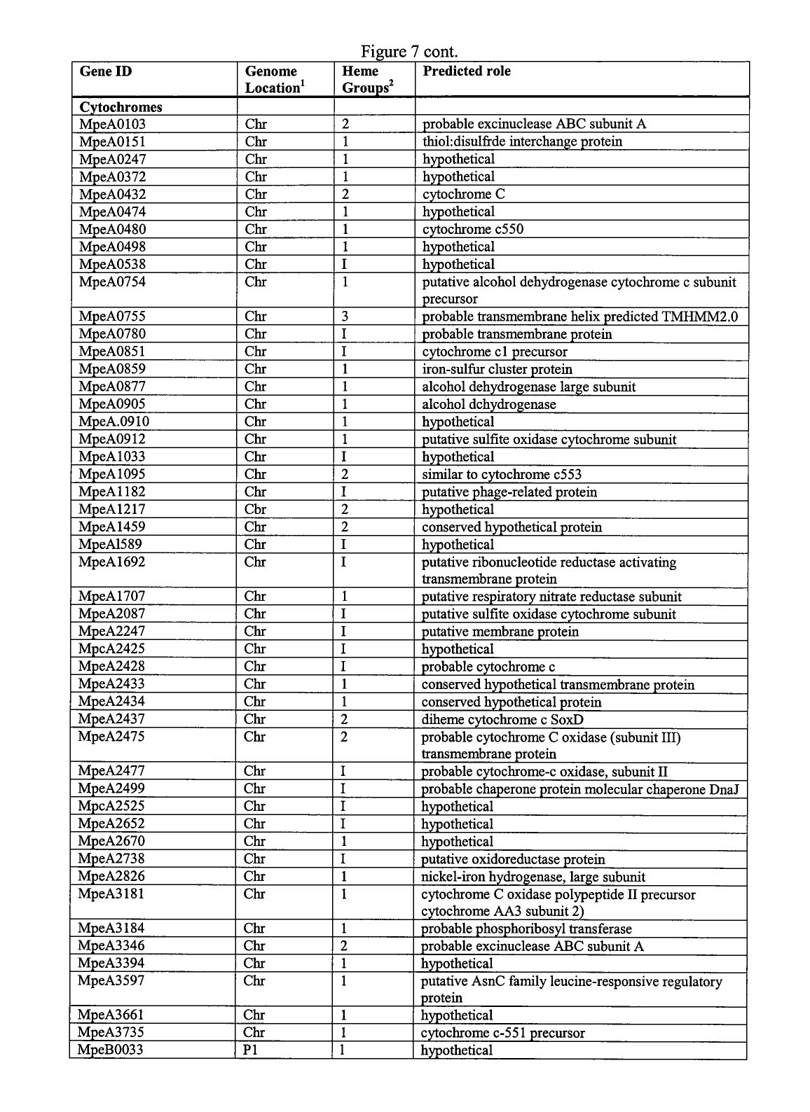

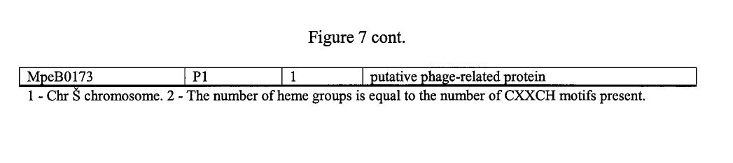

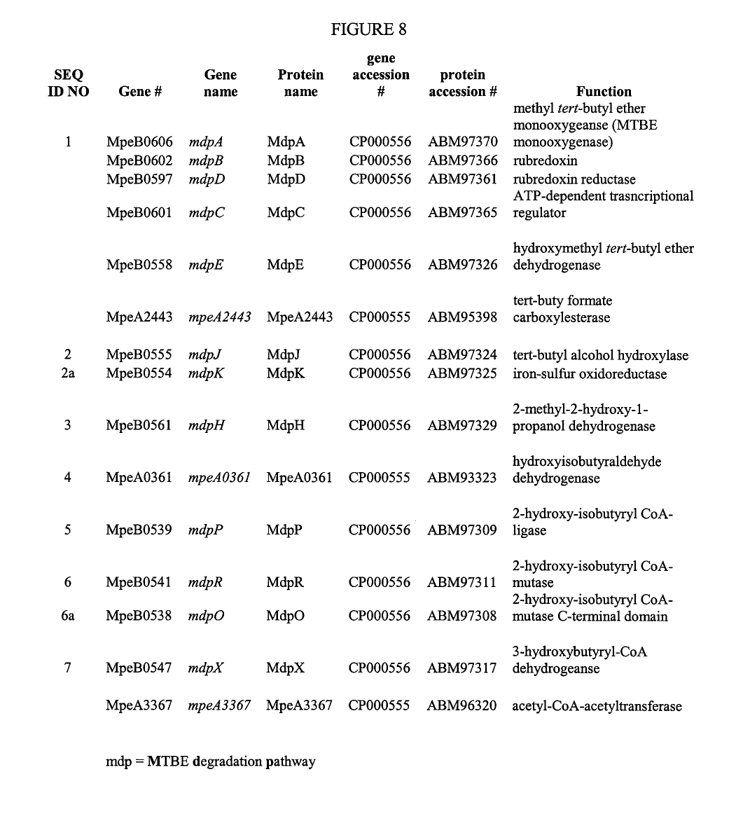

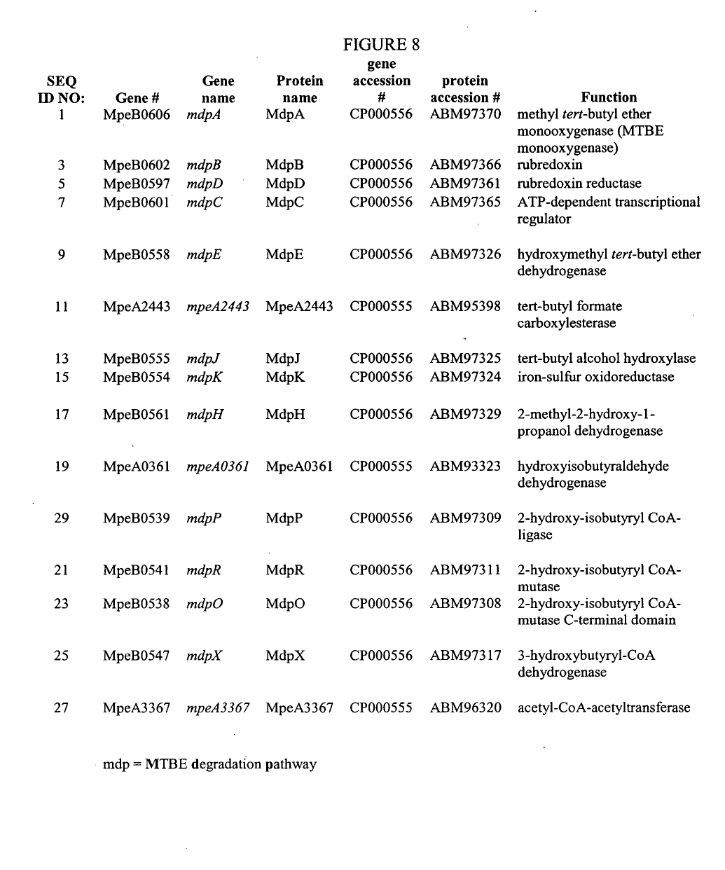

General genome features of chromosome and megaplasmid. The Methylibium petroleiphilum strain PM1 genome consists of a circular chromosome of 4,044,225 bp (FIG. 1a.), and a megaplasmid (pPM1) of 599,444 bp (FIG. 1b.) (Table 1). The genome encodes 4,477 putative CDSs, of which 964 are unique to PM1 based on BLASTP searches against NR. The pPM1-encoded proteins account for a disproportionately large number (382) of these unique genes. Of the remaining proteins, 2801 could be assigned a putative function based on the KEGG (Kyoto Encyclopedia of Genes and Genomes) database. Analysis of the top BLAST hits (against completed genomes in KEGG) revealed the closest homolog was most often found in other beta-proteobacterial genomes (2332), with the most hits (790) to Ralstonia solanacearum followed by Burkholderia pseudomallei (497) and Azoarcus sp. EbN1 (413). This distribution appears to reflect that of the chromosome: 2210, 589 and 364 to beta-, gamma-, and alpha-proteobacteria, respectively (Table 1). Interestingly, in contrast to the chromosome where beta- and gamma-proteobacteria account for 57.7% and 15.4% of its top BLAST hits respectively, the distribution of top hits between beta- (18.9%) and gamma- (15.6%) proteobacteria is nearly equivalent on the megaplasmid. The lower fraction of beta-proteobacteria-like CDSs in the megaplasmid is balanced by the large proportion of CDSs with no hits to KEGG genomes (47.7%) compared with the CDSs on the chromosome (9.9%). This surprising difference in the phylogenetic distribution of best hits together with the discrepancy in G+C content between the plasmid (66%) and the chromosome (69.2%) points to the likelihood that the plasmid was horizontally acquired; further evidence for this statement is provided by conservation of the megaplasmid in other phylogenetically similar MTBE-degrading bacteria (discussed in detail later). Analysis of Clusters of Orthologous Genes (COG) distribution (Tatusov, R. L. et al., Nucleic Acids Res., 28:33-36 (2000)) showed that the most abundant groups (excluding no COG or general function) were amino acid transport and metabolism (7.0%), energy production (6.4%), and transcription (6.3%) on the chromosome, and replication, recombination and repair (8.0%), coenzyme transport and metabolism (7.0%), and inorganic ion transport and metabolism (5.3%) on the plasmid.

The chromosome contains a single ribosomal rrn operon (16S-tRNA^{ala}-tRNA^{ile}-23S-5S) and all genes coding for ribosomal proteins. Structural RNA genes for SRP RNA, rnpB, and tmRNA were present. Forty-two tRNA genes, evenly distributed on the chromosome (with the exception of a few clusters of 2 or 3 tRNAs), correspond to 40 tRNA acceptors and can recognize all possible codons. A very unusual feature of pPM1 is that it contains a single large cluster of 27 additional tRNA genes (25 are redundant with those on the chromosome, the two others do not have clear anticodons). This is the first report of such a large tRNA gene island, and the first report of such a cluster on a plasmid. The role of this island in translation, in genome evolution, or in positive selection of the plasmid in this or other bacterial strains is unclear.

Cell motility, secretion and transport systems. M. petroleiphilum PM1 possesses the genes necessary for flagellar biosynthesis (for one polar flagellum), chemotactic response, type IV pili synthesis, the type II secretion pathway as well as several genes related to the Agrobacterium tumefaciens type IV secretion pathway (Supplemental Table 1). Type IV secretion mechanisms are often involved in pathogenesis. However, homologs to only three of the five core type IV secretion proteins (VirB9, 10, 11, not VirB4 or 7) (Backert, S. and T. F. Meyer, Curr. Opin. Microbiol., 9:207-217 (2006)) were identified, so it is unclear at present if PM1 possesses a functional type IV secretion pathway. PM1 likely moves both by flagellar-facilitated swimming and pili-facilitated twitching motility. Three copies of tra genes on pPM1 suggest that PM1 may be capable of conjugative transfer, a possibility currently under investigation. Thirteen chromosomal and one plasmid born methyl-accepting chemotaxis proteins (MCP's) allow PM 1 to respond to a range of environmental stimuli (Supplemental Table 1). As in other organisms (Zhulin, I. B., Adv. Microbial Physiol., 45:157-198 (2001)), MCP's in PM1 are found scattered throughout the genome. Only three MCP's are found within taxis operons—the pilG-L operon, the cheYA(MCP)W operon and the flg/flh gene cluster. The apparent mobility of MCP's, together with the fact that six PM1 MCP's appear to be paralogs, complicate function assignation. Nevertheless, there are five other MCP's in addition to those already mentioned whose gene environment may offer insight into their possible functions: two paralogous MCP's are located immediately downstream of and may be part of the same operon as the two toluene/benzene monooxygenase pathways; one of the aer-like MCP's is immediately downstream of, and in the same orientation as the LysR-type activator of the ribulose 1,5-bisphophate carboxylase/oxygenase (RuBisCO) operon, which is upstream of the two regulatory genes; one MCP may be co-transcribed with a gene showing similarity to the direct oxygen sensor dos of E. coli; and one MCP may be co-transcribed with a gene showing low percent similarity to bacteriophytochromes and motility sensors. Neighbor-joining analysis of the putative PM1 MCP's against their homologs showed that eight MCP's cluster close to MCP1-4 of E. coli MCPA-D of S. typhimurium, two appear related to the aerotaxis and energy sensor AER of E. coli, and one is very similar to the twitching motility protein PilJ of P. aeruginosa.

Strain PM1 has two sets of genes coding for form I RuBisCO, cbbL (mpeA1478 and mpeA2782) and cbbS (mpeA1479 and mpeA2783) and associated enzymes required for CO₂ fixation via the Calvin cycle (Supplemental Table 1); however, this activity has not been demonstrated for PM1. A thorough search of the PM1 genome revealed the absence of key enzymes from each of the three other known CO₂ fixation pathways: 2-oxoglutarate:ferredoxin oxidoreductase and ATP citrate lyase (reductive TCA cycle); the acetyl-CoA synthase/CO dehydrogenase (reductive acetyl-CoA pathway); malonyl-CoA reductase and propionyl-CoA synthase (3-hydroxypropionate cycle) (Atomi, H., J Biosci. Bioengineer, 94:497-505 (2002); Hugler, H. et al., Arch. Microbiol., 179:160-173 (2003)). This strain possesses several ABC transporters for transport of inorganic ions such as nitrate, sulfate, magnesium, potassium, phosphate, phosphonate, as well as amino acids, branched chain amino acids, carbohydrates, long chain fatty acids, dipeptides/oligopeptides, polyamines, and antibiotics (Supplemental Table 2). In addition, putative regulatory/signaling proteins, and cytochromes (based on CXXCH motifs) have been identified (Supplemental Table 3).

Repeated elements. The genome has a number of complex repetitive elements, including eight families of insertion sequences (ISmp1-8) (up to 12 copies) and two large genomic segments (29 and 40 kb) flanked by IS elements that have undergone what appears to be recent duplications. The two replicons do not equally share the repeated insertion elements; five of the eight families are located only on the chromosome and one family is strictly found on the plasmid. The distribution patterns of the IS elements, lends support to the dissimilar phylogenetic distribution of best KEGG hits among sequenced genomes and strengthens the notion of the megaplasmid's recent acquisition.

Parallel copies of ISmp8 flank two tandem copies of a 29-kb repeat, each consisting of two operons involved in phosphonate and cobalamin metabolism. The phosphonate operons (PhnFDC-HtxFGHIJKLMN) include putative C—P lyase subunits 54-83% similar to those of Pseudomonas stutzeri WM88 (White, A. K. and W. W. Metcalf, J. Bacteriol., 186:4730-4739 (2004)) (Supplemental Table 2). The Htx and Phn C—P lyases support growth on methylphosphonate or additional alkylphosphonates, respectively; growth on these substrates is not yet known for PM1. Also contained in the repeat are cobalamin (vitamin B₁₂) synthesis genes encoding the conversion of uroporphyrinogen III to cobinamide and the synthesis of dimethylbenzimidazole (DMB) in the aerobic pathway for cobalamin biosynthesis (mpeB437-453, B472-488) (Supplemental Table 1). Downstream of the tandem repeat are the remaining genes (mpeB509-522) for the covalent linkage of DMB, cobinamide and a phosphoryl group to complete the cobalamin synthesis pathway. Genes coding for the anaerobic pathway of cobalamin biosynthesis (cbi genes) are also present in the cob clusters, however, a complete pathway is lacking. PM1 also lacks a cobG encoding the monooxygenase that converts precorrin-3 to precorrin-4 in the aerobic pathway, however one or more of the multiple copies of cbiG (mpeB479, 480, 444, 445) may code for a functional enzyme that performs this reaction without oxygen (Rodionov, D. A. et al., J. Biol. Chem., 278:41148-41159 (2003)). Cobalt, and cobalamin (vitamin B₁₂) have been shown to enhance PM1's ability to grow on and degrade MTBE and its primary metabolite, tert-butyl alcohol (TBA) (K. Hristova, unpublished data) so it is not surprising that multiple copies of genes involved in cobalamin synthesis are present in PM1 including tandem repeats of cob and cbi genes. Recently, Rohwerder et al. (Rohwerder, T. et al., Appl. Environ. Microbiol., 72:4128-4135 (2006)) showed that cobalamin synthesis affected the growth rate on the MTBE metabolites, TBA and 2-hydroxyisobutyrate (2-HIBA) for a beta-proteobacterial MTBE-degrading strain that was phylogenetically similar to PM1 (95.6% identical based on 16S rDNA sequence). In this strain, cobalt or cobalamin was necessary for activity of an enzyme, isobutyryl-CoA mutase involved in metabolism of 2-HIBA (Rohwerder, T. et al., Appl. Environ. Microbiol., 72:4128-4135 (2006)), and 99% identical homologs to this two-component mutase are present in PM1 (mpeB538/541). As mentioned, a relatively large percentage of predicted proteins on the plasmid (7.0%) belong to the COG category for coenzyme transport and metabolism. A cluster of ethanolamine utilization (eut) genes (mpeB0499-502) found between the tandem repeats and third cobalamin cluster on the plasmid encode putative proteins 48-85% similar to EutJEMN from the cobalamin-dependent ethanolamine utilization pathway of S. typhimurium (Kofoid, E. et al., J. Bacteriol., 181:5317-5329). The latter two proteins are homologs of carboxysome shell proteins (CcmKL). While S. typhimurium also contains the ethanolamine lyase subunits and regulator (EutBC and EutR) in its eut operon, in PM1 these genes (mpeA2417-8, mpeA2415) are on the chromosome.

The largest (40 kb) repeated element is present on both the plasmid and chromosome, and encodes a putative PinR-like site-specific recombinase, a replicative DNA helicase, 2 putative spoJ-like transcriptional regulators or plasmid partitioning proteins, a spoIIIE-like DNA translocase, a tellurite resistance protein, and many hypothetical products. The presence of the repeat on both replicons suggests a recent duplicative transfer event. Though the types of genes found in this region suggest a plasmid origin, both copies interrupt similar but non-identical copies of dcd genes (dCTP deaminase), and the direction of duplicative transfer remains to be proven. Since this duplication, the 40-kb repeat on pPM1 has been interrupted by a transposase between genes mpeB0184 and mpeB0187.

Heavy metal tolerance and metal homeostasis. One interesting outcome of the genome analysis is evidence of PM1's potential resistance to heavy metals suggesting promise in using the organism to treat sites containing mixed wastes. Arsenic extrusion in PM1 is probably mediated by arsRBC present in two copies on the chromosome that are 58-81% similar to each other (mpeA1581-1584, arsHBCR; mpeA2343-4, A2347, arsCB and arsR). While some other bacteria have five-gene operons (arsRDABC) and use the ArsAB pump, PM1 probably extrudes arsenite by a carrier protein, ArsB alone energized by membrane potential (Rosen, B. P., FEBS Lett., 529:86-92 (2002)), although resistance to arsenic oxyanions needs to be evaluated. ArsC encodes an arsenic reductase responsible for the transformation of As(V) into As(III) and ArsR is a transcriptional repressor that responds to As(III) and Sb(III) (Rosen, B. P., FEBS Lett., 529:86-92 (2002)). The function of the fourth gene product, ArsH, found in several bacteria (Yersinia enterocolitica, Acidothiobacillus ferrooxidans, Pseudomonas aeruginosa, P. putida KT2440) still remains unclear (Butcher, B. G. et al., Appl. Environ. Microbiol., 66:1826-1833 (2000); Ryan, D. and E. Colleran, Plasmids, 47:234-240 (2002)). Three chromosomal copies of chrA (mpeA2204, mpeA2205 and mpeA2526), belonging to the CHR family of transporters, may mediate chromate resistance in PM1. One copy of ChrA (mpeA2204) is 63 and 61% similar to its homolog in Dechloromonas aromatica RCB and P. putida KT2440, respectively, although a homologous chromate reductase (ChrR; Jimenez, J. I. et al., Environ. Microbiol., 4:824-841 (2002)) was not evident in PM1.

Genome analysis revealed 15 copper resistance genes in a large cluster (˜14.4 kb at positions 1760297-1775675) on the PM1 chromosome with structural analogy to the plasmid-mediated (pMOL30) copper resistance cluster copOAIPRSFG in Ralstonia metallidurans (Mergeay, M. et al., FEMS Microbiol. Rev., 27:385-410 (2003)) (Supplemental Table 2). The CopOAIP-CopRS cluster in PM1 is likely involved in the efflux of periplasmic copper (analogous to cop system of P. syringae, R. metallidurans, and R. solanacearum, and the cos system of E. coli [Cervantes, C. and F. Gutierrez-Corona, FEMS Microbiol. Rev., 14:121-137 (1994)]), whereas, the efflux of cytoplasmic copper is mediated by a P1-ATPase, CopF1F2 (analagous to R. metallidurans CopF) (Mergeay, M. et al., FEMS Microbiol. Rev., 27:385-410 (2003)). The genome of PM1 also has a putative chemiosmotic antiporter efflux system similar to CzcCBA of R. metallidurans, conferring resistance to Cd, Zn and Co (Mergeay, M. et al., FEMS Microbiol. Rev., 27:385-410 (2003)). In addition to copF, there are two other genes encoding putative metal-transporting P1-type ATPases, mpeA2479 and mpeA3535. Additional proteins potentially involved in metal transport include NikBCDE for nickel (mpeA3117-3120), CbiOQMK for cobalt and nickel (mpeA2799-2802), and ModABC (mpeA3707, mpeA3714, mpeA3715) for molybdenum uptake (Supplemental Table 2).

Ferric iron has also been shown to enhance PM1's ability to grow on and degrade MTBE and TBA (K. Hristova, unpublished data) so it is likely that active transport of iron is of particular importance. As with other Gram-negative bacteria, PM1 acquires its iron supply via Fe³⁺-siderophores. Fep genes, which function in the synthesis of polypeptides required for uptake of ferric enterobactin were identified inside each of the four cob operons in PM1 (Supplemental Table 1). Polaromonas sp., R. ferrireducens, and M. flagellatus all have iron transport genes either within or in close proximity to their cob operons (data not shown). Minimally, FepABC are required for ferric enterobactin uptake. MpeA2292 and mpeA2605 have been annotated asfepA, coding for an outer membrane receptor for an iron siderophore, however it is possible that btuB genes located near the febBDC genes are also involved in iron assimilation. The TonB-dependent energy transduction complex (tonB, exbB or tolQ, and exbB; Supplemental Table 1.) coded on the chromosome likely provides the mechanism for active transport of iron siderophores and cobalamin across the outer membrane (Braun, V. and M. Braun, FEBS Lett., 529:78-85 (2002); Higgs, P. I. et al., J. Bacteriol., 180:6031-6038 (1998)). The PM1 genome encodes about 39 putative proteins involved in iron transport and homeostasis, which implies the importance of iron in its physiology.

Methylotrophy. Methylotrophic metabolism of PM1 is of great interest because formaldehyde and formate are common intermediates of both methanol and MTBE or TBA oxidation by PM1 and other degraders (Butcher, B. G. et al., Appl. Environ. Microbiol., 66:1826-1833 (2000); Piveteau, P. et al., Appl. Microbiol. Technol., 55:369-373 (2001)). M petroleiphilum PM1 is capable of aerobic growth on methanol, formate, and succinate. Unlike other methylotrophic beta-proteobacteria, PM1 grows on MTBE, toluene, benzene, ethylbenzene, and dihydroxybenzoates as sole carbon sources (Nakatsu, C. H. et al., J. Sys. Evol. Microbiol., 56:983-989 (2006); Piveteau, P. et al., Appl. Microbiol. Technol., 55:369-373 (2001)). PM1 possesses genes for the serine cycle and methylotrophy scattered in several different clusters on its chromosome (Table 2). The strain does not grow on methylamine (K. Hristova, unpublished data), lacks a gene encoding the methylamine dehydrogenase (MADH) large subunit, and likely lacks MADH activity. Despite the ability of PM1 to grow on methanol, its genome lacks true homologs of mxaF and mxaI, known genes coding for the methanol dehydrogenase (MeDH) large and small subunits, present in several methylotrophs known to date. PM1 contains a MeDH-like cluster XoxF-J (mpeA3393-5) that is present in Methylobacterium extorquens AM1 (Chistoserdova, L. and M. E. Lidstrom, Microbiol., 143:1729-1736 (1997)), which also contains the true mxaF cluster. Comparative sequence analysis of the product of gene mpeA3393 revealed high similarity to MxaF (74% to M. extorquens AM1) and the XoxF homolog present in several non-methylotrophs (77% to Burkholderia fungorum). Based on phylogenetic analysis, MpeA3393 clusters with the MxaF homologs of unknown function from other methylotrophic and non-methylotrophic Rhizobia and Burkholderia spp., while true MeDH large subunits (MxaF) cluster together and are distinct from the MxaF homologs.

A cytochrome c-555 (mpeA3394) 56% similar to the C_{H} cytochrome of M. capsulatus Bath (electron donor to the oxidase in methylotrophic bacteria [Afolabi, P. R. et al., Biochem., 40:9799-9809 (2001)]) is found adjacent to the mpeA3394. A putative MxaJ/XoxJ (mpeA3395) shows 54% similarity with XoxJ from Paracoccus denitrificans and 42% similarity with MxaJ from M. capsulatus Bath. Five genes (mpeA3829, 2585-8) are involved in the biosynthesis of pyrroloquinoline quinone (PQQ), a cofactor of MeDH as well as quinoprotein ethanol dehydrogenase. A cluster of genes required for MeDH synthesis, mxaLKCASR (mpeA3273-3278) is also present. To date, none of the gene clusters containing the XoxF homolog have been shown to be involved in methanol oxidation. Therefore, it is possible that a new enzyme is responsible for this function in the beta-proteobacterium M. petroleiphilum PM1.

Three different formate dehydrogenases are present in the PM1 genome, with homologs to M. extorquens and M. capsulatus Bath. The function of the tungsten-dependent formate dehydrogenase fdh1 (mpeA0337-339), NAD-linked formate dehydrogenase fdh2 (mpeA3708-12), and cytochrome-linked formate dehydrogenase fdh3 (mpeA1170-71, 1173) for energy generation during growth on C₁ substrates or for MTBE oxidation needs to be further explored. MpeA3377 coding for a putative ABC-type tungstate transport system permease links gene clusters fdh1 and fdh2 (Table 2). The fdh2 genes in PM1 have the same gene arrangement and significant sequence identity (52-81%) to the NAD-dependent formate dehydrogenase cluster fdsGBACD of Ralstonia eutropha (Oh, J.-I. and B. Bowien, J. Biol. Chem., 273:26349-26360 (1998)). Pathways involved in metabolism and detoxification of formaldehyde, a central intermediate of both methanol and MTBE degradation by PM1 and other strains (Hara, A. et al., Environ. Microbiol., 6:191-197 (2004); Oh, J.-I. and B. Bowien, J. Biol. Chem., 273:26349-26360 (1998)), may also function in MTBE metabolism. PM1 has two pathways for formaldehyde oxidation to CO₂, an H₄ MPT-linked metabolic module that includes an archaeal-like gene cluster and an H₄F-linked metabolic module. Recently, phylogenetic analysis of a subset of bacterial and archaeal H₄MPT-linked C₁, transfer genes placed PM1 sequences with other described beta-proteobacteria (Kalyuzhnaya, M. G. et al., J. Bacteriol., 187:4607-4614 (2005)).

Fuel hydrocarbon degradation pathways. PM1 contains an operon (mpeA0814-0821) likely encoding for conversion of benzene to phenol (and catechol), and toluene to methylphenol (and methylcatechol) that is 62-74% similar to the benzene monooxygenase pathway in P. aeruginosa JI104 (BmoA-D1) (49) and 50-71% similar to the toluene para-monooxygenase (TpMO) pathway in Ralstonia pickettii PKO1 (TbuA1UBVA2CX) (Tao, Y. et al., Appl. Environ. Microbiol., 70:3814-3820 (2004)) (Table 3). A second operon (mpeA2539-2547) is 55-74% identical to the first operon, however, it likely does not yield a functional monooxygenase since the TbuA1 homolog is interrupted by a transposon insertion and the TbuC homolog is a pseudogene. Both operons have two-component response regulator-sensor histidine kinases upstream and divergently transcribed (mpeA0811-812; mpeA2536-2537) although mpeA2537 may be truncated due to the transposon insertion. MpeA821 encodes a putative TbuX (65% similar to that in PKO1), an outer membrane protein regulated by TbuT and involved in toluene uptake (Kahng, H.-Y. et al., J. Bacteriol., 182:1232-1242 (2000)). The BMO pathway has been implicated in benzene and toluene degradation (Kitiyama, A. et al., J Ferment. Bioeng., 82:421-425 (1996)), as has the TpMO pathway (Tao, Y. et al., Appl. Environ. Microbiol., 70:3814-3820 (2004)). In addition to benzene and toluene, PM1 has been shown to degrade o-xylene (Deeb, R. A. et al., Am. Chem. Soc., 219:ENVR 228 (2000)), although the biochemical pathway has not been elucidated to date. It is likely that m- and p-xylene can also be metabolized via the toluene monooxygenase (TMO) pathway of PM1 as described for PKO1 and other bacteria (Fishman, A. et al., Biocat. Biotrans., 22:283-289 (2004)).

M. petroleiphilum PM1 grows on phenol, and two distinct clusters of dimethylphenol (dmp)-like genes are present (mpeA2265-67, 2272-86; mpeA3305-13, 3321-25) although the latter lacks the key structural gene dmpP so may not yield a functional phenol hydroxylase (PH). Gene products from the first cluster dmpRKLMNOPQBCDEHFGI are 60-83% similar to those on pVI150 in Pseudomonas sp. strain CF600 (Shingler, V. et al., J. Bacteriol., 174:711-724 (1992)), including a multi-component PH, catechol 2,3-dioxygenase and the meta-cleavage pathway for catechol (Table 3). The second operon has transposon insertions inside dmpC and adjacent to dmpO. The PH subunits for the two operons are 44-69% similar. The DmpR homologs (mpeA3310, A2286) are similar to TbuT (69 and 65%) and may regulate TMO, PH and the meta-cleavage genes, since TbuT was shown to regulate these genes in PKO1 via separate promoters (Byrne, A. M. and R. H. Olsen, ThuT. J. Bacteriol., 178:6327-6337 (1996)). However these operons are located together in PKO1, whereas, they are quite distant in PM1. PM1 can grow on phenol, and based on the presence of a complete dmp operon, it can likely degrade alkylphenols as well, although it is not clear whether PH is essential for methylphenol degradation (as described for P. stutzeri OX1 [Cafaro, V. et al., Appl. Environ. Microbiol., 70:2211-2219 (2004)]) or whether the TMO alone is capable of converting toluene to methylcatechol (as described for strain PKO1 [Fishman, A. et al., Biocat. Biotrans., 22:283-289 (2004)]).

PM1 has nine CDSs encoding putative proteins with varying similarity to cyclohexanone monooxygenases (CHMOs) sometimes referred to as Baeyer-Villiger-type MOs (mpeB579, B607, B610, A393, A898, A1038, A1351, A2885, and A2915). Their protein products may play a role in hydroxylation of either alicyclic, aliphatic or aryl ketones to form a corresponding ester, which can easily be hydrolyzed. Alicyclic hydrocarbons represent up to 12% (wt/wt) of total hydrocarbons in petroleum mixtures (American Petroleum Institute). Aryl ketones such as acetophenone can be produced directly from atmospheric breakdown of ethylbenzene (a major petroleum component) or following abiotic conversion of ethylbenzene to ethylphenol (Atkinson, R., Environ. Sci. Technol., 4:65-89 (1995)) and subsequent biological conversion to the ketone. The putative CHMO genes are scattered across the genome and are not present in operons with other genes coding for subsequent metabolism after the MO reaction (i.e., esterases, alcohol and aldehyde dehydrogenases). The CHMOs have a narrow substrate range, possibly explaining the number of different flavoprotein MOs in PM1 with varying levels of similarity with representatives from this class (Table 2); the putative CHMOs in PM1 were more similar to phenylacetone MO (46-67%; Malito, E. et al., Proc. Natl. Acad. Sci. U.S.A., 101:13157-13162 (2004)) than 4-hydroxyacetophenone MO (43-53%; Kalyuzhnaya, M. G. et al., J. Bacteriol., 187:4607-4614 (2005)). In PM1, the nine Baeyer-Villiger MOs have a putative NADP⁺-binding site that is 72-88% similar to the proposed site in a CHMO from Acinetobacter sp. strain NCIMB 9871 (Chen, Y.-C. et al., J. Bacteriol., 170:781-789 (1988)).

An alkane monooxygenase pathway on pPM1 may facilitate PM1's growth on n-alkanes. In addition, alkane monooxygenase (hydroxylase) has been proposed to play a role in cometabolic MTBE hydroxylation since acetylene, an inactivator of short-chain alkane monooxygenase, was shown to inhibit MTBE degradation (Smith, C. A. et al., Appl. Environ. Microbiol., 69:796-804 (2003)). In PM1 the hydroxylase subunit, AlkB (mpeB0606) is 69% and 66% similar to that of Alcanivorax borkumensis AP1 (72) and P. putida PGo1 (contained on the OCT plasmid) (van Beilen, J. B. et al., Microbiol., 147:1621-1630 (2001)) respectively, and contains all 8 of the conserved His residues observed in other integral membrane binuclear-iron hydrocarbon monooxygenases (Hamamura, N. et al., Appl. Environ. Microbiol., 67:4992-4998 (2001)). Also present are two rubredoxin genes (mpeB0603 and mpeB0602), whose products are 76 and 78% similar, respectively, to rubredoxin 3 and 4 in Gordonia sp. strain TF6 (Fujii, T. et al., Biosci. Biotechnol. Biochem., 68:2171-2177 (2004)). The rubredoxin (Rd) coded by mpeB603 is an AlkG1-type Rd, whereas, that coded by mpeB602 is an AlkG2-type Rd, based on the CXXCG motif criteria described by van Beilen et al. (van Beilen, J. B. et al., J. Bacteriol., 184:1722-1732 (2002)); Only AlkG2-type Rds were shown to be functional in electron transfer from the rubredoxin reductase to alkane hydroxylase. In addition, mpeB0601 codes for an ATP-dependent transcriptional regulator 38% similar to AlkS from A. borkumensis SK2 (Hara, A. et al., Environ. Microbiol., 6:191-197 (2004)). Separated from the putative alkS by three hypothetical genes is a rubredoxin reductase, alkT (mpeB0597) whose protein product is 49% similar to that in Gordonia sp. TF6. PM1 does not appear to possess any long-chain alkane (>C₁₃) oxidation pathways such as an alkane dioxygenase (Sakai, Y. et al., Biosci. Biotechnol. Biochem., 58:2128-2130 (1994)), P-450 monooxygenase (Asperger, O. et al., Appl. Microbiol. Biotechnol., 19:3948-4403 (1984)) or two alkane hydroxylase complexes (AlkMa and AlkMb) similar to Acinetobacter sp. strain M-1 (Tani, A. et al., J. Bacteriol., 183:1819-23 (2001)), although PM1's single AlkB is 54% similar to both AlkMa and AlkMb. The gene organization of the alk operon in PM1 is somewhat similar to that of Gordonia sp. TF6 (alkB2G1G2T), except a transposase (mpeB605) and putative esterase (mpeB604) are between alkB and alkG1G2 and as mentioned, a putative alkS and three hypothetical genes (mpeB600-598) are located between alkG1G2 and alkT in PM 1. Homologs to AlkHJKL from P. putida GPo1 coding for aldehyde dehydrogenase, alcohol dehydrogenase, acyl CoA synthetase, and outer membrane protein (van Beilen, J. B. et al., Microbiol., 147:1621-1630 (2001)), respectively, were not present on the plasmid, although the PM1 chromosome contains homologs to AlkH (mpeA2324, 47% similar), AlkJ (mpeA3803, 58% similar), AlkK (mpeA1769, 71% similar) and AlkL (mpeA3010, 49% similar).

In addition, the PM1 chromosome contains a putative propane monooxygenase pathway (mpeA950-953) whose predicted proteins are 41-64% identical to PrmABCD in Gordonia sp. TY-5, coding for the large hydroxylase subunit, the NADH-dependent acceptor oxidoreductase, the small hydroxylase subunit and the coupling protein, respectively. The Prm complex in strain TY-5 was shown to catalyze the subterminal oxidation of propane yielding 2-propanol (Kotani, T. et al., J. Bacteriol., 185:7120-7128 (2003)), while propane oxidation in PM1 is currently under investigation. As for PrmA in Gordonia TY-5, a pair of conserved Glu-X-X-His sequences are present in the putative PrmA of PM1 at residues 138-141 and 237-240. The presence of these sequences is consistent with other monooxygenases in the binuclear-iron oxygenase family including soluble methane monooxygenases (Elango, N. et al., Protein Sci., 6:556-568 (1997); Smith, T. J. et al., Appl. Environ. Microbiol., 68:5265-5273 (2002)) suggesting PrmA in PM1 may catalyze hydroxylation of propane. Like the operon in strain TY-5, a chaperone similar to GroEL was adjacent to the prm cluster in PM1 (mpeA954). Finally, PM1 has homologs to strain TY-5 alcohol dehydrogenases, adh1 (mpeA936) and adh3 (mpeA599) that are 72 and 83% similar, respectively, that may facilitate 2-propanol degradation. The putative monooxygenases in PM1 are summarized in Supplemental Table 3 including methanesulfonate monooxygenase, msmA and alkanesulfonate monooxygenase ssuD, which are part of msmABDCEFGHG and ssuAADCB operons, respectively. PM1 may not utilize methanesulfonate since its msmA lacks the sequence CXH-X₂₆-CXXH unique to methanesulfonate utilizers (Baxter, N. J. et al., Appl. Environ. Microbiol., 68:289-296 (2002)). In general, PM1 possesses several homologous genes with other soil bacteria including Gordonia, Alcinovorax, and Pseudomonas spp. capable of biodegradation of petroleum hydrocarbons as well as xenobiotic and recalcitrant compounds such as phthalates.

MTBE biodegradation. Though MTBE is a recent anthropogenic contaminant (released within the last 15 years), various microorganisms can utilize the compound for carbon and energy under aerobic conditions (François, A. et al., Appl. Environ. Microbiol., 68:2754-2762 (2002); Rohwerder, T. et al., Appl. Environ. Microbiol., 72:4128-4135 (2006); Salanitro, J. P. et al., Appl. Environ. Microbiol., 60:2593-2596 (1994); Steffan, R. J. et al., Appl. Environ. Microbiol., 63:4216-4222 (1997)). M. petroleiphilum PM1 is the best characterized of the few bacterial pure cultures reported to grow on and completely degrade MTBE and its daughter product TBA (Deeb, R. A. et al., Environ. Sci. Technol., 35:312-317 (2001); François, A. et al., Appl. Environ. Microbiol., 68:2754-2762 (2002); Hatzinger, P. B. et al., Appl. Environ. Microbiol., 67:5601-5607 (2001); Salanitro, J. P. et al., Appl. Environ. Microbiol., 60:2593-2596 (1994)). The genetic basis for MTBE and TBA conversion is not known, although different classes of monooxygenases have been proposed to play a role in metabolism or co-metabolism of these compounds (François, A. et al., Appl. Environ. Microbiol., 68:2754-2762 (2002); Hatzinger, P. B. et al., Appl. Environ. Microbiol., 67:5601-5607 (2001); Liu, C. Y. et al., Appl. Environ. Microbiol., 67:2197-2201 (2001); Smith, C. A. et al., Appl. Environ. Microbiol., 69:796-804 (2003); Steffan, R. J. et al., Appl. Environ. Microbiol., 63:4216-4222 (1997)), including P450-monooxygenase and alkane monooxygenase (hydroxylase) systems, the latter shown to play a role in cometabolic degradation of MTBE by P. putida GPo1 (Smith, C. A. and M. R. Hyman, Appl. Environ. Microbiol., 70:4544-4550 (2004)) and possibly also by P. mendocina KR-1 (Smith, C. A. et al., Appl. Environ. Microbiol., 69:7385-7394 (2004)). A known inducer of alkane hydroxylase, dicyclopropylketone, was also shown to induce MTBE conversion to TBA in GPo1 (Smith, C. A. and M. R. Hyman, Appl. Environ. Microbiol., 70:4544-4550 (2004)). As reported above, an alkane MO (AlkB) system was detected in the PM1 genome on the megaplasmid. The AlkB in PM1 is likely involved in MTBE hydroxylation based on similarity to other AlkB proteins in organisms shown to be involved in MTBE degradation. Whereas, the Kₛ values for MTBE in n-alkane grown GPo 1 was reported to be quite high (20-40 mM), the apparent half saturation constant for MTBE by PM1 was 88 μM, which is in the range of Kₛ values for MTBE by butane-degrading bacteria (Liu, C. Y. et al., Appl. Environ. Microbiol., 67:2197-2201 (2001)). Unlike GPo1 and KR-1, PM1 further degrades TBA, ultimately producing CO₂ and biomass. The putative AlkB in PM1 is proposed to only oxidize MTBE and not TBA based on kinetics experiments with MTBE- and TBA-grown cells (Deeb, R. A. et al., Am. Chem. Soc., 219:ENVR 228 (2000)). Two separate enzyme systems were also suggested for MTBE and TBA degradation by Hydrogenophaga flava ENV735 (Hatzinger, P. B. et al., Appl. Environ. Microbiol., 67:5601-5607 (2001)). Because of its potential role in MTBE metabolism, the coding region for AlkB is the focus of current gene knockout studies. Biodegradation of a similar molecule, ethyl-tert butyl ether (ETBE), occurs via a cytochrome P450 pathway in Rhodococcus ruber IFP2001 (Chauvaux, S. et al., J. Bacteriol., 183:6551-6557 (2001)). However, homologs of protein complexes involved in ETBE degradation from R. ruber were not found in PM1; like GPo1 (Smith, C. A. and M. R. Hyman, Appl. Environ. Microbiol., 70:4544-4550 (2004)), PM1 has not been shown to degrade ETBE.

Many pollutant degradation genes are located on bacterial catabolic plasmids. Significantly, the two strains, MG4 and 312 that are capable of MTBE degradation had a nearly identical plasmid to that of PM1 (ca. 99% identical) as determined by comparative genomic sequencing analysis. The MG4 and 312 plasmids showed only 5 or 4 SNPs respectively relative to PM1 (Table 3). MG4 and 312 plasmids also lacked transposase genes (three copies of Tra5 and transposase-8 and one copy of a DDE-domain transposase) that were present on the PM1 plasmid and a 1.2 kb deletion putatively containing an esterase/lipase gene (mpeB604) and a DDE-domain transposase (mpeB605) (Table 3). The promoter and coding region for alkB (mpeB606) did not appear to be affected by this deletion since it is significantly upstream although there was a SNP mapped within alkB of MG4 and 312 resulting in a putative amino acid change. As mentioned, two other PM1 plasmid-encoded genes mpeB541 and mpeB538 code for putative large and small subunits of isobutyryl-coenzyme A (CoA) mutase, respectively. The plasmids of strains MG4 and 312 also contained identical copies of mpeB541 and mpeB538. These gene products were shown to have 99% identical homologs in Ideonella sp. strain L108 predicted to allow conversion of 2-HIBA to 3-hydroxybutyrate in the presence of CoA and ATP (Rohwerder, T. et al., Appl. Environ. Microbiol., 72:4128-4135 (2006)). It is not known whether these mutase genes are contained on a megaplasmid in L108 although horizontal gene transfer is often evoked when such high similarities in gene sequences between bacteria are observed. In addition to alkB, the PM1 plasmid (as well as MG4 and 312 plasmids) contains a gene coding for 3-hydroxybutyryl-CoA dehydrogenase (mpeB0547), putatively involved in conversion of 3-hydroxybutyryl-CoA, a proposed MTBE-metabolite (Rohwerder, T. et al., Appl. Environ. Microbiol., 72:4128-4135 (2006)), to acetoacetyl-CoA.

The role of the megaplasmid in MTBE and TBA degradation was clearly demonstrated by curing experiments. Chemical analysis of MTBE and TBA degradation by the MP0005 parent strain and the MP0007 strain lacking the megaplasmid (as evidenced by PCR analysis and loss of Sm-resistance) showed that only the former was able to degrade MTBE and TBA. This result is consistent with our proposal that key genes involved in both MTBE and TBA degradation are located on the PM1 megaplasmid. Since two different monooxygenases are proposed to be involved in MTBE and TBA degradation (Deeb, R. A. et al., Am. Chem. Soc., 219:ENVR 228 (2000); K. Hristova), at least some of the required protein subunits for conversion of MTBE to TBA and conversion of TBA to the putative 2-methyl-1,2-propanediol are coded on the megaplasmid. In addition, the possible role of selected pPM1 proteins in MTBE/TBA oxidation, based on cDNA microarray results, is currently under investigation by gene knockout methods. It is noteworthy that the PM1 plasmid did not contain predicted proteins with significant homology to those proposed in degradation of 2-methyl-1,2-propanediol by Mycobacterium austroafricanum IFP 2012 (Ferreira, N. L. et al., Microbiol., 152:1361-1374 (2006)), although the putative aldehyde dehydrogenase coded by mpeA1909 on the chromosome was 54% similar to MpdC (hydroxyisobutyraldehyde dehydrogenase) and the alcohol dehydrogenase coded by mpeA945 was 45% similar to MpdB (2-methyl-1,2-propanediol dehydrogenase). While the relevant alcohol and aldehyde dehydrogenases may be encoded on the chromosome, additional plasmid-encoded dehydrogenases may be more plausible and are currently being investigated for their putative role in the MTBE degradation pathway.

Concluding remarks. Prior to sequencing its genome, it was not known that PM1 possessed a 600-kb megaplasmid, much less that the plasmid contained candidate CDSs coding for the MTBE and TBA monooxygenases and enzymes involved in downstream reactions. It is noteworthy that MTBE-degrading strains from diverse locations including a biofilter treating wastewater in Southern California (PM1) and two distinct aquifers in Northern California (MG4 and 312) possess a nearly identical plasmid. The presence of this highly conserved megaplasmid among PM1-like MTBE-degraders, along with its different G+C content, its unique IS complement and the unique phylogenetic distribution of its gene products, together raise interesting questions concerning horizontal gene/plasmid transfer and evolution of pathways via plasmid-mediated mechanisms. With the whole genome sequence, putative aromatic hydrocarbon and alkane degradation pathways were also identified providing a basis to study the complex regulation of fuel hydrocarbon degradation in this novel subsurface bacterium; this is important since substrate interactions are expected to influence the success of bioremediation strategies for gasoline-contaminated sites. In addition to comparative genomics approaches, whole genome microarray and 2-D gel electrophoresis experiments are being conducted to identify genes and proteins unique to MTBE degradation. PM1 can serve as a model for other MTBE-degrading methylotrophs such that the knowledge gained from analysis of its genome, transcriptome and proteome can be applied to PM1-like bacteria. An understanding of the MTBE degradation pathway and its regulation will allow for optimization of MTBE bioremediation and the ability to monitor this unique process in situ using molecular tools.

### EXAMPLE 2

### Microarray Analysis of Genes Involved in MTBE Biodegradation

In this study, the M. petroleiphilum PM1 global transcriptome response in the presence of MTBE and the potential physiological stress brought about by this pollutant was evaluated for the first time. High-density oligonucleotide arrays were employed to explore the genes involved in MTBE biodegradation and to compare gene expression profiles for ethanol and MTBE as growth substrates. Results revealed links between metabolism of MTBE and 1) metabolism of other aromatic compounds present in gasoline mixtures, 2) oxidative stress response, and 3) expression of metal resistance genes.

### Material and Methods

Bacterial strain and genome sequence. Methylibium petroleiphilum strain PM1 is a methylotroph capable of using MTBE as a sole carbon and energy source. The finished sequence of the whole genome of strain PM1 was made available though a collaborative sequencing effort between the University of California, Davis, Lawrence Livermore National Laboratory (LLNL) and the Joint Genome Institute (Walnut Creek, Calif.). At the time this study was initiated, a draft genome sequence of ˜8× coverage consisting of 33 contigs was available. The annotation of this draft sequence, in collaboration with Oak Ridge National Laboratory, resulted in 4006 putative coding sequences (CDSs) that defined the genome. With completion of the genome, the number of CDSs increased to 4479, indicating that, at the time of this expression study, our available sequence information covered nearly 90% of the genome. The complete genome sequence of M. petroleiphilum PM1 is available through National Center for Biotechnology Information (NCBI), GenBank accession numbers NC_{—}008825 for the chromosome and NC_{—}008826 for the plasmid.

Media and growth conditions. M. petroleiphilum PM1 was grown in liquid mineral salts medium, MSM (Tris-HCl, 0.13 M; KH₂PO₄, 0.023 M; K₂HPO₄, 0.025 M; CaCl₂, 0.027 M; NaHCO₃, 0.2 M; MgSO₄, 0.05 M; EDTA, 0.0288 mM; and NH₄Cl, 0.27 M) supplemented with trace elements (CoCl₂, 0.25 μM; CuSO₄, 0.3 μM; FeCl₃, 40 μM; H₃BO₃, 50 μM; MnCl₂, 10 μM; Na₂MoO₄, 0.1 μM; ZnSO₄, 0.8 μM) and either MTBE (250 mg/L) or ethanol (790 mg/L) as the sole carbon source. PM1 is capable of growth on MSM with up to 1000 mg/L MTBE or up to 7.9 g/L ethanol. The dimensionless Henry's constant for MTBE, 0.023, was used to calculate its solution-phase concentration. Cells were grown at 28° C. in 50-ml batch cultures in 150-ml glass bottles with rotary shaking at 150 rpm. At the start of the experiment, bottles were inoculated with ˜5 ml of PM1 culture (grown in the presence of the corresponding carbon source) to achieve an optical density at 595 nm (OD₅₉₅) of ˜0.02. Cells from three biological replicates were harvested at mid-exponential phase after 48 hr of incubation. Final OD₅₉₅ values for the ethanol and MTBE grown cultures were 0.6 and 0.3, respectively. Before RNA extraction, cell densities were adjusted to correspond to 5.9×10⁸ and 2.5×10⁸ colony forming units (CFU)/ml for ethanol and MTBE cultures, respectively. At the time of harvesting approximately 50% of the substrate was utilized.

RNA extraction. Aliquots of 30 mL liquid cultures were treated with RNAprotect to stabilize RNA (QIAGEN, Valencia, Calif., USA) in a ratio 1 part culture to 1.6 parts reagent as outlined by the manufacturer. RNA was subsequently extracted from the cells using a GENTRA Purescript RNA isolation kit (Gentra Systems, Minn., USA) according to the manufacturer's protocol. A DNase treatment step was included after RNA extraction in which DNase I (Roche Inc., Basel, Switzerland) was added to tubes (3 U/10 μg RNA), incubated for 30 min at 37° C., and followed by enzyme inactivation at 95° C. for 5 min. RNA extracts were purified with an RNeasy Mini Kit and RNase-free DNase (QIAGEN) according to the manufacturer's protocols. RNA was finally eluted with RNase-free water and stored at −80° C. until cDNA synthesis. Aliquots were analyzed with a Bioanalyzer (Agilent, Santa Clara, Calif.), which indicated minimal degradation and concentrations ranging from 409 to 620 μg/ml, and A₂₆₀/A₂₈₀ ratios ranging from 1.8 to 2.1.

Preparation of labeled cDNA. cDNA production and labeling were performed by NimbleGen Systems, Inc (Madison, Wis.). After thawing RNA samples on ice, 10 μg total RNA was used to perform cDNA synthesis with random hexamers and SuperScript II reverse transcriptase (Invitrogen, Carlsbad, Calif.). RNase A and H were then used to digest the RNA. The resulting single-stranded cDNA was purified by phenol extraction and precipitated after adding 10 μg glycogen (as carrier), 0.1 volume of ammonium acetate and 2.5 volumes of 100% ethanol. The resulting pellet was dried and suspended in 30 μL water and the cDNA yield was measured by UV/visible spectrophotometry at 260 nm. The cDNA was partially digested with DNAse I (0.2 U) at 37° C. for approx. 13 min, generating 50- to 200-base fragments as observed with a Bioanalyzer (Agilent). The fragmented cDNA was end-labeled with Biotin-N₆-ddATP and terminal deoxynucleotidyl transferase (51 U) during incubation for 2 hr at 37° C. The labeled product was concentrated to 20 μL final volume using a Microcon YM-10 10,000 MWCO filter device (Millipore, Billerica, Mass.) and stored at −20° C. before hybridization.

Microarray design and synthesis: Maskless, light-directed digital micromirror technology (Nuwaysir, E. F. et al., Genome Res. 12:1749-55 (2002)) was used to fabricate high-density 60-mer oligonucleotide microarrays at NimbleGen Systems, Inc. For designing oligonucleotide probes, a database of the gene (CDS) sequences of the M. petroleiphilum PM1 genome (4006 CDSs on Jun. 17, 2004) was created and a file of all possible 60-mers was generated. For each CDS, two to nine 60-base oligonucleotides (probes) were selected based on CDS length such that each probe was at least three mismatches different than all other probes chosen. Probe sets were replicated in triplicate (representing technical replicates) on each chip. A total of 27,704 probes were designed for the genome, and these probes were randomized into a four-to-nine design on the chip (4 spots with same oligonucleotide surrounded by blank spots) to enhance sensitivity. A quality control hybridization using on-chip control oligonucleotides was performed for each array prior to hybridization with labeled cDNA from PM1.

Microarray hybridization. NimbleGen Systems, Inc. Hybriwheel technology was used to perform array hybridization. Briefly, arrays were pre-hybridized at 45° C. in 50 mM MES (4-Morpholineethanesulfonic acid) buffer with 500 mM NaCl, 10 mM EDTA, and 0.005% Tween-20. Herring sperm DNA was added at 0.1 mg/ml to prevent non-specific binding. After 15 min of pre-hybridization, 4 μg of labeled cDNA in hybridization buffer was added to arrays followed by incubation for 16-20 hr at 45° C. Free probe was removed by conducting several wash steps, progressing from less to more stringent conditions. Bound probe was detected with Cy3-labeled streptavidin with signal amplification achieved by adding biotinylated anti-streptavidin goat antibody.

Data Normalization and Gene Expression Analysis: For each experimental condition (MTBE or ethanol growth conditions), there were nine data points for each probe, representing data for three technical replicates of the entire probe set for each of three biological replicates. The arrays were analyzed using an Axon GenePix 4000B Scanner (Molecular Devices Corp., Sunnyvale, Calif.). ImageJ software (http://rsb.info.nih.gov/ij/) was used to rotate images and double their size without interpolation. Features were extracted using GenePix 3.0 software, using a fixed feature size. The log-transformed signal (base 2) was used as the input data for analysis.

Statistical Analysis: Data analysis was performed using the R statistical package and tools available from the Bioconductor project (http://www.bioconductor.org). Data was quantile-normalized (Bolstad, B. M. et al., Bioinformatics 19:185-193 (2003)), and background corrected and summarized using the Robust Multi-array Average (RMA) method (Irizarry, R. A. et al., Nucleic Acids Res. 31:31-34 (2003)). A linear model was fitted for each gene to estimate log-ratios between multiple target RNA samples simultaneously using the LIMMA package (Smyth, G. K. Stat. Appl. Genet. Mol. Biol. 3:Article 3 (2004)). The standard errors of the estimated log-fold changes were moderated using empirical Bayes methods implemented in the LIMMA package, generating a moderated t-statistic. P-values were obtained from this moderated t-statistic, after adjusting for multiple hypothesis testing using Benjamini and Hochberg's method to control the false discovery rate (Dudoit, S. et al., Stat. Sci. 18:71-103 (2003)). Genes with p-values <0.05 and a fold change ≧2 were chosen to be significantly upregulated or downregulated (actual p-values for this group were <0.001). Annotation of the significantly differentially expressed genes was derived from the Cluster of Orthologous Genes (COG) annotation for the PM1 genome. Studies of the gene ortholog neighborhood were done using the Integrated Microbial Genome (IMG) database (Joint Genome Institute).

Reverse transcription, quantitative PCR analysis. Confirmation of transcript levels for modulated genes was performed by reverse transcription quantitative PCR (RT-qPCR) analysis of RNA samples extracted from ethanol- and MTBE-grown cultures. Since sufficient RNA was not available from extracts used in microarray experiments, separate cultures were grown under the same conditions and extracted for RNA using the same method as described above. Total RNA (˜300-1500 ng) was converted to cDNA using random hexamers and MultiScribe reverse transcriptase (Applied Biosystems, Foster City, Calif.). The resulting cDNA was amplified using an IQ SYBR Green RT-PCR kit (Bio-Rad, Hercules, Calif.) with gene-specific primers for eighteen different CDSs on a MyIQ single-color real-time PCR cycler (Bio-Rad). Calibration curves were performed with genomic DNA serially diluted over a range of five to six orders of magnitude. The PCR conditions were optimized as follows: 95° C. for 5 minutes; 40 cycles of 94° C. for 15 seconds, 58° C. for 30 seconds, and 72° C. for 30 seconds. The primers are listed in Table S1 in the supplemental material. The RNA transcript amount was normalized to the total amount of starting RNA quantified using a Bioanalyzer.

Sequence analyses and generation of phylogenetic trees. Homologs of M. petroleiphilum PM1 translated CDSs were identified using BLASTP searches against the non-redundant (NR) GenBank database from NCBI. Sequences were aligned and alignments were refined using ClustalX version 1.8 (Thompson, J. D. et al., Nucleic Acids Res. 25:4876-4882 (1997)) along with manual adjustments. The protdist program and the neighbor program of the Phylip package were used to generate phylogenetic trees. MacVector software (Accelrys, San Diego, Calif.) version 9.0 was also used to generate the phylogenetic trees for MdpA and MdpJ using Neighbor Joining/Best Tree methods with systematic tie-breaking and gaps distributed proportionately.

Microarray data accession number. Microarray data have been deposited in the gene Expression Omnibus database (http://www.ncbi.nlm.nih.gov/) under accession number xxxxx.

### Results and Discussion

Global summary of differential expression of PM1 genes during growth on MTBE. In response to growth on MTBE, 1255 genes of the 3941 genes represented on the arrays were differentially expressed, with 440 genes more than twofold upregulated and 815 genes more than twofold downregulated in comparison to growth on ethanol (Tables S2 and S3 in the supplemental material). Importantly, our analyses identified a large number (172 of 440 upregulated and 119 of 815 downregulated) of genes with unknown functions whose expression was altered during exposure to MTBE. The upregulated genes are of interest for subsequent functional analyses as they may be involved in MTBE catabolism. Differentially expressed genes were sorted according to functional categories (Tables 1, 2, 3, and Tables S4 and S5 in the supplemental material).

Transcript levels from the 34 ribosomal protein genes and several genes whose products are involved in translation were lower (2-25 fold downregulated; Table 1) during PM1 growth on MTBE than on ethanol, which most likely reflects a lower growth rate and presumably lower ribosome content. This is in agreement with the observed difference in the doubling time of PM1 cells growing on ethanol, t_{1/2}=6.1 h, vs. cells growing on MTBE, t_{1/2}=15 h. Several key components of the aerobic electron transport chain were also downregulated. Expression levels of various components of the electron transport chain, such as NADH dehydrogenases (Complex I; mpeA1403-1416), ubiquinol-cytochrome c reductase (mpeA0849), cytochrome c oxidases (mpeA2475, mpeA3177, mpeA3179-81, mpeA0432) and several other cytochromes (Table 1) were significantly lower, supporting the notion that MTBE is a less energetically favorable compound and/or more recalcitrant than ethanol. It seems likely that simultaneous downregulation of genes encoding NADH dehydrogenase and NADH:ubiquinone oxidoreductase (mpeA1411) reflects the suppression of the TCA cycle. Several enzymes involved in TCA energy metabolism were also downregulated on MTBE (Table 1).

Additionally, expression of genes involved in biosynthesis of the cell wall, exopolysaccharide and lipopolysaccharide, as well as the capsule polysaccharide export system were downregulated on MTBE in comparison to the ethanol treatment (See Table S4 in the supplemental material). A similar trend in the expression of these genes was observed when Rhodobacter sphaeroides cells were exposed to H₂O₂ (Zeller, T. et al., J. Bacteriol. 187:7232-7242 (2005)), an oxidative agent that could increase the cell wall permeability. Our transcriptional data indicate that MTBE exposure induces a membrane response since several membrane proteins were upregulated 2.0- to 52-fold when PM1 cells were exposed to MTBE (See Table S4 in the supplemental material) including the RND family (mpeA1627, mpeA1649, mpeA2358, mpeA2964). If MTBE acts as an organic solvent, it may accumulate in the cytoplasmic membrane disturbing its structure and function. Under these conditions, the membrane could lose its integrity and increase its permeability to protons, ions, metabolites, lipids and proteins (Segura, A. et al., Environ. Microbiol. 1:191-198 (1999)). Effective removal of the solvent from the cytoplasm or the membranes is one of the key protective mechanisms (Llamas, M. A. et al., J. Bacteriol. 185:4707-4716 (2003)). M. petroleiphilum PM1 is tolerant of concentrations of MTBE up to 5,000 mg/L on tryptic soy broth media. The relationship between PM1's ability to grow on MTBE and the observed changes in the outer and cytoplasmic membranes and cell wall requires further investigation.

Ethanol oxidation in PM1—the Quinoprotein Ethanol Dehydrogenase (QEDH) regulon. While the primary focus of this study was the elucidation of genes involved in MTBE degradation, a converse analysis of the data provided information for genes upregulated in response to ethanol and provided validation of the microarray dataset. In response to growth on ethanol, the most significantly upregulated gene cluster (2.7- to 79.6-fold) in PM1 is the QEDH cluster (Table 2) compared with growth on MTBE. The QEDH regulon extends from mpeA0473 to mpeA0481 and includes the quinoprotein ethanol dehydrogenase genes, exaA1 (mpeA0476) and exaA2 (mpeA0473), and two copies of the cytochrome c-550 precursor gene, exaB1 (mpeA0480) and exaB2 (mpeA0474). Quinoprotein alcohol dehydrogenases are a family of proteins found in methylotrophic or autotrophic bacteria that use pyrroloquinoline quinone as their prosthetic group and contain a C-terminal cytochrome C domain (Hefti, M. H. et al., Eur. J. Biochem 271:1198-1208 (2004)). A two-component regulatory system consisting of a sensor histidine kinase gene exaD (mpeA0477) and a response regulator gene exaE (MpeA478) is present in the PM 1 operon, like that of the ethanol oxidation regulon in Pseudomonas aeruginosa ATCC 17933 (Gliese, N. et al., Microbiol. 150:1851-1857 (2004)). The QEDH regulon also contains a gene with unknown function (mpeA475) that was highly upregulated in cells grown on ethanol (45-fold).

The two putative quinoprotein ethanol dehydrogenases, MpeA0476 and MpeA0473, share a 52% identity. MpeA0476 showed a significantly higher identity to the ExaA from Pseudomonas aeruginosa (70%), as compared to MpeA0473 (53%). In addition, the expression level of mpeA0476 was much higher than that of mpeA0473 (˜80-fold vs. 3-fold). The role of the mpeA0473 dehydrogenase gene has not been clearly elucidated, however an exaA knockout in P. aeruginosa did not eliminate ethanol oxidation suggesting metabolic redundancy and a role for a second dehydrogenase (Vrionis, H. A. et al., Appl. Microbiol. Biotechnol. 58:469-475 (2002)). Based on the ethanol degradation pathway in E. coli, it is likely that mpeA0476 codes for an alcohol dehydrogenase that converts ethanol to acetaldehyde. This product is likely converted to acetyl-CoA (Keseler, I. M. et al., Nucleic Acids Res. 33:D334-337 (2005)) by the second NADH dehydrogenase (acetaldehyde dehydrogenase, ExaC, MpeA0599) which shows 71% identity to ExaC (from the ExaABC cluster in P. aeruginosa) and is suspected to play a role in ethanol oxidation (Schobert, M. et al., Microbiol. 145:471-481 (1999)). In addition, the gene mpeA0599 is upregulated 11-fold on ethanol. A comparison of the exaB1 protein product (MpeA0480) with its counterpart in P. aeruginosa showed 50% identity, in contrast to that of exaB2 (MpeA0474), which was only 33% identical. It is not known if one or both putative cytochromes c-550 function in electron transfer during ethanol degradation.

Evaluation of the expression ratios by quantitive RT-PCR. RT-qPCR was employed to confirm the trends observed in the expression data. Eighteen genes, chosen based on genomic location and differential expression, were compared using the two techniques. In general, the RT-qPCR results expressed as log difference between MTBE- and ethanol-grown cells showed the same trends as the log differences for the same treatments for the microarray; the data were well correlated (r²˜0.95). For some of the CDSs, including mpeB0606 and B0559, the RT-qPCR log difference was considerably greater than the microarray fold-difference for MTBE versus ethanol (1.5-fold and 0.7-fold for microarray analysis and 500- and 790-fold for RT-qPCR analysis for mpeB0606 and B0559, respectively), which caused the slope to deviate from 1. Attempts were made to reproduce growth conditions since the same extracts used in the microarray analysis were not available for RT-qPCR analysis, thus it is likely that slight variations in culture conditions were present. Because of these slight variations in culturing, the variability between analyses is likely higher, but the trends seen in the microarray analysis are consistent with those observed with RT-qPCR analysis. The smaller fold differences observed with the microarray data may be caused in part by data normalization, which tends to compress the microarray data.

PM1 biodegradation capacity for pollutants. M. petroleiphilum PM1 grows on phenol and two distinct clusters of dimethylphenol (dmp)-like genes are present on the chromosome (mpeA2265-67, 2272-86; mpeA3305-13, 3321-25). Compared to growth on ethanol, in MTBE-grown cells, significant upregulation of structural genes in the Dmp pathway (P<0.05) was observed for dmp operon I, but not in dmp operon II, except for dmpH. Genetic analysis of the dmp operon II suggested it was not functional since it lacks DmpP (phenol hydroxylase reductase) and DmpC (2-hydroxymuconic semialdehyde dehydrogenase). Additionally, upregulation of the toluene degradation pathway via toluene-monooxygenase was observed when cells were grown on MTBE only. In this case structural genes from both operons were upregulated (Table 3).

Of interest was the differential expression of the regulators of the tbu (toluene) and dmp (phenol) degradation operons. Both tbu operons of PM1 have a two component sensor-regulator gene pair located immediately downstream of the operon. These regulators are divergently expressed but showed less than 2-fold expression increases in the presence of MTBE. Only dmp operon I showed significant up-regulation (1.98-3.06 fold). Included among the upregulated genes was a LysR family type regulator encoded by mpeA2279, which is most similar to aphT of Commamonas testosteroni (Arai, H. et al., Microbiol. 146:1707-1715 (2000)). AphT is related to regulators of pathways for ortho-cleavage of catechol or chlorinated catechols. The F is family regulator gene mpeA2286, closely related to the phenol regulator gene dmpR (GenBank accession no. CAA48174) did not show differential expression under our test conditions.

Several genes coding for enzymes involved in degradation of aromatic compounds, including phenylacetic acid degradation proteins (mpeA0987, mpeA0989), phenylpropionate dioxygenase (mpeA1001), and 2-polyprenylphenol hydroxylase (mpeA0819) were also upregulated in cells grown on MTBE.

M. petroleiphilum PM1 contains an alkane monooxygenase pathway on its plasmid and a propane monooxygenase pathway on its chromosome which may facilitate its growth on n-alkanes. The alkane monooxygenase is discussed later in the context of the MTBE degradation pathway. The propane monooxygenase (pmo) reductase (mpeA0951) was upregulated approximately 4.4-fold in MTBE-grown relative to ethanol-grown cells. It is not currently known whether PM1 can grow on propane or whether the Pmo pathway is functional.

The PM1 genome has nine CDSs with similarity to cyclohexanone monooxygenases (CHMOs) (33). In MTBE grown cells, there was greater than 2-fold upregulation of three CHMO genes (mpeB0610, mpeA0393, mpeA1351) and downregulation of one CHMO gene (mpeA0607) (Table 3).

Discovery of the genes involved in the aerobic MTBE degradation by M. petroleiphilum PM1. Using two independent approaches, comparative genomic hybridization (CGH) and plasmid curing, we previously demonstrated that MTBE/TBA degradation genes are located on the PM1 plasmid (Kane, S. R. et al., J. Bacteriol. 189:1931-1945 (2007)). In this study, by comparing the whole transcriptome response of MTBE-grown and ethanol-grown cells, a large MTBE degradation regulon consisting of four major gene clusters was identified on the plasmid. Genes in these clusters were designated mdp for MTBE degradation pathway.

The relative gene expression levels in three of these clusters ranged from 2.0- to 12.4-fold. Within two clusters mpeB0555-51 and mpeB0558-62 (upregulated 3.3- to 12-fold on MTBE), a putative iron-sulfur oxidoreductase belonging to the family of ferredoxin reductases, a hydroxylase similar to phthalate dioxygenase, and two dehydrogenase genes mdpE (mpeB0558) and mdpH (mpeB0561) were identified. Additionally, the nearby gene mdpA (mpeB0606), 69% and 66% similar to alkane monooxygenase, AlkB of Alcanivorax borkumensis AP1 (Smits, T. H. M. et al., J. Bacteriol. 184:1733-1742 (2002)) and Pseudomonas putida PGo1 (carried on the OCT plasmid) (van Beilen, J. B. et al., Microbiol. 147:1621-1630 (2001)) respectively (Kane, S. R. et al., J. Bacteriol. 189:1931-1945 (2007)), was 1.5-fold upregulated on MTBE-grown cells. Additionally, mdpA was 4.7-fold upregulated on ethanol-grown cells exposed to MTBE for four hours relative to ethanol-grown cells (data not shown). Gene mdpA was also highly upregulated on MTBE, 500-fold relative to ethanol based on RT-qPCR analysis. This suggests that mdpA may be expressed early in response to the presence of MTBE and is consistent with our proposal that MdpA is a MTBE monooxygenase responsible for the initial oxidation reaction of MTBE to tert-butoxy methanol. This hypothesis is in agreement with previous physiology studies of strain PM1 showing that two different oxygen-dependant enzymes were involved in MTBE and TBA oxidation (Deeb, R. A. et al., Abstr. Pap. Am. Chem. Soc. 219:ENVR 228 (2000)), (K. Hristova, unpublished data) and with the fact that an mdpA insertion mutant could not degrade MTBE (R. Schmidt, manuscript in preparation).

We hypothesize that gene mdpE (mpeB0558), 12-fold upregulated on MTBE, coding for a dehydrogenase may be involved in the production of tert-butyl formate (THF). The conserved motif (²³⁰-GQHKGSA-²³⁶) and the conserved residue E³¹⁹ of MdpE clearly identify it as a member of a recently described family of (S)-2-hydroxyacid dehydrogenases that bind NADP/NADPH as cofactors in a novel, non-Rossman fold (Irimia, A. et al., EMBO J. 23:1234-1244 (2004); Muramatsu, H. et al., J. Biosci. Bioeng. 99:541-547 (2005); Muramatsu, H. et al., J. Biol. Chem. 280:5329-5335 (2005)). With one exception, these functionally diverse enzymes act on 2-oxo or 2-hydroxy acids (Muramatsu, H. et al., J. Biosci. Bioeng. 99:541-547 (2005); Muramatsu, H. et al., J. Biol. Chem. 280:5329-5335 (2005); Yew, W. S. et al., J. Bacteriol. 184:302-306 (2002)). Sequence alignment and phylogenetic analysis of MdpE with proteins belonging to the seven proposed clades of (S)-2-hydroxyacid dehydrogenases (Muramatsu, H. et al., J Biosci. Bioeng. 99:541-547 (2005); Muramatsu, H. et al., J. Biol. Chem. 280:5329-5335 (2005)) suggests that this enzyme is deeply branching. Therefore it is not possible to assign this enzyme into any of the described groups, as it may represent a separate clade. However, based on the functionality of the MdpE enzyme and its 12-fold increase in expression on MTBE, we propose this enzyme to be the dehydrogenase required for complete conversion of MTBE to the intermediate THF.

It has been demonstrated that the hydrolysis of THF to TBA occurs spontaneously and rapidly under low pH conditions (Church, C. D. Environ. Toxicol. Chem. 18:2789-2796 (1999); Smith, C. A. et al., Appl. Environ. Microbiol. 69:796-804 (2003)). However, on the basis of growth in a buffered mineral medium used in this study, as well as physiology studies in other organisms (Smith, C. A. et al., Appl. Environ. Microbiol. 69:796-804 (2003)), it seems most probable that THF hydrolysis in PM1 is an esterase-catalyzed process. A gene for an esterase (mpeB0604) is located downstream of mdpA on the megaplasmid, but our analyses provide evidence that preclude its involvement in THF hydrolysis. This esterase gene was not significantly differentially expressed on MTBE (it was downregulated 1.2 fold), which may be the result of interruption by an ISmp1 element. In addition, an mpeB0604 homolog is lacking in PM1-like MTBE-degrading environmental isolates that also lack the ISmp1 element (Kane, S. R. et al., J. Bacteriol. 189:1931-1945 (2007)), suggesting the involvement of another esterase.

No other prospective esterases were found on the megaplasmid, however a 5.2-fold upregulated gene for a possible THF esterase was found on the main chromosome (mpeA2443). MpeA2443 belongs to the hormone-sensitive lipase family. The bacterial members of this family are known to act on short chain (4-8 C) carboxylic esters, but their physiological function is largely unknown (Haruki, M. et al., FEBS Lett. 454:262-266 (1999)). MpeA2443 is most closely related (53% identity) to a putative esterase from Rhodococcus sp. RHA1 (GenBank accession no. YP_{—}706618), and more distantly related to acetyl esterases such as Aes of E. coli (Haruki, M. et al., FEBS Lett. 454:262-266 (1999)). An alignment with Aes identified residues Gly¹⁵⁴, Asp¹⁵⁵, Ser¹⁵⁵, and Gly¹⁵⁷ of MpeA2443 as components of the conserved G-D/E-S-A-G motif, and Ser¹⁵⁶, Asp²⁵¹ and His²⁸¹ as the active site residues (Haruki, M. et al., FEBS Lett. 454:262-266 (1999)). Interestingly, it appears that an ISmp4 element (one of 12 on the chromosome; all >99% identical) has recently inserted itself at amino acid position 283 of MpeA2443 (292 aa). Further physiology and genetic studies are required to clarify whether MpeA2443 functions as an esterase, whether it is responsible for THF hydrolysis in PM1, and whether the insertion sequence has had any effect on its function. The gene immediately upstream, mpeA2442, codes for a carboxylesterase 29% identical to BioH of E. coli (Sanishvili, R. et al., J. Biol. Chem. 278:26039-26045 (2003)), but since this gene was upregulated only 1.7 fold (relative to 5.2-fold for mpeA2443) it may not play a role in the MTBE pathway.

The monooxygenase enzyme, alkane hydroxylase alkB, was suggested to be responsible for TBA oxidation in M. austroafricanum strains (Ferreira, N. L. et al., Microbiol. 152:1361-1374 (2006)), as well as in co-metabolic oxidation of MTBE and TBA in M. vaccae JOB5 (Smith, C. A. et al., Appl. Environ. Microbiol. 69:796-804 (2003)). However, based on the microarray analyses, sequence comparisons and protein homology modeling, we propose that a new Rieske non-heme iron subunit (mdpJ; 11.7-fold upregulated on MTBE) of a multi-component enzyme system, and an associated Fe—S reductase (mdpK; 4.3-fold upregulated on MTBE), are involved in TBA oxidation in PM1. Interestingly, immediately upstream, and even possibly contributing to the promoter of mdpJ, lies a unique insertion sequence 66% identical to the ISmp4 family of IS elements.

A more detailed sequence analysis of MdpJ was performed due to its high up-regulation (11.7-fold) on MTBE. The analysis identified a N-terminal Rieske-type [2Fe-2S] domain (C⁸⁵—X—H—X16-C—X2-H¹⁰⁷) and a conserved C-terminal mononuclear, non-heme iron-binding motif (D/E¹⁹⁰-X3-D-X2-H—X4-H²⁰²) typical of Rieske non-heme iron dioxygenases. This class of enzymes uses molecular oxygen, adding both atoms of O₂ to the aromatic ring of the substrate, including aromatic and polycyclic aromatic hydrocarbons (PAH), chlorinated aromatic, nitroaromatic, aminoaromatic, and heterocyclic aromatic compounds. Enzymes in this family are also involved in benzylic and methyl group hydroxylation, desaturation, sulfoxidation and dealkylation reactions (Parales, R. E. et al., Aromatic ring hydroxylating dioxygenases, p. 287-340. In J.-L. Ramos and R. C. Levesque (ed.), Pseudomonas, Volume 4. Springer, Netherlands (2006)). A phylogenetic comparison of the Rieske domain of MdpJ with a number of aromatic ring hydroxylating dioxygenases and several hypothetical proteins showed it belongs to the phthalate group (group I) of dioxygenases as described by Parales and Resnick (Parales, R. E. et al., Aromatic ring hydroxylating dioxygenases, p. 287-340. In J.-L. Ramos and R. C. Levesque (ed.), Pseudomonas, Volume 4. Springer, Netherlands (2006)). This grouping was of particular interest since some enzymes of the phthalate family function as monooxygenases and not dioxygenases with their native substrates (Parales, R. E. et al., Aromatic ring hydroxylating dioxygenases, p. 287-340. In J.-L. Ramos and R. C. Levesque (ed.), Pseudomonas, Volume 4. Springer, Netherlands (2006)). There are also examples of dioxygenases that function as monooxygenases with substrates other than the ‘native’ substrate. Those best studied are toluene and naphthalene dioxygenases and the change in functionality in each case is probably a result of positioning of the compound in the active site (e.g. (Resnick, S. M. et al., J. Indust. Microbiol. Biotechnol. 17:438-457 (1996); Robertson, J. B. et al., Appl. Environ. Microbiol. 58:2643-2648 (1992))). In addition, substitution of a smaller residue at the active site of the tetrachlorobenzene dioxygenase TecA from Ralstonia sp. PS12 (F366L) resulted in a shift from dioxygenation of the aromatic ring to monooxygenation of the methyl group of mono- and dichlorotoluenes (Pollmann, K. et al., Microbiol. 149:903-913 (2003)), thus indicating that single amino acid substitutions are sufficient for dioxygenase-to-monooxygenase switching. Unfortunately, lack of active site analysis data for enzymes closely related to MdpJ precludes detailed predictions for MdpJ substrate specificity. However, together with the high expression value of 11.7-fold in MTBE-grown cells, MdpJ could be envisioned to carry out the hydroxylation of TBA to 2-methyl-2-hydroxy-1-propanol in M. petroleiphilum PM1.

The protein product of the gene immediately downstream of mdpJ, mdpK, shares 39% identity with PobB, a reductase component of phenoxybenzoate dioxygenase. In addition, MdpK contains domains typically conserved in class IA oxygenase ferredoxin reductases: a flavin mononucleotide (FMN) isoalloxazine-binding domain (⁶¹RxYSL⁶⁵), an NAD ribose-binding domain (¹²⁵GGIGITP¹³¹) and a [2Fe-2S] ferredoxin binding domain (²⁸⁸Cx4Cx2Cx29C³²⁴) situated at the C-terminus (van der Geize, R. et al., Microbiol. 148:3285-3292 (2002)). The MdpK protein therefore most likely represents the ferredoxin reductase component of the predicted TBA hydroxylase. The presence of a specific and unique TBA oxidation enzyme system in PM1, responsible for the oxidation of TBA to 2-methyl-2-hydroxy-1-propanol, may help explain the unique capability of PM1 to efficiently degrade TBA without substantial accumulation of the intermediate.

The conversion of 2-methyl-2-hydroxy-1-propanol (MHP) to 2-hydroxyisobutyrate (HIBA) was recently hypothesized to be a two-step process involving alcohol dehydrogenase (MpdB) and aldehyde dehydrogenase (MpdC) in M. austroafricanum IFP 2012 (Ferreira, N. L. et al., Microbiol. 152:1361-1374 (2006)). A BLAST search with the M. austroafricanum IFP 2012 predicted amino acid sequence of the mpd cluster genes mpdB and mpdC retrieved from the NCBI nr database against the PM1 whole genome sequence database showed the highest similarities to MpeA0945 (33% identity) and MpeA1909 (40% identity), respectively (Kane, S. R. et al., J. Bacteriol. 189:1931-1945 (2007)). Both genes are located on the chromosome and the expression of mpeA0945 and mpeA1909 is neutral and decreased 1.7-fold, respectively, on MTBE in comparison to ethanol, making their involvement in MTBE degradation unlikely. Based on microarray data, we propose that in PM1 a putative plasmid-encoded dehydrogenase mdpH (mpeB0561), upregulated 4.6-fold on MTBE, acts as the MHP dehydrogenase in PM1. Interestingly, this gene shows 32% identity and 52% similarity to human 3-HIBA dehydrogenase (HIBADH).

The identity of the hydroxyisobutyraldehyde (HIBAL) dehydrogenase is less clear. In total, there are 11 genes belonging to the aldehyde dehydrogenase superfamily in the PM1 genome, but none showed significant upregulation when grown on MTBE. Based on predicted function, gene arrangement and 1.4-fold upregulation, we propose mpeA0361 as the most likely candidate gene for HIBAL dehydrogenase in PM1. The chromosome-encoded MpeA0361 shows 33% identity and 53% similarity to MpdC of M. austroafricanum IFP2012. More significantly, the peptide shows 58% identity and 74% similarity to AldA of E. coli, an enzyme active on a number of small α-hydroxyaldehyde substrates including lactaldehyde, glycolaldehyde and methylglyoxal (Baldoma, L. et al., J. Biol. Chem. 262:13991-13996 (1987); Di Costanzo, L. et al., J. Mol. Biol. 366:481-493 (2007); Hidalgo, E. et al., J. Bacteriol. 173:6118-6123 (1991)). The downstream gene (mpeA0360) codes for a predicted L-lactate dehydrogenase, therefore confirming the predicted function of MpeA0361 as a lactaldehyde dehydrogenase, and a possible HIBAL dehydrogenase based on substrate similarity. A transposition hotspot located 13-kb upstream of mpeA0361 includes 3 identical and parallel ISmp2 elements (encoding MpeA0375 A0381, A0384 transposases) and a single ISmp1 element (MpeA0382 transposase).

Based on comparative sequence analysis, the products of mdpP (mpeB0539), mdpX (mpeB0547), and mdpO/R (mpeB05381541) were annotated as 2-HIBA CoA-ligase, 3-hydroxybutyryl-CoA dehydrogenase and a two-component HIBA mutase, respectively (Kane, S. R. et al., J. Bacteriol. 189:1931-1945 (2007)). These genes were expressed 1.4-, 1.6- and 1.2/2.7-fold, respectively, on MTBE-grown relative to ethanol-grown cells. The function and the expression of MdpX and MdpO/R are in agreement with a recently proposed pathway of HIBA degradation by a cobalamin-dependent mutase (Rohwerder, T. et al., Appl. Environ. Microbiol. 72:4128-4135 (2006)). In addition we propose MdpP as the 2-HIBA-CoA ligase. A neighboring gene, mpeB0543 is a putative ATP-binding cobalamin adenosyl transferase.

The product of 3-hydroxybutyryl-CoA dehydrogenase, acetoacetyl-CoA, is potentially converted by a predicted acetyl-CoA acetyltransferase (MpeA3367) to two acetyl-CoA molecules which can feed into the tricarboxylic acid (TCA) cycle. MpeA3367, which shows 45% identity to the acetyl-CoA acetyltransferase Th1A of Clostridium acetylbutylicum, is upregulated 1.6-fold on MTBE. This enzyme can also catalyze the reverse reaction as the first step in synthesis of polyhydroxybutyrate (PHB), which may be used as a carbon and energy reserve when nitrogen and/or phosphorus are limiting.

The microarray expression data also revealed significant upregulation of genes with unknown function belonging to cluster mpeB0532-35. These genes do not show high similarity to any known proteins based on simple BLASTP searches. At this point based on the gene expression data alone, we cannot formulate hypotheses for the role of the mpeB532-35-cluster, except to note that they are highly expressed in MTBE-grown cells, and could be involved in degradation of MTBE or aromatic compounds in M. petroleiphilum PM1.

MTBE pathway—gene arrangement and mobilization. Genes specifying the biodegradation of recalcitrant compounds are usually clustered on the same genomic locus, although degradative genes can also be widely separated. Examples of the latter arrangement include the dioxin/dibenzofuran pathway of Sphingomonas sp. strain RW1, whose degradative genes were found to be scattered around the chromosome (Armengaud, J. et al., J. Bacteriol. 181:3452-3461 (1999)), the chromosomally-encoded naphthalene conversion to salicylate and plasmid-encoded salicylate degradation in P. putida PMD-1 (Zuniga, M. C. et al., J. Bacteriol. 147:836-843 (1981)), and the m-toluate degradation genes from pWWO that are integrated in the chromosome of Pseudomonas sp. strain B13-WR211. In PM1, the majority of the MTBE pathway genes appear to be localized to a main cluster (mpeB0538-mpeB0562), but several genes are found on the plasmid outside of this locus, and at least two are predicted to be on the chromosome.

Often, degradative genes or gene clusters are flanked by insertion sequences forming degradative transposons. This allows for the shuttling of catabolic genes and entire gene clusters between different replicons (Top, E. M. et al., Curr. Opin. Biotechnol. 14:262-269 (2003)). The PM1 genome has a number of complex repetitive elements, including eight families of insertion sequences (ISmp1-8) and two large genomic segments that appear to have undergone recent duplications, including the plasmid-based 29-kb phosphonate transport/cobalamin biosynthesis island found twice in tandem and flanked by ISmp8 elements, and a 40-kb duplication found on both the chromosome and the plasmid, where it appears to have integrated and interrupted the deoxycytidine deaminase gene mpeB0168/202 (Kane, S. R. et al., J. Bacteriol. 189:1931-1945 (2007)).

In addition to the predicted contribution of IS elements in expression (e.g. ISmp1 interrupts the mpeB0604 esterase, and a divergent ISmp-4-like IS element may even contribute to the observed expression and regulation of Rieske non-heme iron dioxygenase mdpJ) and coding sequence (e.g. ISmp4 may contribute some sequence to the 3′ end of the mpeA2443 esterase gene) of key MTBE degradative enzymes, the presence of many IS elements in the vicinity of the main MTBE pathway gene cluster may also enable mobilization (as a portable “functional cassette”) and confer selective advantage if retained. The ISmp8 element is restricted to only one strand on the plasmid (5 copies), and thus has the potential for deletions or to transpose larger segments as a transposon. In fact, the majority of the MTBE gene cluster is flanked by two ISmp8 (transposases mpeB0489 and mpeB0570) that are 79 kb apart. Similarly, ISmp7 copies are found within (mpeB0549/50, mpeB0572/1, mpeB0586/7) and flanking (mpeB0004/5, mpeB0070/1) the MTBE gene cluster and could be involved in gene rearrangement, deletion and mobilization. Interestingly, ISmp1 (3 copies), ISmp7 (7 copies) and ISmp4 (12 copies) were among the 5% of genes showing highest expression on both ethanol and MTBE. This high expression was observed even though multiple copies of each IS sequence (except for ISmp1) were present in the microarray.

The G+C content of the mpeA0375-384 IS island is 65.0%, compared with an average of 69.2% for the chromosome and 66.0% for the plasmid, and encodes several hypothetical proteins and lies within a region rich in hypothetical genes. While these represent the only 3 ISmp2 in the genome, the ISmp1 in this region is identical to the one located on the plasmid (mpeB0605) which we predict disrupts the esterase mpeB0604 and lies downstream of mdpA (mpeB0606). A third copy (94% identical) of ISmp1 lies between, or possibly interrupts two hypothetical chromosomal genes (mpeA2597, mpeA2599), which are flanked by inverted copies of ISmp4. This latter IS island has an G+C content of 66.4% and also lies within a number of hypothetical genes which themselves are flanked by the PQQ biosynthesis operon on one side (mpeA3829, mpeA2585-8), and the methanol utilization two-component signal transduction system (mpeA2603-4).

Several of the PM1 IS elements are also similar to ones found associated with catabolic transposons in other environmental bacteria. The ISmp1 transposases shows 84% identity to transposase from a catabolic transposon carrying tfd genes for 2,4-dichlorophenoxyacetic acid degradation on pEST4011 of Achromobacter denitrificans (Vedler, E. et al., J. Bacteriol. 186:7161-7174 (2004)). The unique MpeB0528 and Mpe0529 transposases (part of a predicted composite IS element consisting of genes mpeB0527-9 and located just upstream of the main MTBE gene cluster) show 68% and 64% identity, respectively, to a transposase associated with catechol 1,2 dioxygenase in Burkholderia sp. TH2 (Suzuki, K. et al., J. Bacteriol. 184:5714-5722 (2002)). Finally, a transposase associated with the p-toluenesulfonate degradation transposon of pTSA in Comamonas testosteroni T-2 shows 71% identity to the ISmp8 transposase. The possible involvement of IS elements in mobilization of MTBE genes onto or from the plasmid, as well as in disrupting functions and/or regulation of expression is intriguing from the standpoint of genome and metabolic pathway evolution. Further research is required in order to answer these and other pertinent questions.

Environmental stress response genes. In natural environments, bacteria have to cope with oxidative stress caused by reactive oxygen species (ROS) produced by exposure to metals, redox-active chemicals, or radiation (Velazquez, F. et al., Environ. Microbiol. 8:591-602 (2006); Zeller, T. et al., J. Bacteriol. 187:7232-7242 (2005)). The analysis of the M. petroleiphilum PM1 transcriptome reveals expression of enzymes directly involved in ROS detoxification, protein repair, and DNA repair when PM1 cells were grown on MTBE. Expression data indicate that transcription of ohr (mpeA0058), coding for an organic hydroperoxide resistant protein, is significantly increased when PM1 is grown on MTBE. While this could represent a general stress response it is also possible that an organic peroxide compound could be produced during the oxidation of MTBE. Genes for the catalase (KatE) subunits (mpeA3740 and mpeA1580) involved in bacterial oxidative stress response to H₂O₂ are also upregulated on MTBE. Other genes upregulated in MTBE-grown cells are those coding for glutathione S-transferase (mpeA1566, mpeA0906, mpeA1783; Table 3), which detoxifies xenobiotic compounds, heavy metals or products of oxidative stress by covalently linking glutathione to hydrophobic substrates (Habig, W. H. et al., J. Biol. Chem. 249:7130-9 (1974); Vuilleumier, S. et al., Appl. Microbiol. Biotechnol. 58:138-146 (2002)) in bacteria and humans (Habig, W. H. et al., Methods Enzymol. 77:218-31 (1981)). Glutathione S-transferase genes have also been found in bacterial operons and gene clusters involved in the degradation of aromatic compounds (Vuilleumier, S. et al., Appl. Microbiol. Biotechnol. 58:138-146 (2002)). In a recent transcriptome analysis of Caulobacter crescentus, glutathione S-transferases and thioredoxin proteins were upregulated in response to cadmium and chromium stress (Hu, P. et al., J. Bacteriol. 187:8437-49 (2005)). In this study, two thioredoxin-coding transcripts (mpeB0233, mpeA1221) were upregulated (2.2, 2.0 respectively) when PM1 cells were grown on MTBE. Thioredoxin is a general protein disulfide reductase believed to serve as a cellular antioxidant by reducing the protein disulfide bonds elicited by oxidants such as heavy metals (Hu, P. et al., J. Bacteriol. 187:8437-49 (2005)).

Expression of several translation elongation factors (EF-G, mpeA3446; EF-P, mpeA1962; EF-Ts, mpeA1978; EF-Tu, mpeA1918, mpeA3458) was lower in MTBE-grown cells relative to ethanol-grown cells, suggesting lower translation efficiency for the former case. Furthermore, MTBE exposure significantly decreased expression of mpeA3491, predicted to encode the DnaK suppressor protein. This protein is similar to an RNA polymerase binding factor (DksA) shown to affect the efficiency of rRNA operon transcription depending on environmental conditions (Paul, B. J. et al., Proc. Natl. Acad. Sci. USA 102:7823-7828 (2005)). A dksA mutant of Shigella flexneri was shown to be more sensitive to oxidative damage (Mogull, S. A. et al., Infect. Immun. 69:5742-51 (2001)) and expression of a dksA homolog in R. sphaeroides was significantly downregulated in H₂O₂-treated cells compared to untreated cells (Zeller, T. et al., J. Bacteriol. 187:7232-7242 (2005)).

Several genes coding for components of the DNA repair system were significantly upregulated in response to MTBE exposure, including DNA repair exonuclease (mpeA2182, mpeA1721), DNA polymerase involved in SOS response (mpeB0048, mpeB0052, mpeA1533), alkylated DNA repair protein (mpeA3751), histone (mpeA3165), and restriction-modification system type I methyltransferase (mpeB0329) (Table 3). These genes may be involved in the ability of M. petroleiphilum PM1 to monitor its environment for changes and stresses, and to adjust its cellular physiology accordingly. It has been shown recently that at the level of single bacterial cells, expression of biodegradation genes requires transcriptional machinery that may also be poised to respond to abiotic stress (Cases, I. et al., Nat. Rev. Microbiol. 3:105-18 (2005)) (Velazquez, F. et al., Environ. Microbiol. 8:591-602 (2006)). In the case of P. putida mt-2, an m-xylene degrading bacterium, the presence m-xylene is sensed by the bacterium both as a C source and as an environmental stressor (Ramos, J. L. et al., Curr. Opin. Microbiol. 4:166-71 (2001)).

Methylotrophic metabolism. Compelling evidence for the linkage between MTBE and methanol oxidation pathways was the upregulation of genes involved in oxidation of formate. The genes coding for two of the three different types of formate dehydrogenases present in PM1 (Kane, S. R. et al., J. Bacteriol. 189:1931-1945 (2007)) were 1.5- to 6.3-fold upregulated in MTBE-grown cells compared to ethanol-grown cells (Table 1). Additionally, a gene mpeA3393 with unknown function, coding for a homolog of methanol dehydrogenase (XoxF/MxaF) was 2.9-fold upregulated. We observed also that expression of the tetrahydromethanopterin-dependent oxidative pathway (for conversion of the toxic product formaldehyde to CO₅) was greater in ethanol-grown cells exposed for 4 hr to MTBE relative to ethanol-grown control cells (data not shown), while MTBE-grown cells showed no differential expression of any of these genes. All the genes of the succinate dehydrogenase cluster were downregulated in MTBE relative to ethanol-grown cells (Table 1).

Transport. Genes presumably involved in Co and Ni (cbiOQMK) and Mn (mpeA0955) transport were upregulated in MTBE-grown cells compared to ethanol-grown cells (Table 1). The genome of PM1 also has a putative chemiosmotic antiporter efflux system similar to CzcCBA RND family transporters of Ralstonia metallidurans, conferring resistance to Cd, Zn and Co (Kane, S. R. et al., J. Bacteriol. 189:1931-1945 (2007)). Two of the genes cszA and cszB were upregulated in MTBE-grown cells in comparison to ethanol-grown cells. In physiology studies with PM1, the addition of elemental cobalt and iron to growth media substantially enhanced growth and degradation rates of MTBE and TBA (K. Hristova, unpublished data), suggesting that Fe and Co are cofactors of the MTBE/TBA oxidation system.

Genes possibly coding for resistance and reduction of arsenic were also upregulated following exposure to MTBE. Products of mpeA1583 and mpeA1357 are similar to arsenic reductase that is responsible for arsenate reduction to arsenite after which arsenite is translocated out of the cell by arsenite permease (mpeA1627). The results indicate that MTBE may trigger the expression of metal resistance genes (see Table S5 in the supplemental material). Similar trends, albeit in reversed situations, have been reported in the literature for other organisms and other organic contaminants; for example As(V) causes a clear stimulation of the transcription of the xy1 genes, responsible for m-xylene degradation by P. putida mt-2 (Velazquez, F. et al., Environ. Microbiol. 8:591-602 (2006)).

The PM1 genome encodes about 39 putative proteins involved in iron transport and homeostasis, which implies an importance of iron in its physiology. Only two genes mpeB0525, an Fe³⁺-dicitrate transporter and mpeA0955, an Fe²⁺/Mn²⁺ transporter were upregulated in MTBE-grown cells. In the PM1 genome we identified fep genes, which function in the synthesis of polypeptides required for uptake of ferric enterobactin. Surprisingly, we observed a significant downregulation of the genes coding for cobalamin/Fe³⁺ siderophore (cob/fep genes) and Fe uptake transporters (located on the PM1 megaplasmid and some of the corresponding genes located on the chromosome) in MTBE-grown compared to ethanol-grown cells. TonB/ExbB/D/TolQ transmembrane protein biopolymer transporters were also downregulated (significantly or slightly) in MTBE-grown cells. Since iron was not limiting under either growth condition, a possible explanation is that iron uptake could be downregulated under oxidative stress conditions in PM1 as a protective mechanism against further damage by ROS.

Several genes involved in the phosphonate transport and metabolisms were significantly upregulated in MTBE-grown compared to ethanol-grown cells (Table 1). Some multi-drug resistance efflux pump genes (mpeA1876), branched-chain amino acid transporters (mpeB0564-65, mpeA2036, mpeA1771, mpeA3675) and TRAP-type mannitol/chloroaromatic compound transport system genes (mpeA3655-56, mpeA2834) were also upregulated on MTBE. In contrast, some genes involved in nitrate transport (nark) were slightly upregulated, the majority of annotated sulfate transporters were downregulated, and the remaining genes involved in nitrate and sulfate transport were not differentially expressed on MTBE. Finally several genes coding for components of H+/Na+ ATPases were downregulated on MTBE (mpeA0190-98; see Table S5 in the supplemental material).

Regulators. When exposed to MTBE in the environment, PM1 has to reconcile competing signals for presence of a usable carbon source and presence of a toxic compound. Of the 307 identified putative regulatory genes, 24 were significantly upregulated and 34 were significantly downregulated in response to MTBE (see Table S5 in the supplemental material). An additional 18 regulatory genes were not included in the microarray data list, since they were not annotated in the early genome draft.

The chromosomally-encoded regulatory genes upregulated in MTBE-grown cells included those whose predicted products belong to AsnC, CRP, FIS, LysR, MerR, and TetR families, methyl-accepting chemotaxis proteins (MCP), serine/threonine protein kinases, as well as proteins containing GGDEF, EAL, PAS and PAC domains. The only upregulated regulatory genes located on the megaplasmid are the duplicates, mpeB0420/0456 and mpeB0434/0469, present on the 29 kb tandem repeat. While mpeB0420/0456 are predicted to encode a phosphonate regulator similar to PhnF of E. coli (GenBank accession number P16684), the predicted protein product of mpeB0434/0469 is a regulator of unknown function that contains the helix-turn-helix domain of the AraC family. However, the significance of the apparent upregulation of these genes is uncertain as only a single copy of each duplicate gene on the 29 kb tandem repeat was accounted for in the microarray.

The downregulated genes included 5 LuxR family regulators, 5 two-component response regulators, three sigma factors (mpeA0148, mpeA2106, mpeA2491) and 5 serine/threonine kinases/phosphatases (see Table S2 in the supplemental material). None of the downregulated regulators are located on the megaplasmid. The higher number of downregulated regulators may reflect the overall reduction in metabolic activity by PM1 in the presence of MTBE.

Motility genes. Motile bacteria are capable of chemotaxis, that is, they swim toward or away from specific environmental stimuli such as nutrients, toxic chemicals and oxygen concentration (Blair, D. F. Annu. Rev. Microbiol. 49:489-522 (1995)). Of the 125 identified motility genes in M. petroleiphilum, 20 showed significant upregulation, 22 showed significant downregulation, while 7 were not included in the microarray data set.

Overall the microarray data show that M. petroleiphilum did not undergo an obvious change in its capacity for swimming behavior, but did show a change in its potential chemotactic response when grown on MTBE. Four of the 14 methyl-accepting chemotaxis protein (MCP) genes were upregulated (mpeA0586, mpeA2780, mpeA3300, mpeA0935) and one was downregulated (mpeA2920) in MTBE-grown compared to ethanol-grown cells (Table S5 in the supplemental material). The mpeA0935 gene is immediately upstream and on the same strand as a predicted alcohol dehydrogenase mpeA0936. However, mpeA0936 did not show a significant change in expression, suggesting that its regulation is uncoupled from that of the MCP. Interestingly, of the two MCPs showing similarity to the aerotaxis protein Aer (GenBank accession number P50466), involved in sensing intracellular energy levels (Bibikov, S. I. et al., J. Bacteriol. 179:4075-4079 (1997); Rebbapragada, A. et al., Proc. Natl. Acad. Sci. USA 94:10541-10546 (1997)), one was upregulated (mpeA2780) and the other was strongly downregulated (mpeA2920).

### CONCLUDING REMARKS

In this study, high-density, whole-genome cDNA microarrays were used to investigate differential gene expression when M. petroleiphilum PM1 was grown on MTBE and ethanol as sole carbon sources. This is the first time that evidence has been presented linking all the steps of the MTBE degradation pathway with candidate genes. The microarray studies conducted thus far have led to interesting and testable hypotheses concerning plasmid- and chromosome-encoded genes that may function in each step of the MTBE degradation pathway and have led to interesting hypotheses regarding the acquisition and evolution of MTBE genes as well as the involvement of IS elements in these complex processes. To further elucidate the function of the PM1 TBA hydroxylase enzyme system, we are currently performing whole genome microarray studies with MTBE- and TBA-grown cells, using the completed genome annotation. In addition, gene knockout experiments are being focused on mdpA, mdpJ and mdpK to test the hypotheses developed from microarray, comparative genomic and proteomic analyses.

Overall expression results confirm the upregulation of more genes in total, as well as higher expression levels for energy metabolism and housekeeping genes in the presence of the higher energy yielding and less recalcitrant substrate-ethanol. In spite of this clear trend, the higher number of unknown genes expressed in the presence of MTBE points to a wealth of untapped information related to bacterial survival in the presence of a recalcitrant, toxic carbon source.

M. petroleiphilum PM1 is known to be a member of subsurface microbial communities at several gasoline contaminated sites. Given that many contaminated sites have mixtures of organic contaminants including BTEX compounds and fuel oxygenates, it is ideal that bioremediation technologies would utilize microorganisms capable of metabolizing the target contaminant, as well as other contaminants present. In PM1, the exposure to MTBE induces pathways for degradation of a spectrum of aromatic compounds, such as benzene, toluene, xylene (BTX), phenolic compounds, alkanes and alicyclic, aliphatic or aryl ketones. This result suggests that PM1 could co-express pathways for biodegradation of BTX and fuel oxygenates in the bioremediation of gasoline contaminated aquifers. In addition, the upregulation of unrelated biodegradation pathways and oxidative stress response genes suggest the presence of a biodegradation global regulatory network, the elucidation of which is imperative for better understanding of bacterial bioremediation of complex mixtures of contaminants.

The microarray data reported here suggests that achieving a balance in expression of metabolic pathways while minimizing damage associated with environmental stressors is one of the factor in the ecological success of M. petroleiphilum PM1 in subsurface environments. These results expand our understanding of the metabolic capabilities of M. petroleiphilum PM1 under conditions of MTBE pressure in subsurface gasoline-contaminated environments.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

```
INFORMAL SEQUENCE LISTING
SEQ ID NO: 1
MpeB0606 MRNA
atgatttcgagcttgccaaacaacgacagcggcgggtccggggagacgcgatatcgcgatatgaaacggtacgcatggcc
catcggcgttttgtggccgatgttgcccgccatcggcattgcggctgctcagttgaccgggaaggcagcgttctattggc
tcgcccccttcctgacttttgtcgtgattcccctcctcgacatggttatcggaagcagccaaaagaacccgccggaaagc
gcgatcaaagccctcgaggacgacaactactatcggaacctgacgttcgtcacggtgcccgttcactacctttcgatgat
cactggcgcctgggcggttggaacgctcgaccttaccgggctcaactatgcaggaatctgcatctccgtgggtcttgcca
atggtcttgcgatcgttacttcccacgagcttggtcataagaaggacgcactcgaacgctggatgtccaagatctgcttg
gcggtcacggcctacggccaatacatgatcgatcacaaccgtggtcatcatcgggatgttgctactcctgaggactccag
ttccgctcgcatgggtgagggcatctatttcttcgcgctgcgcgaactcccgtacacgggatttattcgaccctggcgcc
tggagaaggaacgcttggcgcgcagcggcaaaggtccctggacgctggagaacgaattcctccagccggctctgatctcg
ctggtcttctacggtgccctgatcgtctggctgggttcgagcatcatcccgtacctgcttgctaccgccttcggtggcta
ctggtttttggtgatcgcggactatatagaacactacgggttgcttcgtcagaagcttcctgacggacgttacgagcggg
tccgacctgagcactcatggaacactgatcacattgcctcgaatgtgatctatttccatgtgcagcgacactcggatcac
cacgcgtttccgacgcgtagctaccaagcccttcgtagctatagcgacgtacccacaatgccgtccggatatccggggat
gatttggctctgtcacatcccgccgctgtttcgagccgtcatggatccgcttttgctcaagcagtacgacggcgacctca
ccaagatcaacatcgatccagggaagcgcagcaagctattccgacgttatgccaatcagctggcgcagcccgtccgtgac
tga
SEQ ID NO: 2
MpeB0606 PROTEIN
Protein Length = 400
MISSLPNNDSGGSGETRYRDMKRYAWPIGVLWPMLPAIGIAAAQLTGKAAFYWLAPFLTFVVIPLLDMVIGSSQKNPPES
AIKALEDDNYYRNLTFVTVPVHYLSMITGAWAVGTLDLTGLNYAGICISVGLANGLAIVTSHELGHKKDALERWMSKICL
AVTAYGQYMIDENRGHHRDVATPEDSSSARMGEGIYFFALRELPYTGFIRPWRLEKERLARSGKGPWTLENEFLQPALIS
LVFYGALIVWLGSSIIPYLLATAFGGYWFLVIADYIEHYGLLRQKLPDGRYERVRPEHSWNTDHIASNVIYFHVQRHSDH
HAFPTRSYQALRSYSDVPTMPSGYPGMIWLCHIPPLFRAVMDPLLLKQYDGDLTKINIDPGKRSKLFRRYANQLAQPVRD
SEQ ID NO: 3
MpeB0602 MRNA
atgatcaaaaatttcaagaaatggcaatgcgttacgtgctccttcacctatgacgaagagctaggcatgccatctgacgg
aattcctcccggcacggcctgggaggacgttccggacgactggacttgtcccgactgctcatcgcccaaatccgattttc
agatggtggaaatctaa
SEQ ID NO: 4
MpeB0602 PROTEIN
Protein Length = 58
MIKNFKKWQCVTCSFTYDEELGMPSDGIPPGTAWEDVPDDWTCPDCSSPKSDFQMVEI
SEQ ID NO: 5
MpeB0597 MRNA
gtgatttctacagctcaagagcgcggagatacgagggcaatcctcgtcctcggagccggccaagctggtttccaggtcgc
tgcctcgttgcgggacttcggatacagagggtgtgtcacgctcgttggtgatgaaccacactggccctatcgccgtccgc
ccttgtcaaaggggtatctagaaggttccgactccgccggcacgctcgcgctgcggttgggggctaaccaggaaagcctg
gaactggtgatgcggcttgggaagaaggggctcgccatcgacaggtcgtcgaacatcgtcacgctggactcaggtgagag
aatagggtacgaccatctagtgattgctatgggcgccactccgcgggcacttcgtgttcccggggtgcatctcgaaggcg
tgctcagtttgcgcacagtcgagcatgcagaggcacttcgcaatctgttccgagaaccaggggacatggtggtgatcggt
ggcggcttcatcgggatggaagtggctgcagtggctgccaaagctggtcagcgtgtcacggtcgtggaggctgaggaccg
ggtcatgtcccgcgttgtcgccccggagatctctggctatgttgccagcgagcacgcagcgcatggtgtttcgatcatga
cgggtcgttgcgccgtggcctttcatggccgatcgggacgcgtctccgccgtagaacttgatgatggtgtacggttgccg
gctcgcattgttttggtgggagtcggggtatcgccgaacatcgctcttgcggaagaggccgctttgacggtcgataacgg
aattgtagtcgatggatctcttctcacttcagatgagcggataagtgctattggtgattgttctagtttccccagtgttc
atgcgcgtcggcgagtgcgacttgagtctgtgcagaacgcagttgaccaggccaagtacgtcgccggtcgactgactgga
atgatgggtgaagtctaccaaggtacaccgtgtttctggacacgccagtacagcacgtcgattcagattgccggaatcgg
cgacggtaacgacgagcgttgggtgagcggagaccccgcgtcgggcaaattctccatctttcgattcaatggtggcacgt
tgtcatgcgtagaatcggttaattcttcagcagaccatgcggctgttcggaagctgttcagtggtggcatgccgctacca
acgccccgggagttgaccgatgcgcagtttctgccaaagctctcgctcgagagagtagccgcagcggagtcctcctag
SEQ ID NO: 6
MpeB0597 PROTEIN
Protein Length = 425
VISTAQERGDTRAILVLGAGQAGFQVAASLRDFGYRGCVTLVGDEPNWPYRRPPLSKGYLEGSDSAGTLALRLGANQESL
ELVMRLGKKGLAIDRSSNIVTLDSGERIGYDHLVIAMGATPRALRVPGVHLEGVLSLRTVEHAEALRNLFREPGDMVVIG
GGFIGMEVAAVAAKAGQRVTVVEAEDRVMSRVVAPEISGYVASEHAAHGVSIMTGRCAVAFHGRSGRVSAVELDDGVRLP
ARIVLVGVGVSPNIALAEEAALTVDNGIVVDGSLLTSDERISAIGDCSSFPSVHARRRVRLESVQNAVDQAKYVAGRLTG
MMGEVYQGTPCFWTRQYSTSIQIAGIGDGNDERWVSGDPASGKFSIFRFNGGTLSCVESVNSSADHAAVRKLFSGGMPLP
TPRELTDAQFLPKLSLERVAAAESS
SEQ ID NO: 7
MpeB0601 MRNA
gtggagacgagaatgcataaagcggcctcctggattcttaagccggagcgctggaagttgcctgcggcgtttcgcactgt
gtctcggcctgtgtttgccgtgagcgccccggctggctatggcaagtcgaccctgctgagcgagtggagagaagaagtca
tcgcactgggttaccgtgtcgtttggctactggttgacggcgatgatcaggatggcgataagctcgcgatcgatttgctc
cacgccttttctccagccgatacggaacgatcgcagtcattggtcaacggcgttggtgaccgcgggaaacgcgccgtaat
catggccctggttgcggaaatggcttcacgccaggagcggactgtattgttcgtcgatgacgttcattggctgtcagaca
acacggccgcgacccttttacgcccgttaattcgtcaccagcctgaacgtatggcgttggtgttgagtgggcgtgccaat
ttgagttcgctttccagcgaggcggctctcgatcgccgcttacacgtcgttgaccatattcagttggcattcaaccagtc
ggatattgcacagattctgaggcagtacagtgtcaagccgagacaggcattggtacaggccatctatgaacgcagcgagg
gttggccagcgatcgtgcgactcattgcgatgactctgcacagcgatgaagaaagtcaaaataatctgttgcaaggcctg
ctggagcgaccacaggccatttcggagtacctgagcgaagttctcctatcgcagttgccggatcgcgctgctcagttgtt
gctctgcctcgcgatgttgcggcggttcaatggccgcttggtggctgctgcgacagaaatgagcgacgcagaggctgtcc
ttgccgaattgcagcggcgcgcgctccctattagtcgcagcaatgacgcaatgcttccgtatgcactgcatccaattgtg
cgagatttcctgttgataagaatacgccgacagggtatcaatcaaatcggcccatatgtcgagcgtgcactcgcctggtt
gactgacaatggtcgaatcgacgccgccatcgatctaagcttagacgtcggcaacgtcaagaacgctgcggcgttgatag
accactatgcgcgtacgatggcgcggtatcagggccgtcatgctacgttcctttattgggcgaacaagcttccactggag
gccctggcgcaattcccggagatcagggtgaagcaagcctggtcactcgcagtccttagacgagctgcagaggcgaaagc
cgtacttgctaaactcataattgaattcgccgaaccgacaaacagacccgatgcagcttcgcatgggttcgacgaccagc
gcttgagccgcataaggcaggcagttgaactggaaaggtgcaccgtacctaccctctgcgatcgagcacaggatgccgcg
ccatacgcacgaagctggttgtcgcgctggccggacgcagaacctatcgatctggctatcgctaacatcgtggtgggatg
cggtgcgatggccgacactgactttgaagtcagcctagcgcatctgcgaactgcccaacgatatgtagacgaatgcaagg
ggtactacgtcaaagcctgggtcgatatgtggctagcaacggttttaagcaagcaagggcgatatcgacaggccctttac
gagtgcgacgaggcagtgacagcggtcgccacgcatcttggtggggaaactgctgtggagatcatgctacacgcgatccg
agctccgctgctttacgagatgaatcggctggaagaagcgggtgcggcgcttgagcatggcctcactgcactcattgagc
aaacctcggtcgactccattattatgggccacgttgcgctagcgaggctgcagaacgctcaaggctcccatctcgacgca
ctcgaaacgcttgctgaaggtgaagtgatcggcaggacgcacggtctgtcccggctagtcgtggctttggcggcagagcg
gattgacctccttctgagacatcgcgagcttggccaagcgcaggcacagtggctagagttgcagaacttctcggagtgcg
gtcccgccgatgcgtttgagagcgcgatgtctgacaaggccccacgcatcgaaagccggatagcgttgttgaaggggaac
aattcagttgcctgcgaactcaccgagcctgcgttacagcgagcgattcggaccggccagaaaaggaagcaggtcgaact
tctattgattcgggcccttgccgcccaggccgggaaggaacatgagagggctggggacgctcttcaaagggctatcgaag
tcgcgatgtcggaaggttatgtccgagtattcgtagacgagggtgagcagatgcggttgctgcttatctccgctgcggga
ttggcggcccgggccagttctcctaccggcgagtatctgcgccagatcctggctgccttcagtgttcaaaagagcgatcc
aaagacgtcggcgttcatagccggggccgagtcgcttacggggcgtgagctgaaaatcttgcggaggttgcaatcggacc
tatccaatcggcagcttgccgatacgctgtacatcaccgagggcacactgaaatggcacttgaagaacatttacggaaag
ttgaacgtgactaaccgcctgacggctgtcaccgcagggagaaaacttgggctgttagatagttga
SEQ ID NO: 8
MpeB0601 PROTEIN
Protein Length = 901
VETRMHKAASWILKPERWKLPAAFRTVSRPVFAVSAPAGYGKSTLLSEWREEVIALGYRVVWLLVDGDDQDGDKLAIDLL
HAFSPADTERSQSLVNGVGDRGKRAVIMALVAEMASRQERTVLFVDDVHWLSDNTAATLLRPLIRHQPERMALVLSGRAN
LSSLSSEAALDRRLHVVDHIQLAFNQSDIAQILRQYSVKPRQALVQAIYERSEGWPAIVRLIAMTLHSDEESQNNLLQGL
LERPQAISEYLSEVLLSQLPDRAAQLLLCLAMLRRFNGRLVAAATEMSDAEAVLAELQRRALPISRSNDAMLPYALHPIV
RDFLLIRIRRQGINQIGPYVERALAWLTDNGRIDAAIDLSLDVGNVKNAAALIDHYARTMARYQGRHATFLYWANKLPLE
ALAQFPEIRVKQAWSLAVLRRAAEAKAVLAKLIIEFAEPTNRPDAASHGFDDQRLSRIRQAVELERCTVPTLCDRAQDAA
PYARSWLSRWPDAEPIDLAIANIVVGCGAMADTDFEVSLAHLRTAQRYVDECKGYYVKAWVDMWLATVLSKQGRYRQALY
ECDEAVTAVATHLGGETAVEIMLHAIRAPLLYEMNRLEEAGAALEHGLTALIEQTSVDSIIMGHVALARLQNAQGSNLDA
LETLAEGEVIGRTHGLSRLVVALAAERIDLLLRHRELGQAQAQWLELQNFSECGPADAFESAMSDKAPRIESRIALLKGN
NSVACELTEPALQRAIRTGQRRKQVELLLIRALAAQAGKEHERAGDALQRAIEVAMSEGYVRVFVDEGEQMRLLLISAAG
LAARASSPTGEYLRQILAAFSVQKSDPKTSAFIAGAESLTGRELKILRRLQSDLSNRQLADTLYITEGTLKWHLKNIYGK
LNVTNRLTAVTAGRKLGLLDS
SEQ ID NO: 9
MpeB0558 MRNA
atgaacgcaccgatcattaagaaggttcttgtcgacagcggcgagctgaggatgaaggtggccggattgttccaggcggt
cggagtgtcgcccgagcatgcggaccagatcgccgaggtcgtcgtcttcgccgatctgcgcggcgtcgagtcgcacgggg
tccagttcacgccgcgatacgtccgcggcatcgcccgcggccacctgaacccgaagccggacatccgcgtcgtccaccga
cgcggcgcggtcgccgtcgtcgatgccgacaacggactgggcttcctgtcggcgcgccgcgcgatgaaggaggccatggc
gatcgccgccgaacacggcagcggctcggtggcggtgcgcaacagcaaccacttcggaccggcggccttctacccgatga
tggcgctggaggcgggaatgatcggctacgccacgcccgacggccccccccacacggtcgtgtggggcagccgcaggccg
gtgctttcgaacgacccagtcggctgggccttccccaccctcgaaggcctgccgatcgtcgtggacaccgcgtttaccgg
cgtgaaggagaagatcagattggccgcccagcgcggcggcacgatcccggccgattgggccgtcgggcccgacggcaatc
cgacgaccgacccgaaggtcgcgctcgagggttaccttctgcccatcggccagcacaagggctcggcgctgatcatcgcc
aacgaggtcgtctgcggcgccttggccggcgccctcttcagcttcgaagtgtcgccgaagctcgtgatgggtgcggacca
tcacgcttcatggaagtgcggccacttcgtccaggcgttggatccgggcgccttcggcgaccgcgacgcattcttgcgcc
gcaccagcgagttggcgtcggctctgcgcaacgcgccgcgcgccgagggcgtgcagcgcatctacatgcccggcgagatc
gaggccgaactgtcggcgcagcgcttgcgggacgggctgccgctggcggtcaccacgctcgaggccctcgacgccgtggc
ccgcgaggtcggcgcgcccgtaccgtcggcgccgctggccacgcgcgagatgccgtga
SEQ ID NO: 10
MpeB0558 PROTEIN
Protein Length = 365
MNAPIIKKVLVDSGELRMKVAGLFQAVGVSPEHADQIAEVVVFADLRGVESHGVQFTPRYVRGIARGHLNPKPDIRVVHR
RGAVAVVDADNGLGFLSARRAMKEAMAIAAEHGSGSVAVRNSNHFGPAAFYPMMALEAGMIGYATTDGPPHTVVWGSRRP
VLSNDPVGWAFPTLEGLPIVVDTAFTGVKEKIRLAAQRGGTIPADWAVGPDGNPTTDPKVALEGYLLPIGQHKGSALIIA
NEVVCGALAGALFSFEVSPKLVMGADHHDSWKCGHFVQALDPGAFGDRDAFLRRTSELASALRNAPRAEGVQRIYMPGEI
EAELSAQRLRDGLPLAVTTLEALDAVAREVGAPVPSATLATREMP
SEQ ID NO: 11
MpeA2443 MRNA
atgccagttgatgcacacgcacaaggactgctggatgccctcaaggcgcagggcctcaagtcgttcgaacagatgaccat
cgccgaggcgcgcggcgcgatcgagacgttcgtgggcctgcaggctccgccagaggaggtgaagcaagtccacgatctga
cggtgaaggggcctgcaggtgagctccagtaccggatcttcgttcccgctggtccgacacctatgccggttctcgtgtac
ttccacgggggcggctgggtcggtgggagtctcgcggtggtggacgaaccctgccgggcgatcgcgaaccgttgcggcgc
cgtggtcatcgctgcgagctaccgactttcaccggaagcccggttccccgcggcgacggacgacgcgtacgccgcagtcc
aatgggccagcgccaacgccgcgacctacggcggtgatgcgagccgtctgggcgtcatgggcgacagcgccggcgccaat
atcgcggcggttgtttcaatgatggcgcgtgatcgcaaggggccggccatcaaggctcagatcctgacctatcccgtgat
ccagcgcgatggcgacttcgcctcccgcaaagccaatgaagaggggtatctgctgacgtcggcgggtgtcgcgtggttct
ggaagcagtacctggcgagcgatgcggacgcggtcaacccgtacgcatcgcccatcatggccaaggacctgaccggcctg
ccccctgcactggtgatgaccgccgaattcgaccccgcgcgcgacgaaggcgaggcctacggcaaggcgctggccaaggc
gggggttcctgtgacggtccgcaggttcgaaggtctgatccacggcgtcttcggaagcgctgatcaacccgcttcgtga
SEQ ID NO: 12
MpeA2443 PROTEIN
Protein Length = 292
MPVDAHAQGLLDALKAQGLKSFEQMTIAEARGAIETFVGLQAPPEEVKQVHDLTVKGPAGELQYRIFVPAGPTPMPVLVY
FHGGGWVGGSLAVVDEPCRAIANRCGAVVIAASYRLSPEARFPAATDDAYAAVQWASANAATYGGDASRLGVMGDSAGAN
LAAVVSMMARDRKGPAIKAQILTYPVIQRDGDFASRKANEEGYLLTSAGVAWFWKQYLASDADAVNPYASPIMAKDLTGL
PPALVNTAEFDPARDEGEAYGKALAKAGVPVTVRRFEGLIHGVFGSADQPAS
SEQ ID NO: 13
MpeB0555 MRNA
atgggtaacagagagcctttggccgcggccgggcagggcacagcctacagcgggtaccggctgcgcgacctgcagaatgc
cgcccccacgaacctggaaatccttcgtacgggccccggcacgccgatgggcgagtacatgcgccgctactggcagcccg
tatgcctgtcgcaggaactgaccgacgtgcccaaggcgatccggatcctgcacgaggatctggtggcattcagggaccgc
cagggcaacgtcggcgtgctgcaccgcaagtgcgcccaccgcggggcctcgctcgagttcggcatcgtgcaggaacgcgg
gatccgctgctgctaccacggttggcacttcgacgtcgacggcaaactgctggaggcgccggcggaaccccccgacacca
agctgaaggaaaccgtctgccagggcgcctatccggccttcgagcgcgacggcctggtgttcgcctacatggggccggcg
gatcgcagaccggagttcccggtgttcgacggctacgtgttgccgaagggaacgcggttgattccgttctccaatgtctt
cgactgcaactggcttcaggtctacgaaaaccagatcgaccactaccacaccgcgctgctgcacaacaacatgacggtcg
ccggcgtggactcgaagctggccgacggcgcgacgctgcaggggggcttcggcgagatgccaatcatcgactggcacccg
accgacgacaacaacggcatgatcttcaccgccggccggcgcctgtcggacgacgaagtctggatccgaatctcgcagat
gggcctgccgaactggatgcagaacgccgccatcgtggcggcggcgccgcagcgacactccggcccggcgatgtcgcgtt
ggcaggtgccggtcgacgacgagcactcgatcgccttcggctggcgccacttcaacgacgaggtggacccggagcaccgt
ggaagggaagaggagtgcggggtcgacaagatcgactttctgatcggtcagacccggcatcggccttatgaagagaggca
gcgggttccgggcgactacgaagccatcgtcagccaggggccgatagccgtccacggccttgagcatcccggccggtcgg
acgtgggtgtgtacatgtgtcgctcgctgcttcgcgacgctgtggccggcaaggcgccgcccgacccggtgcgcgtgaag
gctgggtcgaccgatgggcaaacgctgccgcgatacgcgtcggacagtcgactgcggatccgccgccggccgagccggga
agcggacagtgacgtcatccgcaaggccgcgcaccaggttttcgcgatcatgaaggagtgcgacgaactgccggtcgtgc
agcgcaggccgcatgtcctgcggcgcctcgacgagatcgaagcgagcctctga
SEQ ID NO: 14
MpeB0555 PROTEIN
Protein Length = 470
MGNREPLAAAGQGTAYSGYRLRDLQNAAPTNLEILRTGPGTPMGEYMRRYWQPVCLSQELTDVPKAIRILHEDLVAFRDR
QGNVGVLHRKCAHRGASLEFGIVQERGIRCCYHGWHFDVDGKLLEAPAEPPDTKLKETVCQGAYPAFERDGLVFAYMGPA
DRRPEFPVFDGYVLPKGTRLIPFSNVFDCNWLQVYENQIDHYHTALLHNNMTVAGVDSKLADGATLQGGFGEMPIIDWHP
TDDNNGMIFTAGRRLSDDEVWIRISQMGLPNWMQNAAIVAAAPQRHSGPAMSRWQVPVDDEHSIAFGWRHFNDEVDPEHR
GREEECGVDKIDFLIGQTRHRPYEERQRVPGDYEAIVSQGPIAVHGLEHPGRSDVGVYMCRSLLRDAVAGKAPPDPVRVK
AGSTDGQTLPRYASDSRLRIRRRPSREADSDVIRKAAHQVFAIMKECDELPVVQRRPHVLRRLDEIEASL
SEQ ID NO: 15
MpeB0554 MRNA
atgtatcagttgagtcacaccggcaagtacccgaagacggcgctgaacctgcgggtccggcagatcacctaccaggggat
cggcatcaacgcctacgaattcgtgcgcgaggacggcggcgaactggaggagttcaccgccggggcccacgtggatctgt
acttccgcgacggacgcgtgcgacagtattcgttgtgcaacgaccccgccgagcgtcggcgatacctgatcgcggtgctg
cgcgacgacaatgggcgcgggggttccatcgcgatccacgaacgcgtgcacacgcaacgactcgtcgcggtcggacaccc
gcgcaacaacttcccgctgattgagggggcgccccaccagatcctgctggccggcggcatcggcatcacgccgctgaagg
ccatggtgcatcggttggaaaggataggcgcggactacaccctgcactactgcgcgaagtcgagcgcccacgcggcgttc
caggaggaactcgcgccgctggccgccaaggggcgcgtgatcatgcacttcgacggcggcaatccggccaagggcctcga
catcgcggcgctgctgcggcggtacgagccgggttggcagctctactactgcggcccccccgggttcatggaggcctgca
cacgtgcctgcaccaattggcccgccgaggcggtgcacttcgagtacttcgtcggcgcgccggtgcttcccgccgaggga
gtcccccacgacatcggcagcgatgcgctggcgctcgggttccagatcaagatcgccagcacgggaacggtcctgacggt
accgaacgacaagtcgatcgcgcaggtgcttggcgagcacggcatcgaagtaccgacatcatgccagagcggcctgtgcg
gtacgtgcaaggtccgctatctcgcgggcgacgtcgagcatcgggattacttgctgtccgccgaggcacgcacgcagttc
ctgaccacctgcgtgtcgcgctcgaagggcgcgacgctggtcctggatctttga
SEQ ID NO: 16
MpeB0554 PROTEIN
Protein Length = 337
MYQLSHTGKYPKTALNLRVRQITYQGIGINAYEFVREDGGELEEFTAGAHVDLYFRDGRVRQYSLCNDPAERRRYLIAVL
RDDNGRGGSIAIHERVHTQRLVAVGHPRNNFPLIEGAPHQILLAGGIGITPLKAMVHRLERIGADYTLHYCAKSSAHAAF
QEELAPLAAKGRVIMHFDGGNPAKGLDIAALLRRYEPGWQLYYCGPPGFMEACTRACTNWPAEAVHFEYFVGAPVLPAEG
VPHDIGSDALALGFQIKIASTGTVLTVPNDKSIAQVLGEHGIEVPTSCQSGLCGTCKVRYLAGDVEHRDYLLSAEARTQF
LTTCVSRSKGATLVLDL
SEQ ID NO: 17
MpeB0561 MRNA
atgaaagagatcggcctaatcggccttggaaacatcggcggcggaatgtgccggcgccttctcgaccgcggcatcggcgt
cgtcgggttcgacctttcgccggcggccacgaaagccgccgcggaacacggcgcccggatcgaggtcagccccgcggcgg
tcgcgcagcaggttgatgtcgttgtcacgtcgctgccgaatccccccatcgtgcgtgacgtctacctgggcaaacagggt
ctggtcgcgcaggcgcggccagggagcacgctgatcgagaccagcaccatcgacccgaacaccattcgtgaggtcgcgca
ggcggcgaccaagtccggcatccggatcctcgacatcgcactgtccggcgagccgccgcaggcggtccttggcgaactgg
tcttccaggtgggtggccccgacgagttgatcgaccagcatctcgagttgctgcaggtgctggcgaagaagatcaaccgc
acgggcggcattgggaccgccaagacggtcaagctcgtgaacaacctgatgtcgctgggcaacgtcgctgtggccgccga
ggctttcgtcctgggcgtgaagtgcgggatggaaccgaagcggttgtacgagatcctgtccgtctcgggcggacgctcgg
cgcacttcatcagcgggttccagaaggtcatcgaaggcgactacggcgccagcttcaagaccagcctggcgctgaaggac
atcaacctcattctcgacctcgccaacgaggagcactacgcggcgcggctcgcgccggtcatcgcatcgctgtaccgcga
cgccgttgggcgagggctgggggaagagaacttcacgtcggtggtcaagggctacgaagccactgcaggcattcgcgttg
ccgagtccggctag
SEQ ID NO: 18
MpeB0561 PROTEIN
Protein Length = 297
MKEIGLIGLGNIGGGMCRRLLDRGIGVVGFDLSPAATKAAAEHGARIEVSPAAVAQQVDVVVTSLPNPPIVRDVYLGKQG
LVAQARPGSTLIETSTIDPNTIREVAQAATKSGIRILDIALSGEPPQAVLGELVFQVGGPDELIDQHLELLQVLAKKINR
TGGIGTAKTVKLVNNLMSLGNVAVAAEAFVLGVKCGMEPKRLYEILSVSGGRSAHFISGFQKVIEGDYGASFKTSLALKD
INLILDLANEEHYAARLAPVIASLYRDAVGRGLGEENFTSVVKGYEATAGIRVAESG
SEQ ID NO: 19
MpeA0361 MRNA
atggtcaccgaatacagaaactacatcgacggcgagttcctggccaaccgctcgggcgccctgatcgacgtgcacaaccc
ggccacccacgagctgctcgcccgtgtgcccgacgccccgaacgacgtcgtcgacctggccgtgcaggccgcacgcaccg
cgcagccggggtgggcgaagctgcccgcgatccagcgcgcccagcacctgcgtgccatcgccgcccggctgcgcgagaac
gtggaggaactggcccacaccatcaccgccgagcagggcaaggtgctgggtctggcgcgcgtggaggtgaacttcaccgc
cgactacatggactacatggccgagtgggcgcgccgcctcgagggcgaggtgctcaccagtgaccgcgtcggcgagagca
tcttcctgatgcgcaagccgatcggcgtggccgccggcatcctgccgtggaacttcccgttcttcctgatcgcgcgcaag
ctggcgccggcgctgatcaccggcaacaccatcgtgatcaagccgagcgagatcacgccgatcaacgccttcgagttcgc
gcgcctggcctcgcagaccgacctgccgcgcggcgtgttcaacctggtgggcggcaccggcgccggcgccggcgcgcagc
tcacctcgcaccgcgacgtgggcatcgtgtcgttcaccggcagcgtggagaccggcacgcgcatcatgaccgcggcgtcg
aagaacctcacgcgcgtgaacctcgagctcggcggcaaggcaccggccatcgtgctggccgacgccgacctcgacctggc
ggtgaaggccatctacgactcgcgcgtgatcaacaccggacaggtgtgcaactgcgccgagcgcgtgtacgtgcagcgca
aggtggccgacgagttcaccagcaagatcgccgcgcgcatggccggcacgctgtacggcgacccgctggcccagcccgac
gtggcgatgggtccgctggtcagccaggccggcctcgacaaggtggcgggcatggtggaccgcgcccgcgcggccggcgc
cagcatcgtgcaaggtggccgcaaggccaaccgcgacaagggctaccactacgagcccaccgtcatcgcgaactgcagcg
ccgacatggagatcatgcgcaaggagatcttcgggccggtgctgccgatccaggtggtggacgagctcgacgaggcgatc
gcgctggcgaacgactccgactacggcctgacctcgtcgatcttcaccaaggacctgaactcggccatgcgcgcggtgcg
cgacctgcagttcggcgagacctacgtgaaccgcgagcacttcgaggcgatgcagggcttccacgccggccgcaagaagt
cgggcatcggcggggccgatggcaagcacggcctgtacgagttcaccgagacgcacgtggtctacatccagcacggctga
SEQ ID NO: 20
MpeA0361 PROTEIN
Protein Length = 479
MVTEYRNYIDGEFLANRSGALIDVHNPATHELLARVPDAPNDVVDLAVQAARTAQPGWAKLPAIQRAQHLPAIAARLREN
VEELAHTITAEQGKVLGLARVEVNFTADYMDYMAEWARRLEGEVLTSDRVGESIFLMRKPIGVAAGILPWNFPFFLIARK
LAPALITGNTIVIKPSEITPINAFEFARLASQTDLPRGVFNLVGGTGAGAGAQLTSHRDVGIVSFTGSVETGTRIMTAAS
KNLTRVNLELGGKAPAIVLADADLDLAVKAIYDSRVINTGQVCNCAERVYVQRKVADEFTSKIAARMAGTLYGDPLAQPD
VAMGPLVSQAGLDKVAGMVDRARAAGASIVQGGRKANRDKGYHYEPTVIANCSADMEIMRKEIFGPVLPIQVVDELDEAI
ALANDSDYGLTSSIFTKDLNSAMRAVRDLQFGETYVNREHFEAMQGFHAGRKRSGIGGADGKHGLYEFTETHVVYIQHG
SEQ ID NO: 21
MpeB0541 MRNA
atgacctggcttgagccgcagataaagtcccaactccaatcggagcgcaaggactgggaagcgaacgaagtcggcgcctt
cttgaagaaggcgcccgagcgcaaggagcagttccacacgatcggggacttcccggtccagcgcacctacaccgctgccg
acatcgccgacacgccgctggaggacatcggtcttccggggcgctacccgttcacgcgcgggccctacccgacgatgtac
cgcagccgcacctggacgatgcgccagatcgccggcttcggcaccggcgaggacaccaacaagcgcttcaagtatctgat
cgcgcagggccagaccggcatctccaccgacttcgacatgcccacgctgatgggctacgactccgaccacccgatgagcg
acggcgaggtcggccgcgagggcgtggcgatcgacacgctggccgacatggaggcgctgctggccgacatcgacctcgag
aagatctcggtctcgttcacgatcaacccgagcgcctggatcctgctcgcgatgtacgtggcgctcggcgagaagcgcgg
ctacgacctgaacaagctgtcgggcacggtgcaggccgacatcctgaaggagtacatggcgcagaaggagtacatctacc
cgatcgcgccgtcggtgcgcatcgtgcgcgacatcatcacctacagcgcgaagaacctgacgcgctacaacccgatcaac
atctcgggctaccacatcagcgaggccggctcgtcgccgctgcaggaggcggccttcacgctggccaacctgatcaccta
cgtgaacgaggtgacggagaccggcatgcacgtcgacgagttcgcgccgcgcctcgccttcttcttcgtgtcgcaaggtg
acttcttcgaggaggtagcgaagttccgcgccctacgtcgctgctacgcgaagatcatgaaggagcgcttcggcgcgaag
aaccccgagtcgatgcggctgcgctttcactgtcagaccgcggcggcgactttgaccaagccgcagtacatggtcaacgt
cgtgcgtacgtcgctgcaggcgctgtcggccgtgctcggcggcgcgcagtcgctgcacaccaacggctacgacgaagcct
tcgcgatcccgaccgaggatgcgatgaagatggcgctgcgcacgcagcagatcattgccgaggagagtggtgtcgccgac
gtgatcgacccgctgggtggcagctactacgtcgaggcgctgaccaccgagtacgagaagaagatcttcgagatcctcga
ggaagtcgagaagcgcggtggcaccatcaagctgatcgagcagggctggttccagaagcagattgcggacttcgcttacg
agaccgcgctgcgcaagcagtccggccagaagccggtgatcggggtgaaccgcttcgtcgagaacgaagaggacgtcaag
atcgagatccacccgtacgacaacacgacggccgaacgccagatttcccgcacgcgccgcgttcgcgccgagcgcgacga
ggccaaggtgcaagcgatgctcgaccaactggtggctgtcgccaaggacgagtcccagaacctgatgccgctgaccatcg
aactggtgaaggccggcgcaacgatgggggacatcgtcgagaagctgaaggggatctggggtacctaccgcgagacgccg
gtcttctga
SEQ ID NO: 22
MpeB0541 PROTEIN
Protein Length = 562
MTWLEPQIKSQLQSERKDWEANEVGAFLKKAPERKEQFHTIGDFPVQRTYTAADIADTPLEDIGLPGRYPFTRGPYPTMY
RSRTWTMRQIAGFGTGEDTNKRFKYLIAQGQTGISTDFDMPTLMGYDSDHPMSDGEVGREGVAIDTLADMEALLADIDLE
KISVSFTINPSAWILLANYVALGEKRGYDLNKLSGTVQADILKEYMAQKEYIYPIAPSVRIVRDIITYSAKNLKRYNPIN
ISGYHISEAGSSPLQEAAFTLANLITYVNEVTETGMHVDEFAPRLAFFFVSQGDFFEEVAKFRALRRCYAKIMKERFGAK
NPESMRLRFHCQTAAATLTKPQYMVNVVRTSLQALSAVLGGAQSLHTNGYDEAFAIPTEDAMKMALRTQQIIAEESGVAD
VIDPLGGSYYVEALTTEYEKKIFEILEEVEKRGGTIKLIEQGWFQKQIADFAYETALRKQSGQKPVIGVNRFVENEEDVK
IEIHPYDNTTAERQISRTRRVRAERDEAKVQAMLDQLVAVAKDESQNLMPLTIELVKAGATMGDIVEKLKGIWGTYRETP
VF
SEQ ID NO: 23
MpeB0538 MRNA
atggaccaaatcccgatccgcgttcttctcgccaaagtcggcctcgacggccatgacagaggcgtcaaggtggtcgctcg
cgcgctgcgcgacgccggcatggacgtcatctactccggccttcatcgcacgcccgaagaagtggtcaacaccgccatcc
aggaagacgtggacgtgctgggtgtgagcctcctgtccggcgtgcagctcacggtcttccccaagatcttcaagctcctg
gaagagagaggcgccggcgacttgatcgtgatcgccggtggcgtgatgccggacgaggacgccgcggccatccgcaaact
gggcgtgcgcgaggtgctcctgcaggacacgccgccgcaggccatcatcgactcgatccgcgccttggtcgccgcgcgcg
gcgcccgctga
SEQ ID NO: 24
Mpe50538 PROTEIN
Protein Length = 136
MDQIPIRVLLAKVGLDGHDRGVKVVARALRDAGMDVIYSGLHRTPEEVVNTAIQEDVDVLGVSLLSGVQLTVFPKIFKLL
EERGAGDLIVIAGGVMPDEDAAAIRKLGVREVLLQDTPPQAIIDSIRALVAARGAR
SEQ ID NO: 25
MpeB0547 MRNA
atggcaaaccctccaggctccatcggcgtcatcggcgccggcaccatgggcaacggaatcgcgcaggtctgcgcggtggc
cggcctcaacgtgacgatgttggacgtcgacgacgccgcgttgaagcgcggcatggacaccatcatccgcaatctcgacc
gcatggtggcgaaagagaagctgacggccagcgcccgcgatgccgcgctggcgaagatcagtaccggtctggactatggc
gcgctgcagtccgccgatatggtgatcgaggctgcgacggagaacctgggactcaagctgaagatcctgcggcaagtcgc
caactgcgtcggcaaggacgcgatcattgcgacgaacacctcgtcgatctcgatcacccagctgggcgctgtgctcgacg
cgccggagtgcttcattggcatccactttttcaatcccgtgccgctgatgtcgctgctggaggtcatccgcggcgtgcag
acgtcggacgcgacccatgctgccacgatggcgtttgcccagaaggtgggcaaggcgccgatcacggtccgcaacagccc
cggtttcgtggtcaatcgcatcctgtgcccgatgatcaacgaggccatcttcgtcctgcaggaaggcctggcgtctgccg
aaggcattgatgtcggcatgcgcctgggatgcaaccatccgatcggtccgctagcactggccgacatgatcggcctcgac
accttgttgtcgatcatgggcgtgctttacgacgagtttaacgatcccaagtaccgcccagcgctgctgctgaaggagat
ggtcgccgccggccgcctcggccggaagaccaagcaagggttctacagctactcctga
SEQ ID NO: 26
MpeB0547 PROTEIN
Protein Length = 285
MANPPGSIGVIGAGTMGNGIAQVCAVAGLNVTMLDVDDAALKRGMDTIIRNLDRMVAKEKLTASARDAALAKISTGLDYG
ALQSADMVIEAATENLGLKLKILRQVANCVGKDAIIATNTSSISITQLGAVLDAPECFIGIHFFNPVPLMSLLEVIRGVQ
TSDATHAATMAFAQKVGKAPITVRNSPGFVVNRILCPMINEAIFVLQEGLASAEGIDVGMRLGCNHPIGPLALADMIGLD
TLLSIMGVLYDEFNDPKYRPALLLKEMVAAGRLGRKTKQGFYSYS
SEQ ID NO: 27
MpeA3367 MRNA
atgtccaccgatgatccggtcgtgatcgtgtcggctgcgcgaacgccgatcggcgggttgctcggcgacctggcggcgct
ggcggcctgggaactgggcgccgtcgcgatccgcgccgcggtcgaacgcgccggcgtgccgggcgacgccgtcgacgagg
tgctgatgggcaattgcctgatggcgggccagggccaggcgccggcccgccaggcggcgcgcaaggccggccttccggac
tcggccggcgcggtgacgctgtcgaagatgtgcggctccggcatgcgcgcgctgatgttcggccatgacatgctggcggc
cggctcggccgaggtggtggtggccggcggcatggagagcatgacgaacgcaccgcacctgagcttcgtgcgcaaggggc
tgaagtacggcgcggcggtgctgtacgaccacatggcgctcgacggcctggaggacgcctacgagcgcggcaagtcgatg
ggcgtattcgccgaacagtgcgtcagctattacagcttccggcgcgaggcgatggacgcgttcgcggtggcgtcgacgca
gcgcgcgatcgcggcccacaacgacggcagcttcgactgggagatcgcgccggtcacgctggccggcagggcgggcgacg
tgaccgtcgaccgcgacgagcagcccttcaaggccaagctcgacaagatcacggcgctgaagccggccttcggcaaggac
ggcacgatcaccgccgccacctcgtcgagcatctccgacggcgccgcggcgctggtgctgatgcgtgcctccaccgcccg
cgcgcgcggcctcgcgccgatcgccgtgctgcgcgcgcacgcggtgcatgcgcaggcgccggcctggttctccaccgcgc
cggccggcgcgatccgcaaggtgctgcagaagaccggctggtcggtgcgcgacgtcgacctgtgggagatcaacgaggcc
ttcgccgcggtgacgatggcggcgatgaccgatttcgagctgccgcacgagcgtgtcaacgtgcacggcggggcctgcgc
gctgggccacccgatcggcgcgtcgggggcccgcatcgtcgtgacgctgctgggcgcgctgcagcggcgcgggctgcggc
gtggcgtggcggcgctgtgcatcggcggcggcgaggccacggcactggcggtcgagctgccttga
SEQ ID NO: 28
MpeA3367 PROTEIN
Protein Length = 394
MSTDDPVVIVSAARTPIGGLLGDLAALAAWELGAVAIRAAVERAGVPGDAVDEVLMGNCLMAGQGQAPARQAARKAGLPD
SAGAVTLSKMCGSGMRALMFGHDMLAAGSAEVVVAGGMESMTNAPHLSFVRKGLKYGAAVLYDHMALDGLEDAYERGKSM
GVFAEQCVSYYSFRREAMDAFAVASTQRAIAAHNDGSFDWEIAPVTLAGRAGDVTVDRDEQPFKAKLDKITALKPAFGKD
GTITAATSSSISDGAAALVLMRASTARARGLAPIAVLRAHAVHAQAPAWFSTAPAGAIRKVLQKTGWSVRDVDLWEINEA
FAAVTMAAMTDFELPHERVNVHGGACALGHPIGASGARIVVTLLGALQRRGLRRGVAALCIGGGEATALAVELP
SEQ ID NO: 29
MpeBO539 protein
    1 meewkfpvey denylppads rywfprretm paaerdkail grlqqvcqya wdtspfyrrk
  61 weeanfhpsq lksledfetr vpvikktdlr esqaahppfg dyvcvpdsei fhvhgtsgtt
121 grptafgigr adwraianah arimwgmgir pgdlvcvaav fslymgswga lagaerlrak
181 afpfgagapg msarlvqwld tmkpaafygt psyaihlaev areeklnprd fglkclffsg
241 epgasvpgvk drieeaygak vydcgsmaem spfmnvagte qsndgmlcwq diiytevcdp
301 anmrrvpygq rgtpvythle rtsqpmirll sgdltlwtnd enpcgrtypr lpqgifgrid
361 dmftirgeni ypseidaaln qmsgyggehr ivitresamd elllrvepse svhaagaaal
421 etfraeashr vqtvlgvrak velvapnsia rtdfkarrvi ddrdvfraln qqlqssa
```

What is claimed is:

1. A method for modulating Methyl tertiary-butyl ether (MTBE) degradation, the method comprising modulating expression of a polypeptide selected from the group consisting of alkane 1-mono-oxygenase, dehydrogenase, tert-butyl alcohol hydroxylase, 2-methyl-2-hydroxy-1-propanol dehydrogenase, hydroxyisobutyraldehyde dehydrogenase, 2-hydroxy-isobutyryl-CoA ligase, 2-hydroxy-isobutyryl-CoA mutase, 3-hydroxy-butryl-CoA dehydrogenase, and combinations thereof.

2. The method of claim 1, wherein the MTBE-mono-oxygenase, dehydrogenase is encoded by a nucleic acid comprising the sequence set forth in SEQ ID NO: 1; the tert-butyl alcohol hydroxylase is encoded by a nucleic acid comprising the sequence set forth in SEQ ID NO: 13 or 15; the 2-methyl-2-hydroxy-1-propanol dehydrogenase is encoded by a nucleic acid comprising the sequence set forth in SEQ ID NO: 17, hydroxyisobutyraldehyde dehydrogenase is encoded by a nucleic acid comprising the sequence set forth in SEQ ID NO: 19, the 2-hydroxy-isobutyryl-CoA mutase is encoded by a nucleic acid comprising the sequence set forth in SEQ ID NO:21 or 23, and the 3-hydroxy-butryl-CoA dehydrogenase is encoded by a nucleic acid comprising the sequence set forth in SEQ ID NO:25.

3. The method of claim 1, wherein the MTBE-mono-oxygenase, dehydrogenase is encoded by a nucleic acid encoding the sequence set forth in SEQ ID NO: 2; the tert-butyl alcohol hydroxylase is encoded by a nucleic acid encoding the sequence set forth in SEQ ID NO: 14 or 16; the 2-methyl-2-hydroxy-1-propanol dehydrogenase is encoded by a nucleic acid encoding the sequence set forth in SEQ ID NO: 18, hydroxyisobutyraldehyde dehydrogenase is encoded by a nucleic acid encoding the sequence set forth in SEQ ID NO: 20, the 2-hydroxy-isobutyryl-CoA ligase is encoded by a nucleic acid encoding the sequence set forth in SEQ ID NO:29, the 2-hydroxy-isobutyryl-CoA mutase is encoded by a nucleic acid encoding the sequence set forth in SEQ ID NO:22 or 24, and the 3-hydroxy-butryl-CoA dehydrogenase is encoded by a nucleic acid encoding the sequence set forth in SEQ ID NO:26.

4. A method for identifying a compound that modulates MTBE degradation, the method comprising
(i) contacting a compound with a nucleic acid encoding a polypeptide selected from the group consisting of MTBE-mono-oxygenase, dehydrogenase, tert-butyl alcohol hydroxylase, 2-methyl-2-hydroxy-1-propanol dehydrogenase, hydroxyisobutyraldehyde dehydrogenase, 2-hydroxy-isobutyryl-CoA ligase, 2-hydroxy-isobutyryl-CoA mutase, 3-hydroxy-butryl-CoA dehydrogenase; and
(ii) determining the effect of the compound upon the polypeptide, wherein a compound that increases or decreases the expression of the nucleic acid is identified as a compound that modulates MTBE degradation.

5. The method of claim 4, wherein the effect is determined in vitro.

6. The method of claim 4, wherein the nucleic acid is expressed in a host cell.

7. The method of claim 6, wherein the host cell is E. coli.

8. The method of claim 4, wherein the polypeptide is recombinant.

9. The method of claim 4, wherein the compound is a small organic molecule

10. An isolated polynucleotide comprising the sequence set forth in SEQ ID NOS:1, 3, 5, 7, 9, 11, 13, 15, 17, 19, 21, 23, 25, or 27

11. An expression vector comprising a polynucleotide of claim 10 operably linked to an expression control sequence.

12. A host cell comprising an expression vector according to claim 11.

13. The host cell of claim 12, wherein the cell is E. coli.

14. An isolated polypeptide comprising an amino acid sequence encoded by a polynucleotide of claim 10.

15. An isolated polypeptide comprising the sequence set forth in SEQ ID NO: 2, 4, 6, 8, 10, 12, 14, 16, 18, 20, 22, 24, 26, 28 or 29.


SEQUENCE LISTING (36 sequences)

SEQ ID NO: 1 - DNA, 1203 bp, Methylibium petroleiphilum
  Methylibium petroleiphilum strain PM1 plasmid RPME01 methyl tert-butyl ether (MTBE) monooxygenase, alkane-1-monooxygenase, MpeB0606, MTBE degradation pathway mdpA
atgatttcgagcttgccaaacaacgacagcggcgggtccggggagacgcgatatcgcgatatgaaacggtacgcatggcccatcggcgttttgtggccgatgttgcccgccatcggcattgcggctgctcagttgaccgggaaggcagcgttctattggctcgcccccttcctgacttttgtcgtgattcccctcctcgacatggttatcggaagcagccaaaagaacccgccggaaagcgcgatcaaagccctcgaggacgacaactactatcggaacctgacgttcgtcacggtgcccgttcactacctttcgatgatcactggcgcctgggcggttggaacgctcgaccttaccgggctcaactatgcaggaatctgcatctccgtgggtcttgccaatggtcttgcgatcgttacttcccacgagcttggtcataagaaggacgcactcgaacgctggatgtccaagatctgcttggcggtcacggcctacggccaatacatgatcgatcacaaccgtggtcatcatcgggatgttgctactcctgaggactccagttccgctcgcatgggtgagggcatctatttcttcgcgctgcgcgaactcccgtacacgggatttattcgaccctggcgcctggagaaggaacgcttggcgcgcagcggcaaaggtccctggacgctggagaacgaattcctccagccggctctgatctcgctggtcttctacggtgccctgatcgtctggctgggttcgagcatcatcccgtacctgcttgctaccgccttcggtggctactggtttttggtgatcgcggactatatagaacactacgggttgcttcgtcagaagcttcctgacggacgttacgagcgggtccgacctgagcactcatggaacactgatcacattgcctcgaatgtgatctatttccatgtgcagcgacactcggatcaccacgcgtttccgacgcgtagctaccaagcccttcgtagctatagcgacgtacccacaatgccgtccggatatccggggatgatttggctctgtcacatcccgccgctgtttcgagccgtcatggatccgcttttgctcaagcagtacgacggcgacctcaccaagatcaacatcgatccagggaagcgcagcaagctattccgacgttatgccaatcagctggcgcagcccgtccgtgactga

SEQ ID NO: 2 - protein, 400 aa, Methylibium petroleiphilum
  Methylibium petroleiphilum strain PM1 plasmid RPME01 methyl tert-butyl ether (MTBE) monooxygenase, alkane-1-monooxygenase, MpeB0606, MTBE degradation pathway MdpA
MISSLPNNDSGGSGETRYRDMKRYAWPIGVLWPMLPAIGIAAAQLTGKAAFYWLAPFLTFVVIPLLDMVIGSSQKNPPESAIKALEDDNYYRNLTFVTVPVHYLSMITGAWAVGTLDLTGLNYAGICISVGLANGLAIVTSHELGHKKDALERWMSKICLAVTAYGQYMIDHNRGHHRDVATPEDSSSARMGEGIYFFALRELPYTGFIRPWRLEKERLARSGKGPWTLENEFLQPALISLVFYGALIVWLGSSIIPYLLATAFGGYWFLVIADYIEHYGLLRQKLPDGRYERVRPEHSWNTDHIASNVIYFHVQRHSDHHAFPTRSYQALRSYSDVPTMPSGYPGMIWLCHIPPLFRAVMDPLLLKQYDGDLTKINIDPGKRSKLFRRYANQLAQPVRD

SEQ ID NO: 3 - DNA, 177 bp, Methylibium petroleiphilum
  Methylibium petroleiphilum strain PM1 plasmid RPME01 rubredoxin, MpeB0602, MTBE degradation pathway mdpB
atgatcaaaaatttcaagaaatggcaatgcgttacgtgctccttcacctatgacgaagagctaggcatgccatctgacggaattcctcccggcacggcctgggaggacgttccggacgactggacttgtcccgactgctcatcgcccaaatccgattttcagatggtggaaatctaa

SEQ ID NO: 4 - protein, 58 aa, Methylibium petroleiphilum
  Methylibium petroleiphilum strain PM1 plasmid RPME01 rubredoxin, MpeB0602, MTBE degradation pathway MdpB
MIKNFKKWQCVTCSFTYDEELGMPSDGIPPGTAWEDVPDDWTCPDCSSPKSDFQMVEI

SEQ ID NO: 5 - DNA, 1278 bp, Methylibium petroleiphilum
  Methylibium petroleiphilum strain PM1 plasmid RPME01 rubredoxin reductase, putative ferredoxin reductase, MpeB0597, MTBE degradation pathway mdpD
gtgatttctacagctcaagagcgcggagatacgagggcaatcctcgtcctcggagccggccaagctggtttccaggtcgctgcctcgttgcgggacttcggatacagagggtgtgtcacgctcgttggtgatgaaccacactggccctatcgccgtccgcccttgtcaaaggggtatctagaaggttccgactccgccggcacgctcgcgctgcggttgggggctaaccaggaaagcctggaactggtgatgcggcttgggaagaaggggctcgccatcgacaggtcgtcgaacatcgtcacgctggactcaggtgagagaatagggtacgaccatctagtgattgctatgggcgccactccgcgggcacttcgtgttcccggggtgcatctcgaaggcgtgctcagtttgcgcacagtcgagcatgcagaggcacttcgcaatctgttccgagaaccaggggacatggtggtgatcggtggcggcttcatcgggatggaagtggctgcagtggctgccaaagctggtcagcgtgtcacggtcgtggaggctgaggaccgggtcatgtcccgcgttgtcgccccggagatctctggctatgttgccagcgagcacgcagcgcatggtgtttcgatcatgacgggtcgttgcgccgtggcctttcatggccgatcgggacgcgtctccgccgtagaacttgatgatggtgtacggttgccggctcgcattgttttggtgggagtcggggtatcgccgaacatcgctcttgcggaagaggccgctttgacggtcgataacggaattgtagtcgatggatctcttctcacttcagatgagcggataagtgctattggtgattgttctagtttccccagtgttcatgcgcgtcggcgagtgcgacttgagtctgtgcagaacgcagttgaccaggccaagtacgtcgccggtcgactgactggaatgatgggtgaagtctaccaaggtacaccgtgtttctggacacgccagtacagcacgtcgattcagattgccggaatcggcgacggtaacgacgagcgttgggtgagcggagaccccgcgtcgggcaaattctccatctttcgattcaatggtggcacgttgtcatgcgtagaatcggttaattcttcagcagaccatgcggctgttcggaagctgttcagtggtggcatgccgctaccaacgccccgggagttgaccgatgcgcagtttctgccaaagctctcgctcgagagagtagccgcagcggagtcctcctag

SEQ ID NO: 6 - protein, 425 aa, Methylibium petroleiphilum
  Methylibium petroleiphilum strain PM1 plasmid RPME01 rubredoxin reductase, putative ferredoxin reductase, MpeB0597, MTBE degradation pathway MdpD
VISTAQERGDTRAILVLGAGQAGFQVAASLRDFGYRGCVTLVGDEPHWPYRRPPLSKGYLEGSDSAGTLALRLGANQESLELVMRLGKKGLAIDRSSNIVTLDSGERIGYDHLVIAMGATPRALRVPGVHLEGVLSLRTVEHAEALRNLFREPGDMVVIGGGFIGMEVAAVAAKAGQRVTVVEAEDRVMSRVVAPEISGYVASEHAAHGVSIMTGRCAVAFHGRSGRVSAVELDDGVRLPARIVLVGVGVSPNIALAEEAALTVDNGIVVDGSLLTSDERISAIGDCSSFPSVHARRRVRLESVQNAVDQAKYVAGRLTGMMGEVYQGTPCFWTRQYSTSIQIAGIGDGNDERWVSGDPASGKFSIFRFNGGTLSCVESVNSSADHAAVRKLFSGGMPLPTPRELTDAQFLPKLSLERVAAAESS

SEQ ID NO: 7 - DNA, 2706 bp, Methylibium petroleiphilum
  Methylibium petroleiphilum strain PM1 plasmid RPME01 ATP-dependent transcriptional regulator-like protein protein, MpeB0601, MTBE degradation pathway mdpC
gtggagacgagaatgcataaagcggcctcctggattcttaagccggagcgctggaagttgcctgcggcgtttcgcactgtgtctcggcctgtgtttgccgtgagcgccccggctggctatggcaagtcgaccctgctgagcgagtggagagaagaagtcatcgcactgggttaccgtgtcgtttggctactggttgacggcgatgatcaggatggcgataagctcgcgatcgatttgctccacgccttttctccagccgatacggaacgatcgcagtcattggtcaacggcgttggtgaccgcgggaaacgcgccgtaatcatggccctggttgcggaaatggcttcacgccaggagcggactgtattgttcgtcgatgacgttcattggctgtcagacaacacggccgcgacccttttacgcccgttaattcgtcaccagcctgaacgtatggcgttggtgttgagtgggcgtgccaatttgagttcgctttccagcgaggcggctctcgatcgccgcttacacgtcgttgaccatattcagttggcattcaaccagtcggatattgcacagattctgaggcagtacagtgtcaagccgagacaggcattggtacaggccatctatgaacgcagcgagggttggccagcgatcgtgcgactcattgcgatgactctgcacagcgatgaagaaagtcaaaataatctgttgcaaggcctgctggagcgaccacaggccatttcggagtacctgagcgaagttctcctatcgcagttgccggatcgcgctgctcagttgttgctctgcctcgcgatgttgcggcggttcaatggccgcttggtggctgctgcgacagaaatgagcgacgcagaggctgtccttgccgaattgcagcggcgcgcgctccctattagtcgcagcaatgacgcaatgcttccgtatgcactgcatccaattgtgcgagatttcctgttgataagaatacgccgacagggtatcaatcaaatcggcccatatgtcgagcgtgcactcgcctggttgactgacaatggtcgaatcgacgccgccatcgatctaagcttagacgtcggcaacgtcaagaacgctgcggcgttgatagaccactatgcgcgtacgatggcgcggtatcagggccgtcatgctacgttcctttattgggcgaacaagcttccactggaggccctggcgcaattcccggagatcagggtgaagcaagcctggtcactcgcagtccttagacgagctgcagaggcgaaagccgtacttgctaaactcataattgaattcgccgaaccgacaaacagacccgatgcagcttcgcatgggttcgacgaccagcgcttgagccgcataaggcaggcagttgaactggaaaggtgcaccgtacctaccctctgcgatcgagcacaggatgccgcgccatacgcacgaagctggttgtcgcgctggccggacgcagaacctatcgatctggctatcgctaacatcgtggtgggatgcggtgcgatggccgacactgactttgaagtcagcctagcgcatctgcgaactgcccaacgatatgtagacgaatgcaaggggtactacgtcaaagcctgggtcgatatgtggctagcaacggttttaagcaagcaagggcgatatcgacaggccctttacgagtgcgacgaggcagtgacagcggtcgccacgcatcttggtggggaaactgctgtggagatcatgctacacgcgatccgagctccgctgctttacgagatgaatcggctggaagaagcgggtgcggcgcttgagcatggcctcactgcactcattgagcaaacctcggtcgactccattattatgggccacgttgcgctagcgaggctgcagaacgctcaaggctcccatctcgacgcactcgaaacgcttgctgaaggtgaagtgatcggcaggacgcacggtctgtcccggctagtcgtggctttggcggcagagcggattgacctccttctgagacatcgcgagcttggccaagcgcaggcacagtggctagagttgcagaacttctcggagtgcggtcccgccgatgcgtttgagagcgcgatgtctgacaaggccccacgcatcgaaagccggatagcgttgttgaaggggaacaattcagttgcctgcgaactcaccgagcctgcgttacagcgagcgattcggaccggccagaaaaggaagcaggtcgaacttctattgattcgggcccttgccgcccaggccgggaaggaacatgagagggctggggacgctcttcaaagggctatcgaagtcgcgatgtcggaaggttatgtccgagtattcgtagacgagggtgagcagatgcggttgctgcttatctccgctgcgggattggcggcccgggccagttctcctaccggcgagtatctgcgccagatcctggctgccttcagtgttcaaaagagcgatccaaagacgtcggcgttcatagccggggccgagtcgcttacggggcgtgagctgaaaatcttgcggaggttgcaatcggacctatccaatcggcagcttgccgatacgctgtacatcaccgagggcacactgaaatggcacttgaagaacatttacggaaagttgaacgtgactaaccgcctgacggctgtcaccgcagggagaaaacttgggctgttagatagttga

SEQ ID NO: 8 - protein, 901 aa, Methylibium petroleiphilum
  Methylibium petroleiphilum strain PM1 plasmid RPME01 ATP-dependent transcriptional regulator-like protein protein, MpeB0601, MTBE degradation pathway MdpC
VETRMHKAASWILKPERWKLPAAFRTVSRPVFAVSAPAGYGKSTLLSEWREEVIALGYRVVWLLVDGDDQDGDKLAIDLLHAFSPADTERSQSLVNGVGDRGKRAVIMALVAEMASRQERTVLFVDDVHWLSDNTAATLLRPLIRHQPERMALVLSGRANLSSLSSEAALDRRLHVVDHIQLAFNQSDIAQILRQYSVKPRQALVQAIYERSEGWPAIVRLIAMTLHSDEESQNNLLQGLLERPQAISEYLSEVLLSQLPDRAAQLLLCLAMLRRFNGRLVAAATEMSDAEAVLAELQRRALPISRSNDAMLPYALHPIVRDFLLIRIRRQGINQIGPYVERALAWLTDNGRIDAAIDLSLDVGNVKNAAALIDHYARTMARYQGRHATFLYWANKLPLEALAQFPEIRVKQAWSLAVLRRAAEAKAVLAKLIIEFAEPTNRPDAASHGFDDQRLSRIRQAVELERCTVPTLCDRAQDAAPYARSWLSRWPDAEPIDLAIANIVVGCGAMADTDFEVSLAHLRTAQRYVDECKGYYVKAWVDMWLATVLSKQGRYRQALYECDEAVTAVATHLGGETAVEIMLHAIRAPLLYEMNRLEEAGAALEHGLTALIEQTSVDSIIMGHVALARLQNAQGSHLDALETLAEGEVIGRTHGLSRLVVALAAERIDLLLRHRELGQAQAQWLELQNFSECGPADAFESAMSDKAPRIESRIALLKGNNSVACELTEPALQRAIRTGQKRKQVELLLIRALAAQAGKEHERAGDALQRAIEVAMSEGYVRVFVDEGEQMRLLLISAAGLAARASSPTGEYLRQILAAFSVQKSDPKTSAFIAGAESLTGRELKILRRLQSDLSNRQLADTLYITEGTLKWHLKNIYGKLNVTNRLTAVTAGRKLGLLDS

SEQ ID NO: 9 - DNA, 1098 bp, Methylibium petroleiphilum
  Methylibium petroleiphilum strain PM1 plasmid RPME01 hydroxymethyl tert-butyl ether dehydrogenase, L-sulfolactate dehydrogenase, MpeB0558, MTBE degradation pathway mdpE
atgaacgcaccgatcattaagaaggttcttgtcgacagcggcgagctgaggatgaaggtggccggattgttccaggcggtcggagtgtcgcccgagcatgcggaccagatcgccgaggtcgtcgtcttcgccgatctgcgcggcgtcgagtcgcacggggtccagttcacgccgcgatacgtccgcggcatcgcccgcggccacctgaacccgaagccggacatccgcgtcgtccaccgacgcggcgcggtcgccgtcgtcgatgccgacaacggactgggcttcctgtcggcgcgccgcgcgatgaaggaggccatggcgatcgccgccgaacacggcagcggctcggtggcggtgcgcaacagcaaccacttcggaccggcggccttctacccgatgatggcgctggaggcgggaatgatcggctacgccacgaccgacggccccccccacacggtcgtgtggggcagccgcaggccggtgctttcgaacgacccagtcggctgggccttccccaccctcgaaggcctgccgatcgtcgtggacaccgcgtttaccggcgtgaaggagaagatcagattggccgcccagcgcggcggcacgatcccggccgattgggccgtcgggcccgacggcaatccgacgaccgacccgaaggtcgcgctcgagggttaccttctgcccatcggccagcacaagggctcggcgctgatcatcgccaacgaggtcgtctgcggcgccttggccggcgccctcttcagcttcgaagtgtcgccgaagctcgtgatgggtgcggaccatcacgattcatggaagtgcggccacttcgtccaggcgttggatccgggcgccttcggcgaccgcgacgcattcttgcgccgcaccagcgagttggcgtcggctctgcgcaacgcgccgcgcgccgagggcgtgcagcgcatctacatgcccggcgagatcgaggccgaactgtcggcgcagcgcttgcgggacgggctgccgctggcggtcaccacgctcgaggccctcgacgccgtggcccgcgaggtcggcgcgcccgtaccgtcggcgacgctggccacgcgcgagatgccgtga

SEQ ID NO: 10 - protein, 365 aa, Methylibium petroleiphilum
  Methylibium petroleiphilum strain PM1 plasmid RPME01 hydroxymethyl tert-butyl ether dehydrogenase, L-sulfolactate dehydrogenase, MpeB0558, MTBE degradation pathway MdpE
MNAPIIKKVLVDSGELRMKVAGLFQAVGVSPEHADQIAEVVVFADLRGVESHGVQFTPRYVRGIARGHLNPKPDIRVVHRRGAVAVVDADNGLGFLSARRAMKEAMAIAAEHGSGSVAVRNSNHFGPAAFYPMMALEAGMIGYATTDGPPHTVVWGSRRPVLSNDPVGWAFPTLEGLPIVVDTAFTGVKEKIRLAAQRGGTIPADWAVGPDGNPTTDPKVALEGYLLPIGQHKGSALIIANEVVCGALAGALFSFEVSPKLVMGADHHDSWKCGHFVQALDPGAFGDRDAFLRRTSELASALRNAPRAEGVQRIYMPGEIEAELSAQRLRDGLPLAVTTLEALDAVAREVGAPVPSATLATREMP

SEQ ID NO: 11 - DNA, 879 bp, Methylibium petroleiphilum
  Methylibium petroleiphilum strain PM1 plasmid RPME01 tert-butyl formate carboxylesterase, putative lipase, MpeA2443
atgccagttgatgcacacgcacaaggactgctggatgccctcaaggcgcagggcctcaagtcgttcgaacagatgaccatcgccgaggcgcgcggcgcgatcgagacgttcgtgggcctgcaggctccgccagaggaggtgaagcaagtccacgatctgacggtgaaggggcctgcaggtgagctccagtaccggatcttcgttcccgctggtccgacacctatgccggttctcgtgtacttccacgggggcggctgggtcggtgggagtctcgcggtggtggacgaaccctgccgggcgatcgcgaaccgttgcggcgccgtggtcatcgctgcgagctaccgactttcaccggaagcccggttccccgcggcgacggacgacgcgtacgccgcagtccaatgggccagcgccaacgccgcgacctacggcggtgatgcgagccgtctgggcgtcatgggcgacagcgccggcgccaatctcgcggcggttgtttcaatgatggcgcgtgatcgcaaggggccggccatcaaggctcagatcctgacctatcccgtgatccagcgcgatggcgacttcgcctcccgcaaagccaatgaagaggggtatctgctgacgtcggcgggtgtcgcgtggttctggaagcagtacctggcgagcgatgcggacgcggtcaacccgtacgcatcgcccatcatggccaaggacctgaccggcctgccccctgcactggtgatgaccgccgaattcgaccccgcgcgcgacgaaggcgaggcctacggcaaggcgctggccaaggcgggggttcctgtgacggtccgcaggttcgaaggtctgatccacggcgtcttcggaagcgctgatcaacccgcttcgtga

SEQ ID NO: 12 - protein, 292 aa, Methylibium petroleiphilum
  Methylibium petroleiphilum strain PM1 plasmid RPME01 tert-butyl formate carboxylesterase, putative lipase, MpeA2443
MPVDAHAQGLLDALKAQGLKSFEQMTIAEARGAIETFVGLQAPPEEVKQVHDLTVKGPAGELQYRIFVPAGPTPMPVLVYFHGGGWVGGSLAVVDEPCRAIANRCGAVVIAASYRLSPEARFPAATDDAYAAVQWASANAATYGGDASRLGVMGDSAGANLAAVVSMMARDRKGPAIKAQILTYPVIQRDGDFASRKANEEGYLLTSAGVAWFWKQYLASDADAVNPYASPIMAKDLTGLPPALVMTAEFDPARDEGEAYGKALAKAGVPVTVRRFEGLIHGVFGSADQPAS

SEQ ID NO: 13 - DNA, 1413 bp, Methylibium petroleiphilum
  Methylibium petroleiphilum strain PM1 plasmid RPME01 tert-butyl alcohol hydroxylase, phthalate 4,5-dioxygenase, MpeB0555, MTBE degradation pathway mdpJ
atgggtaacagagagcctttggccgcggccgggcagggcacagcctacagcgggtaccggctgcgcgacctgcagaatgccgcccccacgaacctggaaatccttcgtacgggccccggcacgccgatgggcgagtacatgcgccgctactggcagcccgtatgcctgtcgcaggaactgaccgacgtgcccaaggcgatccggatcctgcacgaggatctggtggcattcagggaccgccagggcaacgtcggcgtgctgcaccgcaagtgcgcccaccgcggggcctcgctcgagttcggcatcgtgcaggaacgcgggatccgctgctgctaccacggttggcacttcgacgtcgacggcaaactgctggaggcgccggcggaaccccccgacaccaagctgaaggaaaccgtctgccagggcgcctatccggccttcgagcgcgacggcctggtgttcgcctacatggggccggcggatcgcagaccggagttcccggtgttcgacggctacgtgttgccgaagggaacgcggttgattccgttctccaatgtcttcgactgcaactggcttcaggtctacgaaaaccagatcgaccactaccacaccgcgctgctgcacaacaacatgacggtcgccggcgtggactcgaagctggccgacggcgcgacgctgcaggggggcttcggcgagatgccaatcatcgactggcacccgaccgacgacaacaacggcatgatcttcaccgccggccggcgcctgtcggacgacgaagtctggatccgaatctcgcagatgggcctgccgaactggatgcagaacgccgccatcgtggcggcggcgccgcagcgacactccggcccggcgatgtcgcgttggcaggtgccggtcgacgacgagcactcgatcgccttcggctggcgccacttcaacgacgaggtggacccggagcaccgtggaagggaagaggagtgcggggtcgacaagatcgactttctgatcggtcagacccggcatcggccttatgaagagaggcagcgggttccgggcgactacgaagccatcgtcagccaggggccgatagccgtccacggccttgagcatcccggccggtcggacgtgggtgtgtacatgtgtcgctcgctgcttcgcgacgctgtggccggcaaggcgccgcccgacccggtgcgcgtgaaggctgggtcgaccgatgggcaaacgctgccgcgatacgcgtcggacagtcgactgcggatccgccgccggccgagccgggaagcggacagtgacgtcatccgcaaggccgcgcaccaggttttcgcgatcatgaaggagtgcgacgaactgccggtcgtgcagcgcaggccgcatgtcctgcggcgcctcgacgagatcgaagcgagcctctga

SEQ ID NO: 14 - protein, 470 aa, Methylibium petroleiphilum
  Methylibium petroleiphilum strain PM1 plasmid RPME01 tert-butyl alcohol hydroxylase, phthalate 4,5-dioxygenase, MpeB0555, MTBE degradation pathway MdpJ
MGNREPLAAAGQGTAYSGYRLRDLQNAAPTNLEILRTGPGTPMGEYMRRYWQPVCLSQELTDVPKAIRILHEDLVAFRDRQGNVGVLHRKCAHRGASLEFGIVQERGIRCCYHGWHFDVDGKLLEAPAEPPDTKLKETVCQGAYPAFERDGLVFAYMGPADRRPEFPVFDGYVLPKGTRLIPFSNVFDCNWLQVYENQIDHYHTALLHNNMTVAGVDSKLADGATLQGGFGEMPIIDWHPTDDNNGMIFTAGRRLSDDEVWIRISQMGLPNWMQNAAIVAAAPQRHSGPAMSRWQVPVDDEHSIAFGWRHFNDEVDPEHRGREEECGVDKIDFLIGQTRHRPYEERQRVPGDYEAIVSQGPIAVHGLEHPGRSDVGVYMCRSLLRDAVAGKAPPDPVRVKAGSTDGQTLPRYASDSRLRIRRRPSREADSDVIRKAAHQVFAIMKECDELPVVQRRPHVLRRLDEIEASL

SEQ ID NO: 15 - DNA, 1014 bp, Methylibium petroleiphilum
  Methylibium petroleiphilum strain PM1 plasmid RPME01 putative iron-sulfur oxidoreductase subunit, MpeB0554, MTBE degradation pathway mdpK
atgtatcagttgagtcacaccggcaagtacccgaagacggcgctgaacctgcgggtccggcagatcacctaccaggggatcggcatcaacgcctacgaattcgtgcgcgaggacggcggcgaactggaggagttcaccgccggggcccacgtggatctgtacttccgcgacggacgcgtgcgacagtattcgttgtgcaacgaccccgccgagcgtcggcgatacctgatcgcggtgctgcgcgacgacaatgggcgcgggggttccatcgcgatccacgaacgcgtgcacacgcaacgactcgtcgcggtcggacacccgcgcaacaacttcccgctgattgagggggcgccccaccagatcctgctggccggcggcatcggcatcacgccgctgaaggccatggtgcatcggttggaaaggataggcgcggactacaccctgcactactgcgcgaagtcgagcgcccacgcggcgttccaggaggaactcgcgccgctggccgccaaggggcgcgtgatcatgcacttcgacggcggcaatccggccaagggcctcgacatcgcggcgctgctgcggcggtacgagccgggttggcagctctactactgcggcccccccgggttcatggaggcctgcacacgtgcctgcaccaattggcccgccgaggcggtgcacttcgagtacttcgtcggcgcgccggtgcttcccgccgagggagtcccccacgacatcggcagcgatgcgctggcgctcgggttccagatcaagatcgccagcacgggaacggtcctgacggtaccgaacgacaagtcgatcgcgcaggtgcttggcgagcacggcatcgaagtaccgacatcatgccagagcggcctgtgcggtacgtgcaaggtccgctatctcgcgggcgacgtcgagcatcgggattacttgctgtccgccgaggcacgcacgcagttcctgaccacctgcgtgtcgcgctcgaagggcgcgacgctggtcctggatctttga

SEQ ID NO: 16 - protein, 337 aa, Methylibium petroleiphilum
  Methylibium petroleiphilum strain PM1 plasmid RPME01 putative iron-sulfur oxidoreductase subunit, MpeB0554, MTBE degradation pathway MdpK
MYQLSHTGKYPKTALNLRVRQITYQGIGINAYEFVREDGGELEEFTAGAHVDLYFRDGRVRQYSLCNDPAERRRYLIAVLRDDNGRGGSIAIHERVHTQRLVAVGHPRNNFPLIEGAPHQILLAGGIGITPLKAMVHRLERIGADYTLHYCAKSSAHAAFQEELAPLAAKGRVIMHFDGGNPAKGLDIAALLRRYEPGWQLYYCGPPGFMEACTRACTNWPAEAVHFEYFVGAPVLPAEGVPHDIGSDALALGFQIKIASTGTVLTVPNDKSIAQVLGEHGIEVPTSCQSGLCGTCKVRYLAGDVEHRDYLLSAEARTQFLTTCVSRSKGATLVLDL

SEQ ID NO: 17 - DNA, 894 bp, Methylibium petroleiphilum
  Methylibium petroleiphilum strain PM1 plasmid RPME01 2-methyl-2-hydroxy-1-propanol dehydrogenase, 3-hydroxyisobutyrate dehydrogenase, MpeB0561, MTBE degradation pathway mdpH
atgaaagagatcggcctaatcggccttggaaacatcggcggcggaatgtgccggcgccttctcgaccgcggcatcggcgtcgtcgggttcgacctttcgccggcggccacgaaagccgccgcggaacacggcgcccggatcgaggtcagccccgcggcggtcgcgcagcaggttgatgtcgttgtcacgtcgctgccgaatccccccatcgtgcgtgacgtctacctgggcaaacagggtctggtcgcgcaggcgcggccagggagcacgctgatcgagaccagcaccatcgacccgaacaccattcgtgaggtcgcgcaggcggcgaccaagtccggcatccggatcctcgacatcgcactgtccggcgagccgccgcaggcggtccttggcgaactggtcttccaggtgggtggccccgacgagttgatcgaccagcatctcgagttgctgcaggtgctggcgaagaagatcaaccgcacgggcggcattgggaccgccaagacggtcaagctcgtgaacaacctgatgtcgctgggcaacgtcgctgtggccgccgaggctttcgtcctgggcgtgaagtgcgggatggaaccgaagcggttgtacgagatcctgtccgtctcgggcggacgctcggcgcacttcatcagcgggttccagaaggtcatcgaaggcgactacggcgccagcttcaagaccagcctggcgctgaaggacatcaacctcattctcgacctcgccaacgaggagcactacgcggcgcggctcgcgccggtcatcgcatcgctgtaccgcgacgccgttgggcgagggctgggggaagagaacttcacgtcggtggtcaagggctacgaagccactgcaggcattcgcgttgccgagtccggctag

SEQ ID NO: 18 - protein, 297 aa, Methylibium petroleiphilum
  Methylibium petroleiphilum strain PM1 plasmid RPME01 2-methyl-2-hydroxy-1-propanol dehydrogenase, 3-hydroxyisobutyrate dehydrogenase, MpeB0561, MTBE degradation pathway MdpH
MKEIGLIGLGNIGGGMCRRLLDRGIGVVGFDLSPAATKAAAEHGARIEVSPAAVAQQVDVVVTSLPNPPIVRDVYLGKQGLVAQARPGSTLIETSTIDPNTIREVAQAATKSGIRILDIALSGEPPQAVLGELVFQVGGPDELIDQHLELLQVLAKKINRTGGIGTAKTVKLVNNLMSLGNVAVAAEAFVLGVKCGMEPKRLYEILSVSGGRSAHFISGFQKVIEGDYGASFKTSLALKDINLILDLANEEHYAARLAPVIASLYRDAVGRGLGEENFTSVVKGYEATAGIRVAESG

SEQ ID NO: 19 - DNA, 1440 bp, Methylibium petroleiphilum
  Methylibium petroleiphilum strain PM1 plasmid RPME01 hydroxyisobutyraldehyde dehydrogenase, lactaldehyde dehydrogenase, MpeA0361
atggtcaccgaatacagaaactacatcgacggcgagttcctggccaaccgctcgggcgccctgatcgacgtgcacaacccggccacccacgagctgctcgcccgtgtgcccgacgccccgaacgacgtcgtcgacctggccgtgcaggccgcacgcaccgcgcagccggggtgggcgaagctgcccgcgatccagcgcgcccagcacctgcgtgccatcgccgcccggctgcgcgagaacgtggaggaactggcccacaccatcaccgccgagcagggcaaggtgctgggtctggcgcgcgtggaggtgaacttcaccgccgactacatggactacatggccgagtgggcgcgccgcctcgagggcgaggtgctcaccagtgaccgcgtcggcgagagcatcttcctgatgcgcaagccgatcggcgtggccgccggcatcctgccgtggaacttcccgttcttcctgatcgcgcgcaagctggcgccggcgctgatcaccggcaacaccatcgtgatcaagccgagcgagatcacgccgatcaacgccttcgagttcgcgcgcctggcctcgcagaccgacctgccgcgcggcgtgttcaacctggtgggcggcaccggcgccggcgccggcgcgcagctcacctcgcaccgcgacgtgggcatcgtgtcgttcaccggcagcgtggagaccggcacgcgcatcatgaccgcggcgtcgaagaacctcacgcgcgtgaacctcgagctcggcggcaaggcaccggccatcgtgctggccgacgccgacctcgacctggcggtgaaggccatctacgactcgcgcgtgatcaacaccggacaggtgtgcaactgcgccgagcgcgtgtacgtgcagcgcaaggtggccgacgagttcaccagcaagatcgccgcgcgcatggccggcacgctgtacggcgacccgctggcccagcccgacgtggcgatgggtccgctggtcagccaggccggcctcgacaaggtggcgggcatggtggaccgcgcccgcgcggccggcgccagcatcgtgcaaggtggccgcaaggccaaccgcgacaagggctaccactacgagcccaccgtcatcgcgaactgcagcgccgacatggagatcatgcgcaaggagatcttcgggccggtgctgccgatccaggtggtggacgagctcgacgaggcgatcgcgctggcgaacgactccgactacggcctgacctcgtcgatcttcaccaaggacctgaactcggccatgcgcgcggtgcgcgacctgcagttcggcgagacctacgtgaaccgcgagcacttcgaggcgatgcagggcttccacgccggccgcaagaagtcgggcatcggcggggccgatggcaagcacggcctgtacgagttcaccgagacgcacgtggtctacatccagcacggctga

SEQ ID NO: 20 - protein, 479 aa, Methylibium petroleiphilum
  Methylibium petroleiphilum strain PM1 plasmid RPME01 hydroxyisobutyraldehyde dehydrogenase, lactaldehyde dehydrogenase, MpeA0361
MVTEYRNYIDGEFLANRSGALIDVHNPATHELLARVPDAPNDVVDLAVQAARTAQPGWAKLPAIQRAQHLRAIAARLRENVEELAHTITAEQGKVLGLARVEVNFTADYMDYMAEWARRLEGEVLTSDRVGESIFLMRKPIGVAAGILPWNFPFFLIARKLAPALITGNTIVIKPSEITPINAFEFARLASQTDLPRGVFNLVGGTGAGAGAQLTSHRDVGIVSFTGSVETGTRIMTAASKNLTRVNLELGGKAPAIVLADADLDLAVKAIYDSRVINTGQVCNCAERVYVQRKVADEFTSKIAARMAGTLYGDPLAQPDVAMGPLVSQAGLDKVAGMVDRARAAGASIVQGGRKANRDKGYHYEPTVIANCSADMEIMRKEIFGPVLPIQVVDELDEAIALANDSDYGLTSSIFTKDLNSAMRAVRDLQFGETYVNREHFEAMQGFHAGRKKSGIGGADGKHGLYEFTETHVVYIQHG

SEQ ID NO: 21 - DNA, 1689 bp, Methylibium petroleiphilum
  Methylibium petroleiphilum strain PM1 plasmid RPME01 2-hydroxy-isobutyryl CoA-mutase, methylmalonyl-CoA mutase, MpeB0541, MTBE degradation pathway mdpR
atgacctggcttgagccgcagataaagtcccaactccaatcggagcgcaaggactgggaagcgaacgaagtcggcgccttcttgaagaaggcgcccgagcgcaaggagcagttccacacgatcggggacttcccggtccagcgcacctacaccgctgccgacatcgccgacacgccgctggaggacatcggtcttccggggcgctacccgttcacgcgcgggccctacccgacgatgtaccgcagccgcacctggacgatgcgccagatcgccggcttcggcaccggcgaggacaccaacaagcgcttcaagtatctgatcgcgcagggccagaccggcatctccaccgacttcgacatgcccacgctgatgggctacgactccgaccacccgatgagcgacggcgaggtcggccgcgagggcgtggcgatcgacacgctggccgacatggaggcgctgctggccgacatcgacctcgagaagatctcggtctcgttcacgatcaacccgagcgcctggatcctgctcgcgatgtacgtggcgctcggcgagaagcgcggctacgacctgaacaagctgtcgggcacggtgcaggccgacatcctgaaggagtacatggcgcagaaggagtacatctacccgatcgcgccgtcggtgcgcatcgtgcgcgacatcatcacctacagcgcgaagaacctgaagcgctacaacccgatcaacatctcgggctaccacatcagcgaggccggctcgtcgccgctgcaggaggcggccttcacgctggccaacctgatcacctacgtgaacgaggtgacggagaccggcatgcacgtcgacgagttcgcgccgcgcctcgccttcttcttcgtgtcgcaaggtgacttcttcgaggaggtagcgaagttccgcgccctacgtcgctgctacgcgaagatcatgaaggagcgcttcggcgcgaagaaccccgagtcgatgcggctgcgctttcactgtcagaccgcggcggcgactttgaccaagccgcagtacatggtcaacgtcgtgcgtacgtcgctgcaggcgctgtcggccgtgctcggcggcgcgcagtcgctgcacaccaacggctacgacgaagccttcgcgatcccgaccgaggatgcgatgaagatggcgctgcgcacgcagcagatcattgccgaggagagtggtgtcgccgacgtgatcgacccgctgggtggcagctactacgtcgaggcgctgaccaccgagtacgagaagaagatcttcgagatcctcgaggaagtcgagaagcgcggtggcaccatcaagctgatcgagcagggctggttccagaagcagattgcggacttcgcttacgagaccgcgctgcgcaagcagtccggccagaagccggtgatcggggtgaaccgcttcgtcgagaacgaagaggacgtcaagatcgagatccacccgtacgacaacacgacggccgaacgccagatttcccgcacgcgccgcgttcgcgccgagcgcgacgaggccaaggtgcaagcgatgctcgaccaactggtggctgtcgccaaggacgagtcccagaacctgatgccgctgaccatcgaactggtgaaggccggcgcaacgatgggggacatcgtcgagaagctgaaggggatctggggtacctaccgcgagacgccggtcttctga

SEQ ID NO: 22 - protein, 562 aa, Methylibium petroleiphilum
  Methylibium petroleiphilum strain PM1 plasmid RPME01 2-hydroxy-isobutyryl CoA-mutase, methylmalonyl-CoA mutase, MpeB0541, MTBE degradation pathway MdpR
MTWLEPQIKSQLQSERKDWEANEVGAFLKKAPERKEQFHTIGDFPVQRTYTAADIADTPLEDIGLPGRYPFTRGPYPTMYRSRTWTMRQIAGFGTGEDTNKRFKYLIAQGQTGISTDFDMPTLMGYDSDHPMSDGEVGREGVAIDTLADMEALLADIDLEKISVSFTINPSAWILLAMYVALGEKRGYDLNKLSGTVQADILKEYMAQKEYIYPIAPSVRIVRDIITYSAKNLKRYNPINISGYHISEAGSSPLQEAAFTLANLITYVNEVTETGMHVDEFAPRLAFFFVSQGDFFEEVAKFRALRRCYAKIMKERFGAKNPESMRLRFHCQTAAATLTKPQYMVNVVRTSLQALSAVLGGAQSLHTNGYDEAFAIPTEDAMKMALRTQQIIAEESGVADVIDPLGGSYYVEALTTEYEKKIFEILEEVEKRGGTIKLIEQGWFQKQIADFAYETALRKQSGQKPVIGVNRFVENEEDVKIEIHPYDNTTAERQISRTRRVRAERDEAKVQAMLDQLVAVAKDESQNLMPLTIELVKAGATMGDIVEKLKGIWGTYRETPVF

SEQ ID NO: 23 - DNA, 411 bp, Methylibium petroleiphilum
  Methylibium petroleiphilum strain PM1 plasmid RPME01 2-hydroxy-isobutyryl CoA-mutase C-terminal domain, methylmalonyl-CoA mutase C-terminal domain/subunit (cobalamin-binding), MpeB0538, MTBE degradation pathway mdpO
atggaccaaatcccgatccgcgttcttctcgccaaagtcggcctcgacggccatgacagaggcgtcaaggtggtcgctcgcgcgctgcgcgacgccggcatggacgtcatctactccggccttcatcgcacgcccgaagaagtggtcaacaccgccatccaggaagacgtggacgtgctgggtgtgagcctcctgtccggcgtgcagctcacggtcttccccaagatcttcaagctcctggaagagagaggcgccggcgacttgatcgtgatcgccggtggcgtgatgccggacgaggacgccgcggccatccgcaaactgggcgtgcgcgaggtgctcctgcaggacacgccgccgcaggccatcatcgactcgatccgcgccttggtcgccgcgcgcggcgcccgctga

SEQ ID NO: 24 - protein, 136 aa, Methylibium petroleiphilum
  Methylibium petroleiphilum strain PM1 plasmid RPME01 2-hydroxy-isobutyryl CoA-mutase C-terminal domain, methylmalonyl-CoA mutase C-terminal domain/subunit (cobalamin-binding), MpeB0538, MTBE degradation pathway MdpO
MDQIPIRVLLAKVGLDGHDRGVKVVARALRDAGMDVIYSGLHRTPEEVVNTAIQEDVDVLGVSLLSGVQLTVFPKIFKLLEERGAGDLIVIAGGVMPDEDAAAIRKLGVREVLLQDTPPQAIIDSIRALVAARGAR

SEQ ID NO: 25 - DNA, 858 bp, Methylibium petroleiphilum
  Methylibium petroleiphilum strain PM1 plasmid RPME01 3-hydroxybutyryl-CoA dehydrogenase, 3-hydroxyacyl-CoA dehydrogenase, MpeB0547, MTBE degradation pathway mdpX
atggcaaaccctccaggctccatcggcgtcatcggcgccggcaccatgggcaacggaatcgcgcaggtctgcgcggtggccggcctcaacgtgacgatgttggacgtcgacgacgccgcgttgaagcgcggcatggacaccatcatccgcaatctcgaccgcatggtggcgaaagagaagctgacggccagcgcccgcgatgccgcgctggcgaagatcagtaccggtctggactatggcgcgctgcagtccgccgatatggtgatcgaggctgcgacggagaacctgggactcaagctgaagatcctgcggcaagtcgccaactgcgtcggcaaggacgcgatcattgcgacgaacacctcgtcgatctcgatcacccagctgggcgctgtgctcgacgcgccggagtgcttcattggcatccactttttcaatcccgtgccgctgatgtcgctgctggaggtcatccgcggcgtgcagacgtcggacgcgacccatgctgccacgatggcgtttgcccagaaggtgggcaaggcgccgatcacggtccgcaacagccccggtttcgtggtcaatcgcatcctgtgcccgatgatcaacgaggccatcttcgtcctgcaggaaggcctggcgtctgccgaaggcattgatgtcggcatgcgcctgggatgcaaccatccgatcggtccgctagcactggccgacatgatcggcctcgacaccttgttgtcgatcatgggcgtgctttacgacgagtttaacgatcccaagtaccgcccagcgctgctgctgaaggagatggtcgccgccggccgcctcggccggaagaccaagcaagggttctacagctactcctga

SEQ ID NO: 26 - protein, 285 aa, Methylibium petroleiphilum
  Methylibium petroleiphilum strain PM1 plasmid RPME01 3-hydroxybutyryl-CoA dehydrogenase, 3-hydroxyacyl-CoA dehydrogenase, MpeB0547, MTBE degradation pathway MdpX
MANPPGSIGVIGAGTMGNGIAQVCAVAGLNVTMLDVDDAALKRGMDTIIRNLDRMVAKEKLTASARDAALAKISTGLDYGALQSADMVIEAATENLGLKLKILRQVANCVGKDAIIATNTSSISITQLGAVLDAPECFIGIHFFNPVPLMSLLEVIRGVQTSDATHAATMAFAQKVGKAPITVRNSPGFVVNRILCPMINEAIFVLQEGLASAEGIDVGMRLGCNHPIGPLALADMIGLDTLLSIMGVLYDEFNDPKYRPALLLKEMVAAGRLGRKTKQGFYSYS

SEQ ID NO: 27 - DNA, 1185 bp, Methylibium petroleiphilum
  Methylibium petroleiphilum strain PM1 plasmid RPME01 acetyl-CoA-acetyltransferase, MpeA3367
atgtccaccgatgatccggtcgtgatcgtgtcggctgcgcgaacgccgatcggcgggttgctcggcgacctggcggcgctggcggcctgggaactgggcgccgtcgcgatccgcgccgcggtcgaacgcgccggcgtgccgggcgacgccgtcgacgaggtgctgatgggcaattgcctgatggcgggccagggccaggcgccggcccgccaggcggcgcgcaaggccggccttccggactcggccggcgcggtgacgctgtcgaagatgtgcggctccggcatgcgcgcgctgatgttcggccatgacatgctggcggccggctcggccgaggtggtggtggccggcggcatggagagcatgacgaacgcaccgcacctgagcttcgtgcgcaaggggctgaagtacggcgcggcggtgctgtacgaccacatggcgctcgacggcctggaggacgcctacgagcgcggcaagtcgatgggcgtattcgccgaacagtgcgtcagctattacagcttccggcgcgaggcgatggacgcgttcgcggtggcgtcgacgcagcgcgcgatcgcggcccacaacgacggcagcttcgactgggagatcgcgccggtcacgctggccggcagggcgggcgacgtgaccgtcgaccgcgacgagcagcccttcaaggccaagctcgacaagatcacggcgctgaagccggccttcggcaaggacggcacgatcaccgccgccacctcgtcgagcatctccgacggcgccgcggcgctggtgctgatgcgtgcctccaccgcccgcgcgcgcggcctcgcgccgatcgccgtgctgcgcgcgcacgcggtgcatgcgcaggcgccggcctggttctccaccgcgccggccggcgcgatccgcaaggtgctgcagaagaccggctggtcggtgcgcgacgtcgacctgtgggagatcaacgaggccttcgccgcggtgacgatggcggcgatgaccgatttcgagctgccgcacgagcgtgtcaacgtgcacggcggggcctgcgcgctgggccacccgatcggcgcgtcgggggcccgcatcgtcgtgacgctgctgggcgcgctgcagcggcgcgggctgcggcgtggcgtggcggcgctgtgcatcggcggcggcgaggccacggcactggcggtcgagctgccttga

SEQ ID NO: 28 - protein, 394 aa, Methylibium petroleiphilum
  Methylibium petroleiphilum strain PM1 plasmid RPME01 acetyl-CoA-acetyltransferase, MpeA3367
MSTDDPVVIVSAARTPIGGLLGDLAALAAWELGAVAIRAAVERAGVPGDAVDEVLMGNCLMAGQGQAPARQAARKAGLPDSAGAVTLSKMCGSGMRALMFGHDMLAAGSAEVVVAGGMESMTNAPHLSFVRKGLKYGAAVLYDHMALDGLEDAYERGKSMGVFAEQCVSYYSFRREAMDAFAVASTQRAIAAHNDGSFDWEIAPVTLAGRAGDVTVDRDEQPFKAKLDKITALKPAFGKDGTITAATSSSISDGAAALVLMRASTARARGLAPIAVLRAHAVHAQAPAWFSTAPAGAIRKVLQKTGWSVRDVDLWEINEAFAAVTMAAMTDFELPHERVNVHGGACALGHPIGASGARIVVTLLGALQRRGLRRGVAALCIGGGEATALAVELP

SEQ ID NO: 29 - protein, 477 aa, Methylibium petroleiphilum
  Methylibium petroleiphilum strain PM1 plasmid RPME01 2-hydroxy-isobutyryl-CoA ligase, phenylacetate-CoA ligase, MpeB0539, MTBE degradation pathway MdpP
MEEWKFPVEYDENYLPPADSRYWFPRRETMPAAERDKAILGRLQQVCQYAWDTSPFYRRKWEEANFHPSQLKSLEDFETRVPVIKKTDLRESQAAHPPFGDYVCVPDSEIFHVHGTSGTTGRPTAFGIGRADWRAIANAHARIMWGMGIRPGDLVCVAAVFSLYMGSWGALAGAERLRAKAFPFGAGAPGMSARLVQWLDTMKPAAFYGTPSYAIHLAEVAREEKLNPRDFGLKCLFFSGEPGASVPGVKDRIEEAYGAKVYDCGSMAEMSPFMNVAGTEQSNDGMLCWQDIIYTEVCDPANMRRVPYGQRGTPVYTHLERTSQPMIRLLSGDLTLWTNDENPCGRTYPRLPQGIFGRIDDMFTIRGENIYPSEIDAALNQMSGYGGEHRIVITRESAMDELLLRVEPSESVHAAGAAALETFRAEASHRVQTVLGVRAKVELVAPNSIARTDFKARRVIDDRDVFRALNQQLQSSA

SEQ ID NO: 30 - protein, 33 aa, Artificial Sequence
  sequence unique to methanesulfonate monooxygenase (msmA) of methanesulfonate utilizers lacking in Methylibium petroleiphilum PM1 msmA
CXHXXXXXXXXXXXXXXXXXXXXXXXXXXCXXH

SEQ ID NO: 31 - protein, 7 aa, Artificial Sequence
  mdpE (mpeB0558) dehydrogenase conserved motif
GQHKGSA

SEQ ID NO: 32 - protein, 5 aa, Artificial Sequence
  mpeA2443 esterase conserved motif active site residues
GXSAG

SEQ ID NO: 33 - protein, 23 aa, Artificial Sequence
  MdpJ N-terminal Rieske-type [2Fe-2S] domain
CXHXXXXXXXXXXXXXXXXCXXH

SEQ ID NO: 34 - protein, 5 aa, Artificial Sequence
  flavin mononucleotide (FMN) isoalloxazine-binding domain, MdpK domain conserved in class IA oxygenase ferredoxin reductase
RXYSL

SEQ ID NO: 35 - protein, 7 aa, Artificial Sequence
  NAD ribose-binding domain, MdpK domain conserved in class IA oxygenase ferredoxin reductase
GGIGITP

SEQ ID NO: 36 - protein, 39 aa, Artificial Sequence
  [2Fe-2S] ferredoxin-binding domain, MdpK domain conserved in class IA oxygenase ferredoxin reductase
CXXXXCXXCXXXXXXXXXXXXXXXXXXXXXXXXXXXXXC